SEQUENCE LISTING (358 sequences)

SEQ ID NO: 1 - protein, 15 aa, Homo sapiens
LCPDCPLLAPLNDSR

SEQ ID NO: 2 - protein, 28 aa, Homo sapiens
VVHAVEVALATFNAESNGSYLQLVEISR

SEQ ID NO: 3 - protein, 32 aa, Homo sapiens
RPTGEVYDIEIDTLETTCHVLDPTPLANCSVR

SEQ ID NO: 4 - protein, 22 aa, Homo sapiens
AALAAFNAQNNGSNFQLEEISR

SEQ ID NO: 5 - protein, 22 aa, Homo sapiens
AATCINPLNGSVCERPANHSAK

SEQ ID NO: 6 - protein, 22 aa, Homo sapiens
ADGTVNQIEGEATPVNLTEPAK

SEQ ID NO: 7 - protein, 26 aa, Homo sapiens
ADGTVNQIEGEATPVNLTEPAKLEVK

SEQ ID NO: 8 - protein, 24 aa, Homo sapiens
ADTHDEILEGLNFNLTEIPEAQIH

SEQ ID NO: 9 - protein, 32 aa, Homo sapiens
ADTHDEILEGLNFNLTEIPEAQIHEGFQELLR

SEQ ID NO: 10 - protein, 24 aa, Homo sapiens
AELSNHTRPVILVPGCLGNQLEAK

SEQ ID NO: 11 - protein, 22 aa, Homo sapiens
AFENVTDLQWLILDHNLLENSK

SEQ ID NO: 12 - protein, 24 aa, Homo sapiens
AFHYNVSSHGCQLLPWTQHSPHTR

SEQ ID NO: 13 - protein, 20 aa, Homo sapiens
AFITNFSMIIDGMTYPGIIK

SEQ ID NO: 14 - protein, 22 aa, Homo sapiens
AFITNFSMIIDGMTYPGIIKEK

SEQ ID NO: 15 - protein, 21 aa, Homo sapiens
AGAFLGLTNVAVMNLSGNCLR

SEQ ID NO: 16 - protein, 14 aa, Homo sapiens
AGLQAFFQVQECNK

SEQ ID NO: 17 - protein, 33 aa, Homo sapiens
AHLNVSGIPCSVLLADVEDLIQQQISNDTVSPR

SEQ ID NO: 18 - protein, 16 aa, Homo sapiens
ALPQPQNVTSLLGCTH

SEQ ID NO: 19 - protein, 22 aa, Homo sapiens
ALQAVYSMMSWPDDVPPEGWNR

SEQ ID NO: 20 - protein, 47 aa, Homo sapiens
AMMAFTADLFSLVAQTSTCPNLILSPLSVALALSHLALGAQNHTLQR

SEQ ID NO: 21 - protein, 26 aa, Homo sapiens
ANLSSQALQMSLDYGFVTPLTSMSIR

SEQ ID NO: 22 - protein, 34 aa, Homo sapiens
APDKNVIFSPLSISTALAFLSLGAHNTTLTEILK

SEQ ID NO: 23 - protein, 14 aa, Homo sapiens
AQLLQGLGFNLTER

SEQ ID NO: 24 - protein, 26 aa, Homo sapiens
AQVIINITDVDEPPIFQQPFYHFQLK

SEQ ID NO: 25 - protein, 29 aa, Homo sapiens
AREDIFMETLKDIVEYYNDSNGSHVLQGR

SEQ ID NO: 26 - protein, 30 aa, Homo sapiens
AVLQLNEEGVDTAGSTGVTLNLTSKPIILR

SEQ ID NO: 27 - protein, 18 aa, Homo sapiens
AVNITSENLIDDVVSLIR

SEQ ID NO: 28 - protein, 20 aa, Homo sapiens
AYLLPAPPAPGNASESEEDR

SEQ ID NO: 29 - protein, 17 aa, Homo sapiens
CATPHGDNASLEATFVK

SEQ ID NO: 30 - protein, 13 aa, Homo sapiens
CGLVPVLAENYNK

SEQ ID NO: 31 - protein, 16 aa, Homo sapiens
CGNCSLTTLKDEDFCK

SEQ ID NO: 32 - protein, 17 aa, Homo sapiens
CGNCSLTTLKDEDFCKR

SEQ ID NO: 33 - protein, 13 aa, Homo sapiens
CIQANYSLMENGK

SEQ ID NO: 34 - protein, 15 aa, Homo sapiens
CIQANYSLMENGKIK

SEQ ID NO: 35 - protein, 22 aa, Homo sapiens
CMWSSALNSLNLSFAGLEQVPK

SEQ ID NO: 36 - protein, 22 aa, Homo sapiens
CSDGWSFDATTLDDNGTMLFFK

SEQ ID NO: 37 - protein, 19 aa, Homo sapiens
DFVNASSKYEITTIHNLFR

SEQ ID NO: 38 - protein, 26 aa, Homo sapiens
DHENGTGTNTYAALNSVYLMMNNQMR

SEQ ID NO: 39 - protein, 18 aa, Homo sapiens
DIVEYYNDSNGSHVLQGR

SEQ ID NO: 40 - protein, 27 aa, Homo sapiens
DKICDLLVANNHFAHFFAPQNLTNMNK

SEQ ID NO: 41 - protein, 41 aa, Homo sapiens
DMTEVISSLENANYKDHENGTGTNTYAALNSVYLMMNNQMR

SEQ ID NO: 42 - protein, 18 aa, Homo sapiens
DQCIVDDITYNVNDTFHK

SEQ ID NO: 43 - protein, 19 aa, Homo sapiens
DQCIVDDITYNVNDTFHKR

SEQ ID NO: 44 - protein, 33 aa, Homo sapiens
DRQDGEEVLQCMPVCGRPVTPIAQNQTTLGSSR

SEQ ID NO: 45 - protein, 14 aa, Homo sapiens
DSVSVVLGQHFFNR

SEQ ID NO: 46 - protein, 29 aa, Homo sapiens
DTAVFECLPQHAMFGNDTITCTTHGNWTK

SEQ ID NO: 47 - protein, 34 aa, Homo sapiens
DTAVFECLPQHAMFGNDTITCTTHGNWTKLPECR

SEQ ID NO: 48 - protein, 19 aa, Homo sapiens
DVQIIVFPEDGIHGFNFTR

SEQ ID NO: 49 - protein, 27 aa, Homo sapiens
EDIFMETLKDIVEYYNDSNGSHVLQGR

SEQ ID NO: 50 - protein, 25 aa, Homo sapiens
EEQYNSTYRVVSVLTVLHQDWLNGK

SEQ ID NO: 51 - protein, 28 aa, Homo sapiens
EEQYNSTYRVVSVLTVLHQDWLNGKEYK

SEQ ID NO: 52 - protein, 33 aa, Homo sapiens
EGDHEFLEVPEAQEDVEATFPVHQPGNYSCSYR

SEQ ID NO: 53 - protein, 19 aa, Homo sapiens
EGYSNISYIVVNHQGISSR

SEQ ID NO: 54 - protein, 24 aa, Homo sapiens
EHEAQSNASLDVFLGHTNVEELMK

SEQ ID NO: 55 - protein, 16 aa, Homo sapiens
EHEGAIYPDNTTDFQR

SEQ ID NO: 56 - protein, 20 aa, Homo sapiens
EHETCLAPELYNGNYSTTQK

SEQ ID NO: 57 - protein, 26 aa, Homo sapiens
EHYNLSAATCSPGQMCGHYTQVVWAK

SEQ ID NO: 58 - protein, 17 aa, Homo sapiens
ELDREVYPWYNLTVEAK

SEQ ID NO: 59 - protein, 17 aa, Homo sapiens
ELHHLQEQNVSNAFLDK

SEQ ID NO: 60 - protein, 25 aa, Homo sapiens
ELHHLQEQNVSNAFLDKGEFYIGSK

SEQ ID NO: 61 - protein, 22 aa, Homo sapiens
ELPGVCNETMMALWEECKPCLK

SEQ ID NO: 62 - protein, 20 aa, Homo sapiens
ENLTAPGSDSAVFFEQGTTR

SEQ ID NO: 63 - protein, 15 aa, Homo sapiens
ERSWPAVGNCSSALR

SEQ ID NO: 64 - protein, 28 aa, Homo sapiens
EVNTSGFAPARPPPQPGSTTFWAWSVLR

SEQ ID NO: 65 - protein, 25 aa, Homo sapiens
EVSFLNCSLDNGGCTHYCLEEVGWR

SEQ ID NO: 66 - protein, 13 aa, Homo sapiens
EVYPWYNLTVEAK

SEQ ID NO: 67 - protein, 29 aa, Homo sapiens
EWEKELHHLQEQNVSNAFLDKGEFYIGSK

SEQ ID NO: 68 - protein, 15 aa, Homo sapiens
EYESYSDFERNVTEK

SEQ ID NO: 69 - protein, 15 aa, Homo sapiens
FCRDNYTDLVAIQNK

SEQ ID NO: 70 - protein, 34 aa, Homo sapiens
FDFQGTCEYLLSAPCHGPPLGAENFTVTVANEHR

SEQ ID NO: 71 - protein, 39 aa, Homo sapiens
FEDGVLDPDYPRNISDGFDGIPDNVDAALALPAHSYSGR

SEQ ID NO: 72 - protein, 17 aa, Homo sapiens
FEVDSPVYNATWSASLK

SEQ ID NO: 73 - protein, 38 aa, Homo sapiens
FGHSAVLHNSTMYVFGGFNSLLLSDILVFTSEQCDAHR

SEQ ID NO: 74 - protein, 22 aa, Homo sapiens
FHDVSESTHWTPFLNASVHYIR

SEQ ID NO: 75 - protein, 12 aa, Homo sapiens
FLNNGTCTAEGK

SEQ ID NO: 76 - protein, 19 aa, Homo sapiens
FLTEVEKNATALYHVEAFK

SEQ ID NO: 77 - protein, 34 aa, Homo sapiens
FNFQGTCEYLLSAPCHGPPLGAENFTVTVANEHR

SEQ ID NO: 78 - protein, 20 aa, Homo sapiens
FNLTETSEAEIHQSFQHLLR

SEQ ID NO: 79 - protein, 16 aa, Homo sapiens
FNPGAESVVLSNSTLK

SEQ ID NO: 80 - protein, 15 aa, Homo sapiens
FNSSYLQGTNQITGR

SEQ ID NO: 81 - protein, 43 aa, Homo sapiens
FQSPAGTEALFELHNISVADSANYSCVYVDLKPPFGGSAPSER

SEQ ID NO: 82 - protein, 16 aa, Homo sapiens
FSDGLESNSSTQFEVK

SEQ ID NO: 83 - protein, 17 aa, Homo sapiens
FSDGLESNSSTQFEVKK

SEQ ID NO: 84 - protein, 30 aa, Homo sapiens
FSLLGHASISCTVENETIGVWRPSPPTCEK

SEQ ID NO: 85 - protein, 18 aa, Homo sapiens
FSYSKNETYQLFLSYSSK

SEQ ID NO: 86 - protein, 15 aa, Homo sapiens
FVGTPEVNQTTLYQR

SEQ ID NO: 87 - protein, 35 aa, Homo sapiens
FVQAICEGDDCQPPAYTYNNITCASPPEVVGLDLR

SEQ ID NO: 88 - protein, 19 aa, Homo sapiens
FVQGNSTEVACHPGYGLPK

SEQ ID NO: 89 - protein, 14 aa, Homo sapiens
GAFISNFSMTVDGK

SEQ ID NO: 90 - protein, 16 aa, Homo sapiens
GCNDSDVLAVAGFALR

SEQ ID NO: 91 - protein, 26 aa, Homo sapiens
GCSCFSDWQGPGCSVPVPANQSFWTR

SEQ ID NO: 92 - protein, 22 aa, Homo sapiens
GCVLLSYLNETVTVSASLESVR

SEQ ID NO: 93 - protein, 39 aa, Homo sapiens
GDSGGPLVCMDANNVTYVWGVVSWGENCGKPEFPGVYTK

SEQ ID NO: 94 - protein, 22 aa, Homo sapiens
GETHEQVHSILHFKDFVNASSK

SEQ ID NO: 95 - protein, 33 aa, Homo sapiens
GETHEQVHSILHFKDFVNASSKYEITTIHNLFR

SEQ ID NO: 96 - protein, 21 aa, Homo sapiens
GFGVAIVGNYTAALPTEAALR

SEQ ID NO: 97 - protein, 22 aa, Homo sapiens
GFLALYQTVAVNYSQPISEASR

SEQ ID NO: 98 - protein, 31 aa, Homo sapiens
GGETAQSADPQWEQLNNKNLSMPLLPADFHK

SEQ ID NO: 99 - protein, 28 aa, Homo sapiens
GGNSNGALCHFPFLYNNHNYTDCTSEGR

SEQ ID NO: 100 - protein, 29 aa, Homo sapiens
GGNSNGALCHFPFLYNNHNYTDCTSEGRR

SEQ ID NO: 101 - protein, 26 aa, Homo sapiens
GHFIYKNVSEDLPLPTFSPTLLGDSR

SEQ ID NO: 102 - protein, 23 aa, Homo sapiens
GLKFNLTETSEAEIHQSFQHLLR

SEQ ID NO: 103 - protein, 12 aa, Homo sapiens
GLNLTEDTYKPR

SEQ ID NO: 104 - protein, 21 aa, Homo sapiens
GLTFQQNASSMCGPDQDTAIR

SEQ ID NO: 105 - protein, 21 aa, Homo sapiens
GLTFQQNASSMCVPDQDTAIR

SEQ ID NO: 106 - protein, 26 aa, Homo sapiens
GMNLTVFGGTVTAFLGIPYAQPPLGR

SEQ ID NO: 107 - protein, 35 aa, Homo sapiens
GNEANYYSNATTDEHGLVQFSINTTNVMGTSLTVR

SEQ ID NO: 108 - protein, 25 aa, Homo sapiens
GNVAVTVSGHTCQHWSAQTPHTHNR

SEQ ID NO: 109 - protein, 22 aa, Homo sapiens
GPSTPLPEDPNWNVTEFHTTPK

SEQ ID NO: 110 - protein, 14 aa, Homo sapiens
GTANTTTAGVPCQR

SEQ ID NO: 111 - protein, 24 aa, Homo sapiens
GTGNDTVLNVALLNVISNQECNIK

SEQ ID NO: 112 - protein, 25 aa, Homo sapiens
GVTSVSQIFHSPDLAIRDTFVNASR

SEQ ID NO: 113 - protein, 10 aa, Homo sapiens
HANWTLTPLK

SEQ ID NO: 114 - protein, 16 aa, Homo sapiens
HEEGHMLNCTCFGQGR

SEQ ID NO: 115 - protein, 22 aa, Homo sapiens
HGIQYFNNNTQHSSLFMLNEVK

SEQ ID NO: 116 - protein, 23 aa, Homo sapiens
HGIQYFNNNTQHSSLFMLNEVKR

SEQ ID NO: 117 - protein, 22 aa, Homo sapiens
HGIQYFNNNTQHSSLFTLNEVK

SEQ ID NO: 118 - protein, 23 aa, Homo sapiens
HGIQYFNNNTQHSSLFTLNEVKR

SEQ ID NO: 119 - protein, 21 aa, Homo sapiens
HGVIISSTVDTYENGSSVEYR

SEQ ID NO: 120 - protein, 38 aa, Homo sapiens
HLQMDIHIFEPQGISFLETESTFMTNQLVDALTTWQNK

SEQ ID NO: 121 - protein, 22 aa, Homo sapiens
HYLVSNISHDTVLQCHFTCSGK

SEQ ID NO: 122 - protein, 14 aa, Homo sapiens
HYTNSSQDVTVPCR

SEQ ID NO: 123 - protein, 43 aa, Homo sapiens
HYYIAAEEIIWNYAPSGIDIFTKENLTAPGSDSAVFFEQGTTR

SEQ ID NO: 124 - protein, 31 aa, Homo sapiens
IADAHLDRVENTTVYYLVLDVQESDCSVLSR

SEQ ID NO: 125 - protein, 25 aa, Homo sapiens
ICDLLVANNHFAHFFAPQNLTNMNK

SEQ ID NO: 126 - protein, 12 aa, Homo sapiens
IDSTGNVTNELR

SEQ ID NO: 127 - protein, 33 aa, Homo sapiens
IITILEEEMNVSVCGLYTYGKPVPGHVTVSICR

SEQ ID NO: 128 - protein, 18 aa, Homo sapiens
INNDFNYEFYNSTWSYVK

SEQ ID NO: 129 - protein, 18 aa, Homo sapiens
IPCSQPPQIEHGTINSSR

SEQ ID NO: 130 - protein, 13 aa, Homo sapiens
ISEENETTCYMGK

SEQ ID NO: 131 - protein, 17 aa, Homo sapiens
ISNSSDTVECECSENWK

SEQ ID NO: 132 - protein, 35 aa, Homo sapiens
ISNSSDTVECECSENWKGEACDIPHCTDNCGFPHR

SEQ ID NO: 133 - protein, 44 aa, Homo sapiens
ITPNLAEFAFSLYRQLAHQSNSTNIFFSPVSIATAFAMLSLGTK

SEQ ID NO: 134 - protein, 12 aa, Homo sapiens
ITYSIVQTNCSK

SEQ ID NO: 135 - protein, 23 aa, Homo sapiens
ITYSIVQTNCSKENFLFLTPDCK

SEQ ID NO: 136 - protein, 21 aa, Homo sapiens
IVGGTNSSWGEWPWQVSLQVK

SEQ ID NO: 137 - protein, 30 aa, Homo sapiens
IVLDPSGSMNIYLVLDGSDSIGASNFTGAK

SEQ ID NO: 138 - protein, 18 aa, Homo sapiens
IYPGVDFGGEELNVTFVK

SEQ ID NO: 139 - protein, 13 aa, Homo sapiens
IYSGILNLSDITK

SEQ ID NO: 140 - protein, 21 aa, Homo sapiens
IYSNHSALESLALIPLQAPLK

SEQ ID NO: 141 - protein, 21 aa, Homo sapiens
KAFITNFSMIIDGMTYPGIIK

SEQ ID NO: 142 - protein, 23 aa, Homo sapiens
KAFITNFSMIIDGMTYPGIIKEK

SEQ ID NO: 143 - protein, 17 aa, Homo sapiens
KCGNCSLTTLKDEDFCK

SEQ ID NO: 144 - protein, 18 aa, Homo sapiens
KDFEDLYTPVNGSIVIVR

SEQ ID NO: 145 - protein, 21 aa, Homo sapiens
KEHETCLAPELYNGNYSTTQK

SEQ ID NO: 146 - protein, 31 aa, Homo sapiens
KIVLDPSGSMNIYLVLDGSDSIGASNFTGAK

SEQ ID NO: 147 - protein, 18 aa, Homo sapiens
KLHINHNNLTESVGPLPK

SEQ ID NO: 148 - protein, 12 aa, Homo sapiens
KLINDYVKNGTR

SEQ ID NO: 149 - protein, 14 aa, Homo sapiens
KLPPGLLANFTLLR

SEQ ID NO: 150 - protein, 19 aa, Homo sapiens
KNQSVNVFLGHTAIDEMLK

SEQ ID NO: 151 - protein, 16 aa, Homo sapiens
KQVHFFVNASDVDNVK

SEQ ID NO: 152 - protein, 16 aa, Homo sapiens
KVCQDCPLLAPLNDTR

SEQ ID NO: 153 - protein, 22 aa, Homo sapiens
LAGKPTHVNVSVVMAEVDGTCY

SEQ ID NO: 154 - protein, 14 aa, Homo sapiens
LANLTQGEDQYYLR

SEQ ID NO: 155 - protein, 25 aa, Homo sapiens
LATALSLSNKFVEGSHNSTVSLTTK

SEQ ID NO: 156 - protein, 20 aa, Homo sapiens
LDAPTNLQFVNETDSTVLVR

SEQ ID NO: 157 - protein, 18 aa, Homo sapiens
LDPVSLQTLQTWNTSYPK

SEQ ID NO: 158 - protein, 18 aa, Homo sapiens
LDRENISEYHLTAVIVDK

SEQ ID NO: 159 - protein, 33 aa, Homo sapiens
LDRENISEYHLTAVIVDKDTGENLETPSSFTIK

SEQ ID NO: 160 - protein, 27 aa, Homo sapiens
LEDLEVTGSSFLNLSTNIFSNLTSLGK

SEQ ID NO: 161 - protein, 25 aa, Homo sapiens
LEPVHLQLQCMSQEQLAQVAANATK

SEQ ID NO: 162 - protein, 19 aa, Homo sapiens
LETTVNYTDSQRPICLPSK

SEQ ID NO: 163 - protein, 24 aa, Homo sapiens
LFGDKSLTFNETYQDISELVYGAK

SEQ ID NO: 164 - protein, 16 aa, Homo sapiens
LGACNDTLQQLMEVFK

SEQ ID NO: 165 - protein, 23 aa, Homo sapiens
LGACNDTLQQLMEVFKFDTISEK

SEQ ID NO: 166 - protein, 34 aa, Homo sapiens
LGACNDTLQQLMEVFKFDTISEKTSDQIHFFFAK

SEQ ID NO: 167 - protein, 25 aa, Homo sapiens
LGHCPDPVLVNGEFSSSGPVNVSDK

SEQ ID NO: 168 - protein, 19 aa, Homo sapiens
LGSFEGLVNLTFIHLQHNR

SEQ ID NO: 169 - protein, 30 aa, Homo sapiens
LGSLQELFLDSNNISELPPQVFSQLFCLER

SEQ ID NO: 170 - protein, 21 aa, Homo sapiens
LGSYPVGGNVSFECEDGFILR

SEQ ID NO: 171 - protein, 17 aa, Homo sapiens
LGTSLSSGHVLMNGTLK

SEQ ID NO: 172 - protein, 17 aa, Homo sapiens
LHINHNNLTESVGPLPK

SEQ ID NO: 173 - protein, 24 aa, Homo sapiens
LKELPGVCNETMMALWEECKPCLK

SEQ ID NO: 174 - protein, 33 aa, Homo sapiens
LLLSQLDSHPSHSAVVNWTSYASSIEALSSGNK

SEQ ID NO: 175 - protein, 12 aa, Homo sapiens
LNAENNATFYFK

SEQ ID NO: 176 - protein, 40 aa, Homo sapiens
LNDTLDYECHDGYESNTGSTTGSIVCGYNGWSDLPICYER

SEQ ID NO: 177 - protein, 11 aa, Homo sapiens
LNVEAANWTVR

SEQ ID NO: 178 - protein, 30 aa, Homo sapiens
LPASLAEYTVTQLRPNATYSVCVMPLGPGR

SEQ ID NO: 179 - protein, 13 aa, Homo sapiens
LPPGLLANFTLLR

SEQ ID NO: 180 - protein, 25 aa, Homo sapiens
LPTQNITFQTESSVAEQEAEFQSPK

SEQ ID NO: 181 - protein, 16 aa, Homo sapiens
LPYQGNATMLVVLMEK

SEQ ID NO: 182 - protein, 17 aa, Homo sapiens
LQAILGVPWKDKNCTSR

SEQ ID NO: 183 - protein, 19 aa, Homo sapiens
LQAPLNYTEFQKPICLPSK

SEQ ID NO: 184 - protein, 13 aa, Homo sapiens
LQNNENNISCVER

SEQ ID NO: 185 - protein, 17 aa, Homo sapiens
LSDLSINSTECLHVHCR

SEQ ID NO: 186 - protein, 30 aa, Homo sapiens
LSHNELADSGIPGNSFNVSSLVELDLSYNK

SEQ ID NO: 187 - protein, 27 aa, Homo sapiens
LSLHRPALEDLLLGSEANLTCTLTGLR

SEQ ID NO: 188 - protein, 25 aa, Homo sapiens
LSSWVLLMKYLGNATAIFFLPDEGK

SEQ ID NO: 189 - protein, 36 aa, Homo sapiens
LSVDKDQYVEPENVTIQCDSGYGVVGPQSITCSGNR

SEQ ID NO: 190 - protein, 20 aa, Homo sapiens
LTDTICGVGNMSANASDQER

SEQ ID NO: 191 - protein, 30 aa, Homo sapiens
LVSANRLFGDKSLTFNETYQDISELVYGAK

SEQ ID NO: 192 - protein, 30 aa, Homo sapiens
LYHFLLGAWSLNATELDPCPLSPELLGLTK

SEQ ID NO: 193 - protein, 21 aa, Homo sapiens
LYLGSNNLTALHPALFQNLSK

SEQ ID NO: 194 - protein, 22 aa, Homo sapiens
MAGKPTHINVSVVMAEADGTCY

SEQ ID NO: 195 - protein, 22 aa, Homo sapiens
MAGKPTHVNVSVVMAEVDGTCY

SEQ ID NO: 196 - protein, 20 aa, Homo sapiens
MAWPEDHVFISTPSFNYTGR

SEQ ID NO: 197 - protein, 13 aa, Homo sapiens
MDGASNVTCINSR

SEQ ID NO: 198 - protein, 21 aa, Homo sapiens
MLLTFHTDFSNEENGTIMFYK

SEQ ID NO: 199 - protein, 20 aa, Homo sapiens
MLNNNTGIYTCSAQGVWMNK

SEQ ID NO: 200 - protein, 20 aa, Homo sapiens
MLNTSSLLEQLNEQFNWVSR

SEQ ID NO: 201 - protein, 26 aa, Homo sapiens
MPSQAPTGNFYPQPLLNSSMCLEDSR

SEQ ID NO: 202 - protein, 33 aa, Homo sapiens
MQCLAAALKDETNMSGGGEQADILPANYVVKDR

SEQ ID NO: 203 - protein, 29 aa, Homo sapiens
MSNITFLNFDPPIEEFHQYYQHIVTTLVK

SEQ ID NO: 204 - protein, 24 aa, Homo sapiens
MVSHHNLTTGATLINEQWLLTTAK

SEQ ID NO: 205 - protein, 37 aa, Homo sapiens
MVSHHNLTTGATLINEQWLLTTAKNLFLNHSENATAK

SEQ ID NO: 206 - protein, 36 aa, Homo sapiens
MVTAFTTCCTLSEEFACVDNLADLVFGELCGVNENR

SEQ ID NO: 207 - protein, 13 aa, Homo sapiens
NAHGEEKENLTAR

SEQ ID NO: 208 - protein, 32 aa, Homo sapiens
NCGVNCSGDVFTALIGEIASPNYPKPYPENSR

SEQ ID NO: 209 - protein, 29 aa, Homo sapiens
NCQDIDECVTGIHNCSINETCFNIQGGFR

SEQ ID NO: 210 - protein, 30 aa, Homo sapiens
NEEYNKSVQEIQATFFYFTPNKTEDTIFLR

SEQ ID NO: 211 - protein, 18 aa, Homo sapiens
NEMLEIQVFNYSKVFSNK

SEQ ID NO: 212 - protein, 17 aa, Homo sapiens
NGTGHGNSTHHGPEYMR

SEQ ID NO: 213 - protein, 19 aa, Homo sapiens
NHPNITFFVYVSNFTWPIK

SEQ ID NO: 214 - protein, 27 aa, Homo sapiens
NISDGFDGIPDNVDAALALPAHSYSGR

SEQ ID NO: 215 - protein, 34 aa, Homo sapiens
NLASRPYTFHSHGITYYKEHEGAIYPDNTTDFQR

SEQ ID NO: 216 - protein, 13 aa, Homo sapiens
NLFLNHSENATAK

SEQ ID NO: 217 - protein, 25 aa, Homo sapiens
NLFLNHSENATAKDIAPTLTLYVGK

SEQ ID NO: 218 - protein, 26 aa, Homo sapiens
NLFLNHSENATAKDIAPTLTLYVGKK

SEQ ID NO: 219 - protein, 23 aa, Homo sapiens
NNATVHEQVGGPSLTSDLQAQSK

SEQ ID NO: 220 - protein, 40 aa, Homo sapiens
NNMSFVVLVPTHFEWNVSQVLANLSWDTLHPPLVWERPTK

SEQ ID NO: 221 - protein, 28 aa, Homo sapiens
NPPMGGNVVIFDTVITNQEEPYQNHSGR

SEQ ID NO: 222 - protein, 21 aa, Homo sapiens
NPVGLIGAENATGETDPSHSK

SEQ ID NO: 223 - protein, 42 aa, Homo sapiens
NQALNLSLAYSFVTPLTSMVVTKPDDQEQSQVAEKPMEGESR

SEQ ID NO: 224 - protein, 32 aa, Homo sapiens
NSVLNSSTAEHSSPYSEDPIEDPLQPDVTGIR

SEQ ID NO: 225 - protein, 30 aa, Homo sapiens
NVIFSPLSISTALAFLSLGAHNTTLTEILK

SEQ ID NO: 226 - protein, 15 aa, Homo sapiens
QDQCIYNTTYLNVQR

SEQ ID NO: 227 - protein, 22 aa, Homo sapiens
QDQCIYNTTYLNVQRENGTISR

SEQ ID NO: 228 - protein, 27 aa, Homo sapiens
QEDLSVGSVLLTVNATDPDSLQHQTIR

SEQ ID NO: 229 - protein, 15 aa, Homo sapiens
QGGVNATQVLIQHLR

SEQ ID NO: 230 - protein, 16 aa, Homo sapiens
QINSSISGNLWDKDQR

SEQ ID NO: 231 - protein, 30 aa, Homo sapiens
QLAHQSNSTNIFFSPVSIATAFAMLSLGTK

SEQ ID NO: 232 - protein, 31 aa, Homo sapiens
QLDMLDLSNNSLASVPEGLWASLGQPNWDMR

SEQ ID NO: 233 - protein, 20 aa, Homo sapiens
QLEEFLNQSSPFYFWMNGDR

SEQ ID NO: 234 - protein, 29 aa, Homo sapiens
QLEEFLNQSSPFYFWMNGDRIDSLLENDR

SEQ ID NO: 235 - protein, 23 aa, Homo sapiens
QLVEIEKVVLHPNYSQVDIGLIK

SEQ ID NO: 236 - protein, 39 aa, Homo sapiens
QNESHNFSGDIALLELQHSIPLGPNVLPVCLPDNETLYR

SEQ ID NO: 237 - protein, 15 aa, Homo sapiens
QNQCFYNSSYLNVQR

SEQ ID NO: 238 - protein, 18 aa, Homo sapiens
QPQAGLSQANFTLGPVSR

SEQ ID NO: 239 - protein, 21 aa, Homo sapiens
QQQHLFGSNVTDCSGNFCLFR

SEQ ID NO: 240 - protein, 15 aa, Homo sapiens
QVHFFVNASDVDNVK

SEQ ID NO: 241 - protein, 19 aa, Homo sapiens
QVLFLDTVYGNCSTHFTVK

SEQ ID NO: 242 - protein, 27 aa, Homo sapiens
QVQVLQNLTTTYEIVLWQPVTADLIVK

SEQ ID NO: 243 - protein, 34 aa, Homo sapiens
REGDHEFLEVPEAQEDVEATFPVHQPGNYSCSYR

SEQ ID NO: 244 - protein, 17 aa, Homo sapiens
RHEEGHMLNCTCFGQGR

SEQ ID NO: 245 - protein, 29 aa, Homo sapiens
RNPPMGGNVVIFDTVITNQEEPYQNHSGR

SEQ ID NO: 246 - protein, 46 aa, Homo sapiens
SDHGSSISCQPPAEIPGYLPADTVHLAVEFFNLTHLPANLLQGASK

SEQ ID NO: 247 - protein, 21 aa, Homo sapiens
SHAASDAPENLTLLAETADAR

SEQ ID NO: 248 - protein, 21 aa, Homo sapiens
SHEIWTHSCPQSPGNGTDASH

SEQ ID NO: 249 - protein, 26 aa, Homo sapiens
SIPACVPWSPYLFQPNDTCIVSGWGR

SEQ ID NO: 250 - protein, 25 aa, Homo sapiens
SKPTVSSSMEFKYDFNSSMLYSTAK

SEQ ID NO: 251 - protein, 25 aa, Homo sapiens
SKWNITMESYVVHTNYDEYAIFLTK

SEQ ID NO: 252 - protein, 32 aa, Homo sapiens
SLGNVNFTVSAEALESQELCGTEVPSVPEHGR

SEQ ID NO: 253 - protein, 33 aa, Homo sapiens
SLGNVNFTVSAEALESQELCGTEVPSVPEHGRK

SEQ ID NO: 254 - protein, 19 aa, Homo sapiens
SLTFNETYQDISELVYGAK

SEQ ID NO: 255 - protein, 25 aa, Homo sapiens
SPYEMFGDEEVMCLNGNWTEPPQCK

SEQ ID NO: 256 - protein, 21 aa, Homo sapiens
SPYYNVSDEISFHCYDGYTLR

SEQ ID NO: 257 - protein, 21 aa, Homo sapiens
SQILEGLGFNLTELSESDVHR

SEQ ID NO: 258 - protein, 25 aa, Homo sapiens
SRVYLQGLIDCYLFGNSSTVLEDSK

SEQ ID NO: 259 - protein, 25 aa, Homo sapiens
SRYPHKPEINSTTHPGADLQENFCR

SEQ ID NO: 260 - protein, 36 aa, Homo sapiens
SSVITLNTNAELFNQSDIVAHLLSSSSSVIDALQYK

SEQ ID NO: 261 - protein, 22 aa, Homo sapiens
STGKPTLYNVSLVMSDTAGTCY

SEQ ID NO: 262 - protein, 24 aa, Homo sapiens
SVQEIQATFFYFTPNKTEDTIFLR

SEQ ID NO: 263 - protein, 19 aa, Homo sapiens
SVVAPATDGGLNLTSTFLR

SEQ ID NO: 264 - protein, 31 aa, Homo sapiens
TEGRPDMKTELFSSSCPGGIMLNETGQGYQR

SEQ ID NO: 265 - protein, 23 aa, Homo sapiens
TELFSSSCPGGIMLNETGQGYQR

SEQ ID NO: 266 - protein, 33 aa, Homo sapiens
TEVSSNHVLIYLDKVSNQTLSLFFTVLQDVPVR

SEQ ID NO: 267 - protein, 41 aa, Homo sapiens
TEVSSNHVLIYLDKVSNQTLSLFFTVLQDVPVRDLKPAIVK

SEQ ID NO: 268 - protein, 32 aa, Homo sapiens
THTNISESHPNATFSAVGEASICEDDWNSGER

SEQ ID NO: 269 - protein, 13 aa, Homo sapiens
TKPREEQYNSTYR

SEQ ID NO: 270 - protein, 18 aa, Homo sapiens
TLFCNASKEWDNTTTECR

SEQ ID NO: 271 - protein, 20 aa, Homo sapiens
TLNQSSDELQLSMGNAMFVK

SEQ ID NO: 272 - protein, 19 aa, Homo sapiens
TLYETEVFSTDFSNISAAK

SEQ ID NO: 273 - protein, 34 aa, Homo sapiens
TTTVQVPMMHQMEQYYHLVDMELNCTVLQMDYSK

SEQ ID NO: 274 - protein, 15 aa, Homo sapiens
TVIRPFYLTNSSGVD

SEQ ID NO: 275 - protein, 21 aa, Homo sapiens
TVLTPATNHMGNVTFTIPANR

SEQ ID NO: 276 - protein, 24 aa, Homo sapiens
TVLTPATNHMGNVTFTIPANREFK

SEQ ID NO: 277 - protein, 19 aa, Homo sapiens
TVVTYHIPQNSSLENVDSR

SEQ ID NO: 278 - protein, 22 aa, Homo sapiens
TYNVLDMKNTTCQDLQIEVTVK

SEQ ID NO: 279 - protein, 19 aa, Homo sapiens
VASVININPNTTHSTGSCR

SEQ ID NO: 280 - protein, 15 aa, Homo sapiens
VCQDCPLLAPLNDTR

SEQ ID NO: 281 - protein, 21 aa, Homo sapiens
VCQDCPLLAPLNDTRVVHAAK

SEQ ID NO: 282 - protein, 18 aa, Homo sapiens
VDKDLQSLEDILHQVENK

SEQ ID NO: 283 - protein, 33 aa, Homo sapiens
VEGSSSHLVTFTVLPLEIGLHNINFSLETWFGK

SEQ ID NO: 284 - protein, 23 aa, Homo sapiens
VENTTVYYLVLDVQESDCSVLSR

SEQ ID NO: 285 - protein, 15 aa, Homo sapiens
VFHIHNESWVLLTPK

SEQ ID NO: 286 - protein, 44 aa, Homo sapiens
VFPLSLDSTPQDGNVVVACLVQGFFPQEPLSVTWSESGQNVTAR

SEQ ID NO: 287 - protein, 21 aa, Homo sapiens
VGQLQLSHNLSLVILVPQNLK

SEQ ID NO: 288 - protein, 19 aa, Homo sapiens
VIDFNCTTSSVSSALANTK

SEQ ID NO: 289 - protein, 33 aa, Homo sapiens
VIDFNCTTSSVSSALANTKDSPVLIDFFEDTER

SEQ ID NO: 290 - protein, 19 aa, Homo sapiens
VLSNNSDANLELINTWVAK

SEQ ID NO: 291 - protein, 27 aa, Homo sapiens
VLTLNLDQVDFQHAGNYSCVASNVQGK

SEQ ID NO: 292 - protein, 16 aa, Homo sapiens
VLYLAAYNCTLRPVSK

SEQ ID NO: 293 - protein, 14 aa, Homo sapiens
VPGNVTAVLGETLK

SEQ ID NO: 294 - protein, 40 aa, Homo sapiens
VPMMLQSSTISYLHDSELPCQLVQMNYVGNGTVFFILPDK

SEQ ID NO: 295 - protein, 23 aa, Homo sapiens
VSAITLVSATSTTANMTVGPEGK

SEQ ID NO: 296 - protein, 25 aa, Homo sapiens
VSEHIPVYQQEENQTDVWTLLNGSK

SEQ ID NO: 297 - protein, 33 aa, Homo sapiens
VSEHIPVYQQEENQTDVWTLLNGSKDDFLIYDR

SEQ ID NO: 298 - protein, 13 aa, Homo sapiens
VSLTNVSISDEGR

SEQ ID NO: 299 - protein, 19 aa, Homo sapiens
VSNQTLSLFFTVLQDVPVR

SEQ ID NO: 300 - protein, 12 aa, Homo sapiens
VSNVSCQASVSR

SEQ ID NO: 301 - protein, 24 aa, Homo sapiens
VSTVYANNGSVLQGTSVASVYHGK

SEQ ID NO: 302 - protein, 15 aa, Homo sapiens
VTACHSSQPNATLYK

SEQ ID NO: 303 - protein, 30 aa, Homo sapiens
VTISGVYDLGDVLEEMGIADLFTNQANFSR

SEQ ID NO: 304 - protein, 22 aa, Homo sapiens
VTQNLTLIEESLTSEFIHDIDR

SEQ ID NO: 305 - protein, 22 aa, Homo sapiens
VTQVYAENGTVLQGSTVASVYK

SEQ ID NO: 306 - protein, 24 aa, Homo sapiens
VTQVYAENGTVLQGSTVASVYKGK

SEQ ID NO: 307 - protein, 23 aa, Homo sapiens
VTWKPQGAPVEWEEETVTNHTLR

SEQ ID NO: 308 - protein, 16 aa, Homo sapiens
VVLHPNYSQVDIGLIK

SEQ ID NO: 309 - protein, 18 aa, Homo sapiens
VVLHPNYSQVDIGLIKLK

SEQ ID NO: 310 - protein, 21 aa, Homo sapiens
VYIHPFHLVIHNESTCEQLAK

SEQ ID NO: 311 - protein, 13 aa, Homo sapiens
VYKPSAGNNSLYR

SEQ ID NO: 312 - protein, 23 aa, Homo sapiens
VYLQGLIDCYLFGNSSTVLEDSK

SEQ ID NO: 313 - protein, 30 aa, Homo sapiens
VYSGILNQSEIKEDTSFFGVQEIIIHDQYK

SEQ ID NO: 314 - protein, 34 aa, Homo sapiens
WDPEVNCSMAQIQLCPPPPQIPNSHNMTTTLNYR

SEQ ID NO: 315 - protein, 14 aa, Homo sapiens
WFSAGLASNSSWLR

SEQ ID NO: 316 - protein, 15 aa, Homo sapiens
WFYIASAFRNEEYNK

SEQ ID NO: 317 - protein, 23 aa, Homo sapiens
WNITMESYVVHTNYDEYAIFLTK

SEQ ID NO: 318 - protein, 26 aa, Homo sapiens
WNVNAPPTFHSEMMYDNFTLVPVWGK

SEQ ID NO: 319 - protein, 26 aa, Homo sapiens
WVLTAAHCLLYPPWDKNFTENDLLVR

SEQ ID NO: 320 - protein, 12 aa, Homo sapiens
YAEDKFNETTEK

SEQ ID NO: 321 - protein, 29 aa, Homo sapiens
YFYNGTSMACETFQYGGCMGNGNNFVTEK

SEQ ID NO: 322 - protein, 18 aa, Homo sapiens
YGNPNETQNNSTSWPVFK

SEQ ID NO: 323 - protein, 14 aa, Homo sapiens
YKGLNLTEDTYKPR

SEQ ID NO: 324 - protein, 16 aa, Homo sapiens
YLGNATAIFFLPDEGK

SEQ ID NO: 325 - protein, 31 aa, Homo sapiens
YLGNATAIFFLPDEGKLQHLENELTHDIITK

SEQ ID NO: 326 - protein, 30 aa, Homo sapiens
YLHTAVIVSGTMLVFGGNTHNDTSMSHGAK

SEQ ID NO: 327 - protein, 15 aa, Homo sapiens
YNSQNQSNNQFVLYR

SEQ ID NO: 328 - protein, 18 aa, Homo sapiens
YNWSFIHCPACQCNGHSK

SEQ ID NO: 329 - protein, 23 aa, Homo sapiens
YPHKPEINSTTHPGADLQENFCR

SEQ ID NO: 330 - protein, 25 aa, Homo sapiens
YPPTVSMVEGQGEKNVTFWGRPLPR

SEQ ID NO: 331 - protein, 18 aa, Homo sapiens
YQFNTNVVFSNNGTLVDR

SEQ ID NO: 332 - protein, 37 aa, Homo sapiens
YTCEEPYYYMENGGGGEYHCAGNGSWVNEVLGPELPK

SEQ ID NO: 333 - protein, 16 aa, Homo sapiens
YTGNASALFILPDQDK

SEQ ID NO: 334 - protein, 30 aa, Homo sapiens
YTGNASALFILPDQDKMEEVEAMLLPETLK

SEQ ID NO: 335 - protein, 31 aa, Homo sapiens
YTGNASALFILPDQDKMEEVEAMLLPETLKR

SEQ ID NO: 336 - protein, 14 aa, Homo sapiens
AAINKWVSNKTEGR

SEQ ID NO: 337 - protein, 22 aa, Homo sapiens
ALYAWNNGHQILYNVTLFHVIR

SEQ ID NO: 338 - protein, 27 aa, Homo sapiens
EQFCPPPPQIPNAQNMTTTVNYQDGEK

SEQ ID NO: 339 - protein, 15 aa, Homo sapiens
GELNTSIFSSRPIDK

SEQ ID NO: 340 - protein, 39 aa, Homo sapiens
GFYPSDIAVEWESSGQPENNYNTTPPMLDSDGSFFLYSK

SEQ ID NO: 341 - protein, 32 aa, Homo sapiens
GSFPWQAKMVSHHNLTTGATLINEQWLLTTAK

SEQ ID NO: 342 - protein, 14 aa, Homo sapiens
HSHNNNSSDLHPHK

SEQ ID NO: 343 - protein, 9 aa, Homo sapiens
KEDALNETR

SEQ ID NO: 344 - protein, 26 aa, Homo sapiens
KLSSWVLLMKYLGNATAIFFLPDEGK

SEQ ID NO: 345 - protein, 31 aa, Homo sapiens
NMASRPYSIYPHGVTFSPYEDEVNSSFTSGR

SEQ ID NO: 346 - protein, 16 aa, Homo sapiens
SVTLQIYNHSLTLSAR

SEQ ID NO: 347 - protein, 13 aa, Homo sapiens
SWPAVGNCSSALR

SEQ ID NO: 348 - protein, 22 aa, Homo sapiens
VKPNPPHNLSVINSEELSSILK

SEQ ID NO: 349 - protein, 42 aa, Homo sapiens
VPMMLQSSTISYLHDSELPCQLVQMNYVGNGTVFFILPDKGK

SEQ ID NO: 350 - protein, 27 aa, Homo sapiens
VSNQTLSLFFTVLQDVPVRDLKPAIVK

SEQ ID NO: 351 - protein, 12 aa, Homo sapiens
VYSGILNQSEIK

SEQ ID NO: 352 - protein, 18 aa, Homo sapiens
WNPCLEPHRFNDTEVLQR

SEQ ID NO: 353 - protein, 27 aa, Homo sapiens
YTTFEYPNTINFSCNTGFYLNGADSAK

SEQ ID NO: 354 - protein, 14 aa, Homo sapiens
AFNSTLPTHAQHEK

SEQ ID NO: 355 - protein, 8 aa, Homo sapiens
KEDVPSER

SEQ ID NO: 356 - protein, 8 aa, Homo sapiens
KAVPYPQR

SEQ ID NO: 357 - protein, 13 aa, Homo sapiens
RFFVAPFPEVFGK

SEQ ID NO: 358 - protein, 11 aa, Homo sapiens
RYLGYLEQLLR

(19) United States
(12) Patent Application Publication
(10) Pub. No.: US 2016/0238615 A1
(43) Pub. Date: Aug. 18, 2016

(54) SOLID PHASE EXTRACTION OF GLOBAL PEPTIDES, GLYCOPEPTIDES, AND GLYCANS USING CHEMICAL IMMOBILIZATION IN A PIPETTE TIP

(71) Applicant: THE JOHNS HOPKINS UNIVERSITY, Baltimore, MD (US)

(72) Inventors: Hui ZANG, ELLICOTT CITY, MD (US); Punit SHAH, BALTIMORE, MD (US); JING CHEN, BALTIMORE, MD (US)

(73) Assignee: THE JOHNS HOPKINS UNIVERSITY, BALTIMORE, MD (US)

(21) Appl. No.: 15/024,424

(22) PCT Filed: Sep. 29, 2014
(86) PCT No.: PCT/US14/58087

§ 371 Date: Mar. 24, 2016

Related U.S. Application Data
(60) Provisional application No. 61/883,635, filed on Sep. 27, 2013.

(51) Int. Cl. G01N 33/68; G01N 35/10; G01N 35/00; B01L 3/02

(52) U.S. Cl. CPC G01N 33/6848; B01L 3/0275; G01N 33/6842; G01N 35/10; G01N 35/0099; B01L 2200/0631; B01L 2200/12; G01N 2400/00; G01N 2333/976; G01N 2333/98; G01N 2570/00; G01N 2035/1053; B01L 2300/06

(57) ABSTRACT

Pipette tips comprising aldehyde-reactive or amino-reactive chemical moieties or other chemical moieties capable of conjugating to one or more reactive groups of amino acid side chains or protein modifications and methods for preparing the tips are provided. In addition, a high throughput method for identifying proteins, glycoproteins, and glycans in a plurality of samples using the pipette tips is also provided.

### CROSS-REFERENCE

This application claims the benefit of U.S. Provisional Application No. 61/883,635, filed on Sep. 27, 2013, which application is incorporated herein by reference in its entirety.

### FEDERALLY SPONSORED RESEARCH OR DEVELOPMENT

This invention was made with government support under U01CA152813, U24CA160036, P01HL107153 and R01CA112314 awarded by the National Institutes of Health (NIH). The government has certain rights in the invention.

### BACKGROUND

Proteomics analysis is important for characterizing tissues or body fluids to gain biological and pathological insights. This could lead to the identification of disease-associated proteins as disease diagnostics or therapeutics. Glycoproteins modified by oligosaccharides are expressed as transmembrane proteins, extracellular proteins, or proteins secreted to body fluids, such as blood serum, which is an excellent source for diagnosis and monitoring of the presence and stage of many diseases (Wang et al., 2013; Zhang et al., 2013). As an easily accessible body fluid, human serum contains a large array of proteins that are derived from cells and tissues all over the body. Thus, the human serum proteome contains valuable information where biomarkers may be discovered for clinical use, e.g. CA125 for ovarian cancer and PSA for prostate cancer (Maggino and Gadducci, 2000; Schroder et al., 2007). It is considerably important to study protein glycosylation and the associated glycans for diagnostics and disease prognostics. Unlike other protein modifications, glycans attached to proteins are enormously complex. Development of the high-throughput method for extraction of peptides, glycopeptides, and glycans will facilitate proteomics, glycoproteomics, and glycomics analyses.

To analyze glycoproteins, a robust method for isolating formerly N-linked glycopeptides using solid-phase extraction of N-linked glycopeptides from glycoproteins (SPEG) has been widely used (Zhang et al., 2003). This method isolates formerly N-linked glycopeptides containing glycosylation sites for N-glycans attachments and analyzes the peptides by mass spectrometry. Human serum N-linked glycoproteome is of special interest for a number of reasons (Zhang et al, 2006; Zhou et al., 2007). First, by focusing on formerly N-linked glycopeptides, the complexity of the proteome is greatly reduced by only analyzing 1-2 N-glycosite containing peptides for each protein (Zhang et al., 2005). Second, the high abundant non-glycoproteins, e.g., albumin, which accounts for approximately 50% of proteins in human serum, are eliminated for mass spectrometry analysis. Third, glycoproteins account for most of the serum proteins that are derived from tissues where biomarkers may be identified. Fourth, aberrantly glycosylated peptides can be specifically isolated and analyzed using enrichment of glycopeptides with specific glycans (Tian et al., 2012; Li et al., 2011).

Numerous studies have been carried out using the SPEG method for cancer biomarker discovery in serum and other body fluid including breast, ovarian, lung and liver cancers (Boersema et al., 2013; Wu et al., 2013; Li et al., 2013; Sanda et al., 2013). The SPEG method includes coupling of glycoproteins to a solid support using hydrazide chemistry and removal of non-glycoproteins, proteolysis of captured glycoproteins to hydrazide with trypsin, removal of digested non-glycopeptides with washing, and specific release of N-glycopeptides using peptide-N-glycosidase F (PNGase F). This procedure provides a straightforward work flow with good protein/peptide identification and specificity. The procedure, however, requires a long processing time, such as four days (Zhang et al., 2003; Zhou et al., 2007), and is hard to scale up. In addition, the procedure releases the formerly N-linked glycopeptides containing N-glycosylation sites from their attached glycans and loses the information of glycans and total proteins from the samples where the glycopeptides are from.

### SUMMARY

In one aspect, the presently disclosed subject matter provides a pipette tip comprising an elongate body having a proximal end adapted to connect to and be in fluid communication with an outlet of a fluid dispensing device and a distal end having an opening adapted to dispense a fluid, the elongate body further comprising a fluid path between the proximal end and the distal end, wherein the fluid path comprises: (a) a first frit proximate the distal end and a second frit proximate the proximal end, and wherein the fluid path comprises a solid phase disposed between the first frit and the second frit, the solid phase comprising: (i) a chemical moiety capable of conjugating one or more glycoproteins through one or more oxidized glycans; or (ii) an amino-reactive moiety capable of conjugating one or more amino groups of one or more proteins disposed in the fluid path between the first frit and the second frit; or (iii) other chemical moieties capable of conjugating to one or more reactive groups of amino acid side chains or protein modifications disposed in the fluid path between the first frit and the second frit; or (b) a monolith-bonded aldehyde-reactive chemical moiety, a monolith-bonded amino-reactive moiety or other chemical moieties capable of conjugating to one or more reactive groups of amino acid side chains or protein modifications.

In certain aspects, the presently disclosed subject matter provides a method for preparing a pipette tip, the method comprising: (a) providing a pipette tip comprising an elongate body having a proximal end adapted to connect to and be in fluid communication with an outlet of a fluid dispensing device and a distal end having an opening adapted to dispense a fluid; and (b) forming a fluid path between the proximal end and the distal end by one of: (i) disposing a first frit proximate the distal end of the pipette tip and disposing thereon a solid phase comprising one of a chemical moiety capable of conjugating one or more glycoproteins through one or more oxidized glycans or an amino-reactive moiety or other chemical moieties capable of conjugating to one or more reactive groups of amino acid side chains or protein modifications capable of conjugating one or more amino groups of one or more proteins, and disposing a second frit proximate the proximal end of the pipette tip; or (ii) disposing a monolith-bonded aldehyde-reactive chemical moiety or a monolith-bonded amino-reactive moiety or other chemical moieties capable of conjugating to one or more reactive groups of amino acid side chains or protein modifications between the distal end and the proximal end of the pipette tip.

In particular aspects, the presently disclosed subject matter provides a kit comprising at least one presently disclosed pipette tip, wherein the kit further comprises a set of instructions for using the at least one pipette tip to isolate a biological molecule.

In more particular aspects, the presently disclosed subject matter provides a high throughput method for identifying a protein, glycoprotein, or a glycan in a plurality of samples, the method comprising: (a) providing a plurality of samples comprising at least one protein comprising at least one peptide amino group or at least one glycoprotein comprising at least one oxidized glycan or at least one reactive groups of amino acid side chains or protein modifications; (b) disposing the plurality of samples in a plurality of pipette tips, wherein each pipette tip comprises an elongate body having a proximal end adapted to connect to and be in fluid communication with an outlet of a fluid dispensing device and a distal end having an opening adapted to dispense a fluid, the elongate body further comprising a fluid path between the proximal end and the distal end, wherein the fluid path comprises: (i) a first frit proximate the distal end and a second frit proximate the proximal end, and wherein the fluid path comprises a solid phase disposed between the first frit and the second frit, the solid phase comprising a chemical moiety capable of conjugating one or more glycoproteins through one or more oxidized glycans or an amino-reactive moiety capable of conjugating one or more amino groups or other chemical moieties capable of conjugating to one or more reactive groups of amino acid side chains or protein modifications of one or more proteins disposed in the fluid path between the first frit and the second frit; or (ii) a monolith-bonded aldehyde-reactive chemical moiety or a monolith-bonded amino-reactive moiety or other chemical moieties capable of conjugating to one or more reactive groups of amino acid side chains or protein modifications; (c) conjugating the at least one protein or at least one glycoprotein comprising the plurality of samples to the solid phase chemical moieties capable of conjugating to one or more reactive groups of amino acid side chains or protein modifications or the monolith-bonded aldehyde-reactive chemical moiety or amino-reactive moiety or other chemical moieties capable of conjugating to one or more reactive groups of amino acid side chains or protein modifications; (d) cleaving the at least one protein thereby releasing at least one peptide fragment or releasing the at least one former glycopeptide fragment or glycan; and (e) analyzing the at least one peptide, glycan or the at least one former glycopeptide fragment to identify the protein, glycan from which the at least one peptide and glycan fragment was derived or to identify the glycoprotein from which the former glycopeptide fragment was derived; and wherein at least one step of the method is automated.

Certain aspects of the presently disclosed subject matter having been stated hereinabove, which are addressed in whole or in part by the presently disclosed subject matter, other aspects will become evident as the description proceeds when taken in connection with the accompanying Examples and Figures as best described herein below.

### BRIEF DESCRIPTION OF THE FIGURES

Having thus described the presently disclosed subject matter in general terms, reference will now be made to the accompanying Figures, which are not necessarily drawn to scale, and wherein:

FIGS. 1A-1B show (A) an embodiment of the workflow of the presently disclosed formerly N-linked glycopeptide isolation using a hydrazide tip; and (B) a representative embodiment of the presently disclosed pipette tip comprising a aldehyde-reactive hydrazide moiety;

FIGS. 2A-2D show an experiment for the determination of time required for coupling, trypsin digestion and PNGase F release on a tip: (A) coupling time course: oxidized bovine fetuin was pipetted through a hydrazide tip. Concentration of protein uncoupled was measured at various time points; (B) digestion time course: fetuin conjugated to a hydrazide tip was subjected to trypsin digestion. Concentration of non-glycopeptide released from glycoprotein conjugated on hydrazide tip was measured at various time points; (C) fetuin glycopeptides conjugated to hydrazide tip through N-linked glycans were released by PNGase F. Peptide released was measured at various time points; and (D) a representative MALDI spectra of formerly N-linked glycopeptides from fetuin; (S): Signal to Noise ratio of each peak;

FIGS. 3A-3B show Venn diagrams comparing the serum N-linked glycopeptide identified from three LC-MS/MS replicates and three isolation replicates. The diagram illustrates similarities and differences in the peptides identified in (A) each of the three isolation replicates and (B) each of the three LC-MS/MS replicates (Injection 1=333, Injection 2=341, Injection 3=332) by Proteome Discoverer software searches (Thermo Fisher Scientific, Waltham, Mass.) of MS/MS data;

FIGS. 4A-4B show liquid chromatography profiles of serum N-linked glycopeptide from three LC-MS/MS replicates and three isolation replicates. The raw files of (A) the three LC-MS/MS replicates or (B) the three isolation replicates were displayed in Xcalibur and the base peak profiles were overlaid for visualization of LC variability;

FIGS. 5A-5C show an embodiment of the scheme for Chemical Immobilization of Proteins for Peptide Extraction (CIPPE). Proteins are conjugated onto the solid support. Unbound compounds including OCT are washed away. Peptides are released from the solid support using proteolysis and analyzed using LC-MS/MS: (A, B) an embodiment of the workflow of the presently disclosed immobilization of proteins on a solid phase in a tip and releasing of peptides for global proteomics analysis from proteins immobilized in an amino-reactive resin in a tip; and (C) an embodiment of the workflow of the presently disclosed conjugation of proteins on a solid phase in a tip and releasing glycans from glycoproteins for glycomic analysis;

FIGS. 6A-6C show mass spectrometric detection of the tryptic peptides from HSA with and without OCT: A) a representative electrospray ionization (ESI) spectrum of the tryptic peptides from OCT contaminated HSA digested in solution; B) a representative mass spectrum of OCT contaminated HSA after OCT removal using CIPPE; and C) a representative mass spectrum of clean HSA digested in solution;

FIG. 7 shows a schematic diagram for the relative quantification to study the impact of OCT on tissue samples using CIPPE method. Mouse kidney was split into two pieces. One was embedded in OCT, and second was directly frozen at −80° C. Proteins were extracted from two OCT-embedded tissues and one frozen tissue using CIPPE. Peptides were labeled with iTRAQ tags and labeled peptides were combined. Peptide sample was then divided into two fractions and 90% of sample was used for glycopeptide extraction using the SPEG method. The iTRAQ labeled tryptic peptides and glycopeptides were analyzed using LC-MSMS;

FIGS. 8A-8B show quantitative analysis of proteins and glycoproteins isolated from OCT-embedded tissues using CIPPE. Scatter plot represents proteome (A) and glycoproteome (B). The two channels 114 and 115 were quantitative analysis of two OCT embedded tissues using CIPPE. The intensities observed for peptides in channels 114 and 115 were plotted in X axis and Y axis respectively for each PSM. Scatter plot represents quantitative linearity between reporter ion groups, the sample and the reporter ion intensity scatter plot are grouped around a 45° line indicating symmetric distribution of fold change across the scatter plot;

FIGS. 9A-9D show quantitative analysis of proteins and glycoproteins form OCT-embedded tissue and frozen tissue: (A) scatter plot representing proteome; (B) scatter plot representing the glycoproteome. Channel 114 represents OCT embedded tissue and 116 represents frozen tissue. The intensities observed for peptides in channels 114 and 116 are plotted in X axis and Y axis respectively. Scatter plot represents quantitative linearity between reporter ion groups, the sample and the reporter ion intensity scatter plot are grouped around a 45° line. The data shows symmetric distribution of fold changes across the scatter plot; (C) global proteomics plotted protein ratio log₂(116/114) in Y axis and log₂(115/114) in X axis; and (D) glycoprotein plotted similarly. The results are centered on origin indicating high quantitative similarity between OCT embedded tissue and frozen tissue analysis using CIPPE;

FIGS. 10A-10B show representative MALDI spectra of released tryptic global peptides released from casein immobilized to solid phase by reductive amination with a mass range of 500-4000 using an embodiment of the tube digestion method and the tip method. K.EDVPSER (SEQ ID NO:355); K.AVPYPQR (SEQ ID NO:356) is a peptide from beta casein;

FIGS. 11A-11B show representative MALDI spectra of released tryptic peptides from casein immobilized to solid phase in tip with a mass range of 900-1700 using an embodiment of the tube digestion method and the tip method. R.FFVAPFPEVFGK (SEQ ID NO:357) and R.YLGYLEQLLR (SEQ ID NO:358) are peptides from alpha-S1-casein;

FIGS. 12A-12B show an embodiment of a workflow scheme of N-glycan isolation: (A) scheme of GIG isolation; and (B) scheme of GIG isolation using aldehyde tips. Proteins from samples were first immobilized onto beads/tip columns. Sialic acid was then modified with p-toluidine. The beads/tips were subsequently washed extensively in 1% formic acid, 1M NaCl, 10% acetonitrile, and water. N-glycans were finally released with PNGase F;

FIGS. 13A-13B show an embodiment of aldehyde tips: (A) a photograph of a unpacked and packed aldehyde tip; and (B) a photograph of 96-well aldehyde tips loaded in a robotic liquid handling system for automated glycan extraction;

FIGS. 14A-14B show optimization of reaction time for coupling and PNGase F release: (A) serum proteins were slowly pipetted through aldehyde tips for various amount of time. Complete coupling was achieved after 30 min reaction; and (B) after extensive washing and sialic acid labeling, the N-glycans from serum proteins were released from the aldehyde tips with PNGase F for various times. N-glycan was still releasing after 2 hours;

FIG. 15 shows MALDI-MS profiles of serum N-glycans isolated with aldehyde tips;

FIG. 16 shows representative MALDI profiles of three isolations of N-glycan from human serum. N-glycans from three human serum samples (20 μL each) were isolated in parallel using the aldehyde tips with a robotic liquid handling system;

FIG. 17 shows representative reproducibility of N-glycan isolation. Glycans shown in FIG. 16 were quantified;

FIG. 18 shows an embodiment of the workflow of using p-toluidine to modify the acid component of proteins and sialylated glycans and quantifying of glycans and glycopeptides using MALDI-MS;

FIGS. 19A-19C show N-glycans identified and quantified from SW1990 Cells using the method shown in FIG. 18: (A) heavy and light labeled cell mix; (B) light labeled cell mix, no ManNAc treatment; and (C) heavy labeled ManNAc treated cell;

FIG. 20 shows an embodiment of the workflow for glycopeptide analysis using basic reverse phase fractionation;

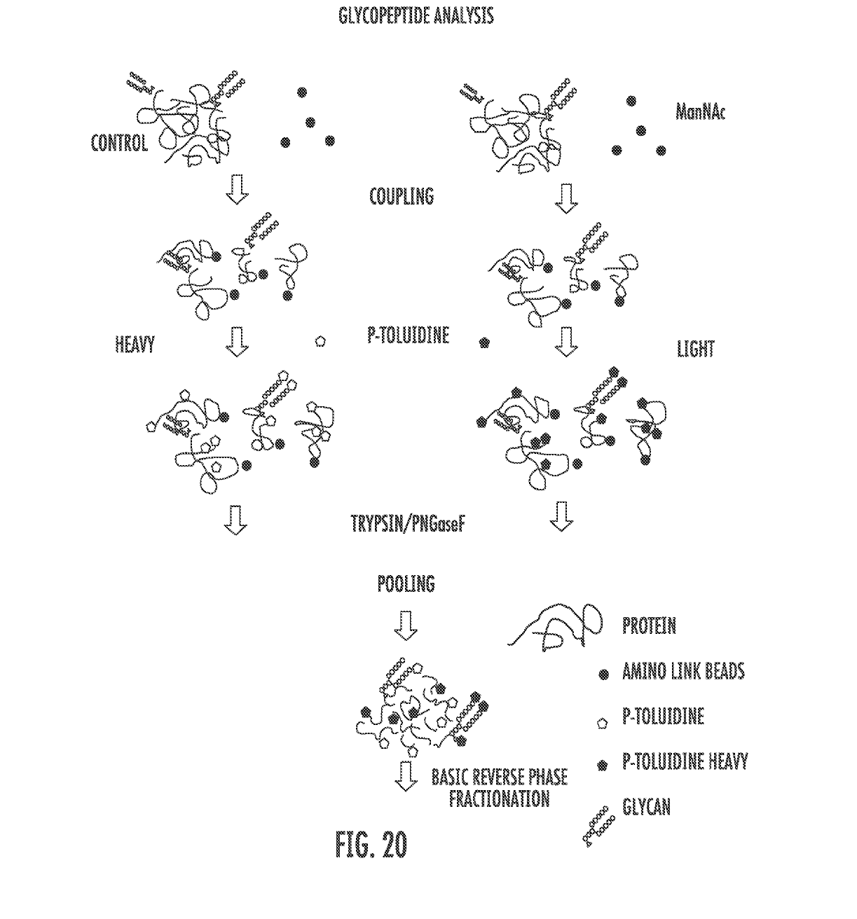

FIG. 21 shows an embodiment of the workflow of the presently disclosed conjugation of proteins on a solid phase in a tip. Sample preparation including labeling was automated using liquid handling robotic systems;

FIG. 22 shows results from the method shown in FIG. 20; and

FIG. 23 shows quantitation of AFNSTLPTHAQHEK (SEQ ID NO: 354) CD44 glycopeptide with triattenary sialylated peptide.

### DETAILED DESCRIPTION

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

The glycoproteome contains valuable information, such as biomarkers that may be discovered for disease diagnosis and monitoring. With the ever increasing performances of mass spectrometers, the emphasis is shifting to the sample preparation step for better throughput and reproducibility. In addition, a greater than ever number of samples are being processed and subjected to mass spectrometry analysis, calling for automation for high throughput sample preparation. Automation can minimize variability due to human errors, provide greater consistency and reduce sample preparation time and effort. Therefore, to meet the pressing need in the mass spectrometry field, the presently disclosed subject matter provides a novel pipette tip, such as a hydrazide tip, and methods for an integrated workflow of glycopolypeptide or polypeptide isolation using the tips. In some embodiments, with the presently disclosed tips and methods thereof, the processing time is decreased to less than 8 hours. In other embodiments, glycoprotein or protein isolation can be automated using a liquid handling robot system.

### I. Pipette Tips

A. Pipette Tips

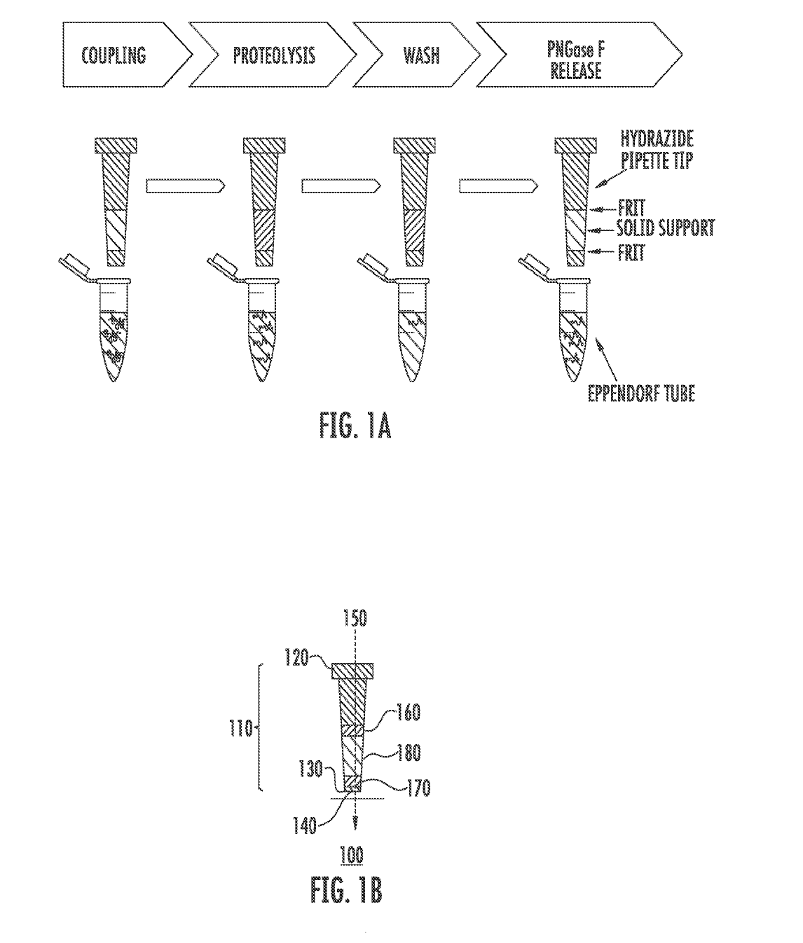

FIG. 1A shows, in some embodiments, the workflow of the presently disclosed formerly N-linked glycopeptide isolation using a hydrazide tip.

Referring now to FIG. 1B, in some embodiments, the presently disclosed subject matter provides a pipette tip 100, which includes elongate body 110 having proximal end 120 adapted to connect to and be in fluid communication with an outlet of a fluid dispensing device (not shown) and distal end 130 having opening 140 adapted to dispense a fluid, the elongate body 110 further comprising fluid path 150 between proximal end 120 and distal end 130, wherein fluid path 150 comprises first frit 170 proximate distal end 130 and second frit 160 proximate distal end 120, and wherein fluid path 150 comprises solid phase 180 disposed between first frit 170 and second frit 160, wherein the solid phase 180 comprises: (i) a chemical moiety capable of conjugating one or more glycoproteins through one or more oxidized glycans; or (ii) an amino-reactive moiety capable of conjugating one or more amino groups of one or more proteins disposed in the fluid path 150 between the first frit 170 and the second frit 160; or (iii) other chemical moieties capable of conjugating to one or more reactive groups of amino acid side chains or protein modifications disposed in the fluid path 150 between the first frit 170 and the second frit 160.

The pipette tip can be any kind, shape, or size, depending on the amount of chemical or amino-reactive moiety required, the kind of automated apparatus used, and the like for the particular presently disclosed methods. A person with ordinary skill in the art will appreciate that standard sizes of pipette tips are commercially available, such as from 50 μL to 1000 μL. In a preferred embodiment, the pipette tips used are meant for automated pipetting functions so that the hydrazide pipette tips can be used for high throughput methods.

In some embodiments, the presently disclosed subject matter provides a pipette tip comprising an elongate body having a proximal end adapted to connect to and be in fluid communication with an outlet of a fluid dispensing device and a distal end having an opening adapted to dispense a fluid, the elongate body further comprising a fluid path between the proximal end and the distal end, wherein the fluid path comprises: (a) a first frit proximate the distal end and a second frit proximate the proximal end, and wherein the fluid path comprises a solid phase disposed between the first frit and the second frit, the solid phase comprising: (i) a chemical moiety capable of conjugating one or more glycoproteins through one or more oxidized glycans; or (ii) an amino-reactive moiety capable of conjugating one or more amino groups of one or more proteins disposed in the fluid path between the first frit and the second frit; or (iii) other chemical moieties capable of conjugating to one or more reactive groups of amino acid side chains or protein modifications disposed in the fluid path between the first frit and the second frit; or (b) a monolith-bonded aldehyde-reactive chemical moiety, a monolith-bonded amino-reactive moiety or other chemical moieties capable of conjugating to one or more reactive groups of amino acid side chains or protein modifications.

The solid phase comprising a chemical moiety, such as a hydrazide moiety or an amino-reactive moiety or other chemical moieties capable of conjugating to one or more reactive groups of amino acid side chains or protein modifications, can be, for example, a bead, resin, slurry, monolith, membrane or disk, or any generally solid phase material suitable for the presently disclosed methods. An advantage of using a solid phase is that it allows extensive washing to remove undesired molecules. Another advantage of the solid phase is that it allows further manipulation of the sample molecules without the need for additional purification steps that can result in loss of sample molecules. In some embodiments, the chemical moiety is selected from the group consisting of one or more aldehyde-reactive hydrazide beads/resin/monolith or amino-reactive beads/resin/monolith or beads/resin/monolith with other chemical moieties capable of conjugating to one or more reactive groups of amino acid side chains or protein modifications. In other embodiments, the aldehyde-reactive chemical moiety is used for glycan conjugation and the amino-reactive moiety is used for polypeptide conjugation or other chemical moieties capable of conjugating to one or more reactive groups of amino acid side chains or protein modifications.

Frits, also known as filters, are available in a wide variety of porous plastics such as polyethylene (PE), polytetrafluoroethylene (PTFE), oleophobic-treated PTFE, functionalized and surface-modified porous materials, bio-activated porous media, and the like. As used herein, in some embodiments, the frits hold the solid phase comprising an aldehyde-reactive chemical moiety or amino-reactive moiety or other chemical moieties capable of conjugating to one or more reactive groups of amino acid side chains or protein modifications in place and help protect the medium from running dry under buffer flow.

In some embodiments, the pipette tip comprises hydrazide resin. In other embodiments, the hydrazide resin has a particle size ranging from about 40 micrometers to about 60 micrometers. In further embodiments, the particle size range of the hydrazide resin is about 75 micrometers to about 300 micrometers. In still further embodiments, the first frit and the second frit have a pore size ranging from about 15 to about 45 microns.

In some embodiments, the pipette tip comprises more than two frits, such as 3, 4, 5, or more frits.

B. Methods for Preparing Pipette Tips

Referring again to FIG. 1B, in some embodiments, the presently disclosed subject matter provides methods for preparing a pipette tip 100. In some embodiments, the method comprises pushing a first frit 170 into elongate body 110, adding a solid phase 180 to the elongate body 110 from the proximal end 120, pushing a second frit 160 through the proximal end 120 to secure the solid phase 180 between the two frits 160 and 170, wherein adding a solid phase 180 to the elongate body 110 comprises forming a fluid path 150 between the proximal end 120 and the distal end 130. Forming a fluid path 150 comprises one of: (i) disposing a first frit 170 proximate the distal end 130 of the pipette tip 100 and disposing thereon a solid phase 180 comprising one of a chemical moiety capable of conjugating one or more glycoproteins through one or more oxidized glycans or an amino-reactive moiety or other chemical moieties capable of conjugating to one or more reactive groups of amino acid side chains or protein modifications capable of conjugating one or more amino groups of one or more proteins, and disposing a second frit 160 proximate the proximal end 120 of the pipette tip 100; or (ii) disposing a monolith-bonded aldehyde-reactive chemical moiety or a monolith-bonded amino-reactive moiety or other chemical moieties capable of conjugating to one or more reactive groups of amino acid side chains or protein modifications between the distal end 130 and the proximal end 120 of the pipette tip 100.

In some embodiments, the presently disclosed subject matter provides a method for preparing a pipette tip, the method comprising: (a) providing a pipette tip comprising an elongate body having a proximal end adapted to connect to and be in fluid communication with an outlet of a fluid dispensing device and a distal end having an opening adapted to dispense a fluid; and (b) forming a fluid path between the proximal end and the distal end by one of: (i) disposing a first frit proximate the distal end of the pipette tip and disposing thereon a solid phase comprising one of a chemical moiety capable of conjugating one or more glycoproteins through one or more oxidized glycans or an amino-reactive moiety or other chemical moieties capable of conjugating to one or more reactive groups of amino acid side chains or protein modifications capable of conjugating one or more amino groups of one or more proteins, and disposing a second frit proximate the proximal end of the pipette tip; or (ii) disposing a monolith-bonded aldehyde-reactive chemical moiety or a monolith-bonded amino-reactive moiety or other chemical moieties capable of conjugating to one or more reactive groups of amino acid side chains or protein modifications between the distal end and the proximal end of the pipette tip. In some embodiments, the chemical moiety comprises an aldehyde-reactive moiety. In other embodiments, the first frit and the second frit have a pore size ranging from about 15 to about 45 microns. In still other embodiments, the methods further comprise washing the solid phase after the solid phase is disposed on the first frit. In further embodiments, the methods further comprise washing the solid phase with a liquid selected from the group consisting of water and a buffer.

Pushing the frits into the pipette tip can be performed with any tool that will allow the frit to be placed into the pipette tip, such as a tweezer, a needle, and the like. Likewise, there are different ways that the solid phase can be added to the pipette tip, such as using a pipetter and another pipette tip, a dropper, a micro capillary pipette, and the like.

C. Kits Comprising Pipette Tips

In general, a presently disclosed kit contains some or all of the components, reagents, supplies, and the like to practice a method according to the presently disclosed subject matter. In some embodiments, the presently disclosed subject matter provides a kit comprising at least one presently disclosed pipette tip, wherein the kit further comprises a set of instructions for using the at least one pipette tip to isolate a biological molecule.

### II. Methods for Identifying Proteins and Glycoproteins

Protein glycosylation has long been recognized as a very common post-translational modification. Carbohydrates are linked to serine or threonine residues (O-linked glycosylation) or to asparagine residues (N-linked glycosylation). Protein glycosylation, and in particular N-linked glycosylation, is prevalent in proteins destined for extracellular environments. These include proteins on the extracellular side of the plasma membrane, secreted proteins, and proteins contained in body fluids, for example, blood serum, cerebrospinal fluid, urine, breast milk, saliva, lung lavage fluid, pancreatic juice, and the like. In some embodiments, the plurality of samples is selected from the group consisting of a body fluid, a secreted protein, and a cell surface protein.

The presently disclosed subject matter provides methods for quantitative profiling of glycoproteins and glycopeptides on a proteome-wide scale. The methods allow the identification and quantification of glycoproteins in a complex sample and determination of the sites of glycosylation. The methods can be used to determine changes in the abundance of glycoproteins and changes in the state of glycosylation at individual glycosylation sites on those glycoproteins that occur in response to perturbations of biological systems and organisms in health and disease.

The presently disclosed methods can be used to purify glycosylated proteins or peptides and identify and quantify the glycosylation sites. In some embodiments, because the methods can be directed to isolating glycoproteins, the methods also reduce the complexity of analysis since many proteins and fragments of glycoproteins do not contain carbohydrate. This can simplify the analysis of complex biological samples such as serum. The methods are advantageous for the determination of protein glycosylation in glycome studies and can be used to isolate and identify glycoproteins from cell membrane or body fluids to determine specific glycoprotein changes related to certain disease states or cancer. The methods can be used for detecting quantitative changes in protein samples containing glycoproteins and to detect their extent of glycosylation. The methods can be used for identifying oligosaccharides in samples. The methods are applicable for the identification and/or characterization of diagnostic biomarkers, immunotherapy, or other diagnostic or therapeutic applications. The methods can also be used to evaluate the effectiveness of drugs during drug development, optimal dosing, toxicology, drug targeting, and related therapeutic applications.

The presently disclosed tips and methods can be used to identify many different types of glycoproteins, glycans or proteins. These include mucins, collagens, antibodies, molecules of the major histocompatibility complex (MHC), viral glycoproteins, hormones, transport molecules, such as transferrin and ceruloplasmin, enzymes, various proteins involved in cell interactions with other cells, a virus, a bacterium, or a hormone, plasma proteins, calnexin, calreticulin, fetuin, casein, proteins involved in the regulation of development, specific glycoproteins on the surface membranes of platelets, and the like.

In some embodiments, the presently disclosed subject matter provides a high throughput method for identifying a protein, glycoprotein, or a glycan in a plurality of samples, the method comprising: (a) providing a plurality of samples comprising at least one protein comprising at least one peptide amino group or at least one glycoprotein comprising at least one oxidized glycan or at least one reactive groups of amino acid side chains or protein modifications; (b) disposing the plurality of samples in a plurality of pipette tips, wherein each pipette tip comprises an elongate body having a proximal end adapted to connect to and be in fluid communication with an outlet of a fluid dispensing device and a distal end having an opening adapted to dispense a fluid, the elongate body further comprising a fluid path between the proximal end and the distal end, wherein the fluid path comprises: (i) a first frit proximate the distal end and a second frit proximate the proximal end, and wherein the fluid path comprises a solid phase disposed between the first frit and the second frit, the solid phase comprising a chemical moiety capable of conjugating one or more glycoproteins through one or more oxidized glycans or an amino-reactive moiety capable of conjugating one or more amino groups or other chemical moieties capable of conjugating to one or more reactive groups of amino acid side chains or protein modifications of one or more proteins disposed in the fluid path between the first frit and the second frit; or (ii) a monolith-bonded aldehyde-reactive chemical moiety or a monolith-bonded amino-reactive moiety or other chemical moieties capable of conjugating to one or more reactive groups of amino acid side chains or protein modifications; (c) conjugating the at least one protein or at least one glycoprotein comprising the plurality of samples to the solid phase chemical moieties capable of conjugating to one or more reactive groups of amino acid side chains or protein modifications or the monolith-bonded aldehyde-reactive chemical moiety or amino-reactive moiety or other chemical moieties capable of conjugating to one or more reactive groups of amino acid side chains or protein modifications; (d) cleaving the at least one protein thereby releasing at least one peptide fragment or releasing the at least one former glycopeptide fragment or glycan; and (e) analyzing the at least one peptide, glycan or the at least one former glycopeptide fragment to identify the protein, glycan from which the at least one peptide and glycan fragment was derived or to identify the glycoprotein from which the former glycopeptide fragment was derived; and wherein at least one step of the method is automated. In other embodiments, the chemical moiety comprises a hydrazide moiety. In still other embodiments, the hydrazide moiety comprises a hydrazide resin.

The use of biological fluids, such as a body fluid as a sample source, is particularly useful for the presently disclosed methods. Biological fluid specimens are generally readily accessible and available in relatively large quantities for clinical analysis. Biological fluids can be used to analyze diagnostic and prognostic markers for various diseases. In addition to ready accessibility, body fluid specimens do not require any prior knowledge of the specific organ or the specific site in an organ that might be affected by disease. Because body fluids, in particular blood, are in contact with numerous body organs, body fluids “pick up” molecular signatures indicating pathology due to secretion or cell lysis associated with a pathological condition. Body fluids also pick up molecular signatures that are suitable for evaluating drug dosage, drug targets and/or toxic effects, as disclosed herein. In some embodiments, the plurality of samples is selected from the group consisting of samples comprising a body fluid, a secreted protein, and a cell surface protein.

The carbohydrate moieties of a glycoprotein are chemically or enzymatically modified to generate a reactive group that can be selectively bound to a solid support or solid phase having a corresponding reactive group. In some embodiments, at least one glycoprotein is oxidized with periodate. For example, the cis-diol groups of carbohydrates in glycoproteins can be oxidized by periodate oxidation to give a di-aldehyde, which reacts with a hydrazide moiety to form covalent hydrazone bonds. The hydroxyl groups of a carbohydrate can also be derivatized by epoxides or oxiranes, alkyl halogen, carbonyldiimidazoles, N,N′-disuccinimidyl carbonates, N-hydroxycuccinimidyl chloroformates, and the like. The hydroxyl groups of a carbohydrate can also be oxidized by enzymes to create reactive groups such as aldehyde groups. For example, galactose oxidase oxidizes terminal galactose or N-acetyl-D-galactose residues to form C-6 aldehyde groups. These derivatized groups can be conjugated to hydrazide-containing moieties.

In some embodiments, after being oxidized, at least one glycoprotein or protein is removed from the oxidation buffer and disposed in a coupling buffer. In other embodiments, the coupling buffer is a high salt and acidic pH buffer. In still other embodiments, the presently disclosed methods further comprise adding aniline to the coupling buffer. Aniline can be used as a catalyst to improve the reaction rate between aldehyde and hydrazide groups (Zeng et al., 2009; Dirksen et al., 2010).

After the samples are oxidized, they are added to the pipette tips for immobilization of the glycoproteins and/or the proteins. In some embodiments, the methods further comprise washing the at least one protein or the at least one glycoprotein with a urea buffer before being reduced.

If desired, the bound glycoproteins or proteins can be denatured and optionally reduced. Denaturing and/or reducing the bound glycoproteins or proteins can be useful prior to cleavage of the glycoproteins or proteins, in particular protease cleavage, because this allows access to protease cleavage sites that can be masked in the native form of the glycoproteins or proteins. The bound glycoproteins or proteins can be denatured with detergents and/or chaotropic agents. Reducing agents such as β-mercaptoethanol, dithiothreitol, tris-carboxyethylphosphine (TCEP), and the like, can also be used, if desired. The binding of the glycoproteins or proteins to a solid phase allows the denaturation step to be carried out followed by extensive washing to remove denaturants that could inhibit the enzymatic or chemical cleavage reactions. The use of denaturants and/or reducing agents can also be used to dissociate protein complexes in which non-glycosylated proteins form complexes with bound glycoproteins. Thus, the use of these agents can be used to increase the specificity for glycoproteins by washing away non-glycosylated proteins from the solid phase. In some embodiments, the at least one protein or the at least one glycoprotein is reduced with tris(2-carboxyethyl) phosphine (TCEP).

In some embodiments, at least one protein or glycoprotein is alkylated. In other embodiments, the at least one protein or the at least one glycoprotein is alkylated with iodoacetamide (IAA). In still other embodiments, the methods further comprise washing the at least one alkylated protein or the at least one alkylated glycoprotein with a urea buffer before being cleaved.

The bound glycoproteins or proteins can be cleaved into peptide fragments to facilitate analysis. Thus, a protein molecule can be enzymatically cleaved with one or more proteases into peptide fragments. Exemplary proteases useful for cleaving polypeptides include trypsin, chymotrypsin, pepsin, papain, Staphylococcus aureus (V8) protease, Submaxillaris protease, bromelain, thermolysin, and the like. In certain applications, proteases having cleavage specificities that cleave at fewer sites, such as sequence-specific proteases having specificity for a sequence rather than a single amino acid, can also be used, if desired. Polypeptides can also be cleaved chemically, for example, using CNBr, acid or other chemical reagents. One skilled in the art can readily determine appropriate conditions for cleavage to achieve a desired efficiency of peptide cleavage. In some embodiments, the at least one alkylated protein or the at least one alkylated glycoprotein is cleaved with trypsin.

However, in other embodiments, cleavage of the bound glycoproteins or proteins is not required, in particular where the bound glycoprotein is relatively small and contains a single glycosylation site. Furthermore, the cleavage reaction can be carried out after binding of glycoproteins to the solid phase, allowing characterization of non-glycosylated peptide fragments derived from the bound glycoprotein. Alternatively, the cleavage reaction can be carried out prior to addition of the glycoproteins to the solid phase. One skilled in the art can readily determine the desirability of cleaving the sample polypeptides and an appropriate point to perform the cleavage reaction, as needed for a particular application.

In some embodiments, cleaving the at least one alkylated glycoprotein comprising at least one oxidized glycan occurs by enzymatic reaction if the at least one oxidized glycan is an N-glycan or by chemical reaction if the at least one oxidized glycan is an O-glycan. In other embodiments, cleaving of the at least one alkylated protein occurs by using a protease or a chemical. In still other embodiments, cleaving of the at least one alkylated protein leaves at least one glycopeptide on the solid phase or monolith. In further embodiments, before releasing the at least one former glycopeptide fragment, the solid phase or monolith is washed to remove the non-glycosylated peptide fragments.

In some embodiments, the at least one former glycopeptide fragment is released from the solid phase or monolith with a glycosidase or chemicals. In other embodiments, the glycosidase is selected from the group consisting of an N-glycosidase and a β-elimination. In still other embodiments, the N-glycosidase is peptide-N-glycosidase F (PNGase F). In further embodiments, at least one former glycopeptide fragment is released from the solid phase using a chemical cleavage.

In some embodiments, the glycoproteins or proteins are isotopically labeled, for example, at the amino or carboxyl termini to allow the quantities of glycoproteins or proteins from different biological samples to be compared.

After isolating the glycoproteins, glycans or proteins from a sample and cleaving the glycoprotein or protein into fragments, the former glycopeptide, glycan or peptide fragments are released from the solid phase and the released former glycopeptide, glycan or peptide fragments are identified and/or quantified. A particularly useful method for analysis of the released glycopeptide or peptide fragments is mass spectrometry. A variety of mass spectrometry systems can be employed in the methods of the invention for identifying and/or quantifying a sample molecule such as a released glycopeptide or peptide fragment. Mass analyzers with high mass accuracy, high sensitivity and high resolution include, but are not limited to, ion trap, triple quadrupole, and time-of-flight, quadrupole time-of-flight mass spectrometers and Fourier transform ion cyclotron mass analyzers (FT-ICR-MS). Mass spectrometers are typically equipped with matrix-assisted laser desorption (MALDI) and electrospray ionization (ESI) ion sources, although other methods of peptide ionization can also be used. In ion trap MS, analytes are ionized by ESI or MALDI and then put into an ion trap. Trapped ions can then be separately analyzed by MS upon selective release from the ion trap. Fragments can also be generated in the ion trap and analyzed. Sample molecules such as released glycopeptide or peptide fragments can be analyzed, for example, by single stage mass spectrometry with a MALDI-TOF or ESI-TOF system. Methods of mass spectrometry analysis are well known to those skilled in the art. In some embodiments, analyzing of the at least one glycopeptide fragment or the at least one former peptide fragment is done by mass spectrometry.

Once a peptide is analyzed by mass spectrometry, for example, the resulting CID spectrum can be compared to databases for the determination of the identity of the isolated glycopeptide or peptide. In particular, it is possible that one or a few peptide fragments can be used to identify a parent polypeptide from which the fragments were derived if the peptides provide a unique signature for the parent polypeptide. Thus, identification of a single glycopeptide, alone or in combination with knowledge of the site of glycosylation, can be used to identify a parent glycopolypeptide from which the glycopeptide fragments were derived. Further information can be obtained by analyzing the nature of the attached tag and the presence of the consensus sequence motif for carbohydrate attachment. For example, if peptides are modified with an N-terminal tag, each released glycopeptide or peptide has the specific N-terminal tag, which can be recognized in the fragment ion series of the CID spectra. Furthermore, the presence of a known sequence motif that is found, for example, in N-linked carbohydrate-containing peptides, that is, the consensus sequence NXS/T, can be used as a constraint in database searching of N-glycosylated peptides.

In addition, the identity of the parent glycopolypeptide or polypeptide can be determined by analysis of various characteristics associated with the peptide, for example, its resolution on various chromatographic media or using various fractionation methods. These empirically determined characteristics can be compared to a database of characteristics that uniquely identify a parent polypeptide, which defines a peptide tag.

In some embodiments, the method is automated, which allows many samples to be analyzed at the same time. Automated systems for testing or analyzing many samples simultaneously are known in the art. In other embodiments, the method further comprises the use of a liquid handling robot system.

### III. General Definitions

Although specific terms are employed herein, they are used in a generic and descriptive sense only and not for purposes of limitation. Unless otherwise defined, all technical and scientific terms used herein have the same meaning as commonly understood by one of ordinary skill in the art to which this presently described subject matter belongs.

As used herein, the term “polypeptide” or “protein” refers to a peptide or polypeptide of two or more amino acids. A polypeptide can also be modified by naturally occurring modifications such as post-translational modifications, including phosphorylation, fatty acylation, prenylation, sulfation, hydroxylation, acetylation, addition of carbohydrate, addition of prosthetic groups or cofactors, formation of disulfide bonds, proteolysis, assembly into macromolecular complexes, and the like. A “peptide fragment” is a peptide of two or more amino acids, generally derived from a larger polypeptide.

As used herein, a “glycopolypeptide”, “glycopeptide” or “glycoprotein” refers to a polypeptide that contains a covalently bound carbohydrate group in the intact glycoproteins and could be released free of glycans from the glycoproteins before mass spectrometric analysis. The carbohydrate can be a monosaccharide, oligosaccharide or polysaccharide. Proteoglycans are included within the meaning of “glycopolypeptide.” A glycopolypeptide can additionally contain other post-translational modifications. A “glycopeptide” refers to a peptide that comprises a covalently bound carbohydrate. A “glycopeptide fragment” refers to a peptide fragment resulting from enzymatic or chemical cleavage of a larger polypeptide in which the peptide fragment retains covalently bound carbohydrate. It is understood that a glycopeptide fragment or peptide fragment refers to the peptides that result from a particular cleavage reaction, regardless of whether the resulting peptide was present before or after the cleavage reaction. Thus, a peptide that does not contain a cleavage site will be present after the cleavage reaction and is considered to be a peptide fragment resulting from that particular cleavage reaction. For example, if bound glycopeptides are cleaved, the resulting cleavage products retaining bound carbohydrate are considered to be glycopeptide fragments. The glycosylated fragments can remain bound to the solid phase, and such bound glycopeptide fragments are considered to include those fragments that were not cleaved due to the absence of a cleavage site.

As disclosed herein, a glycopolypeptide or glycopeptide can be processed such that the carbohydrate is removed from the parent glycopolypeptide. It is understood that such an originally glycosylated polypeptide is still referred to herein as a glycopolypeptide or glycopeptide even if the carbohydrate is removed enzymatically and/or chemically. Thus, a glycopolypeptide or glycopeptide can refer to a glycosylated or de-glycosylated form of a polypeptide. A glycopolypeptide or glycopeptide from which the carbohydrate is removed is referred to as the de-glycosylated form of a polypeptide whereas a glycopolypeptide or glycopeptide which retains its carbohydrate is referred to as the glycosylated form of a polypeptide.

As used herein, the term “glycan” refers to a polysaccharide or oligosaccharide. Glycan may also be used to refer to the carbohydrate portion of a glycoconjugate, such as a glycoprotein, glycolipid, or a proteoglycan. As used herein, an “oxidized glycan” is a polysaccharide or an oligosaccharide that has been oxidized.

As used herein, a “hydrazide moiety” is a moiety comprising an acyl derivative of hydrazine.

As used herein, the term “amino-reactive moiety” is a moiety that can conjugate the amino groups of proteins.

As used herein, the term “aldehyde-reactive chemical moiety” is a moiety that can conjugate the aldehyde of a glycan.

As used herein, the term “monolith” is intended to mean a separation media that generally does not contain interparticular voids. As a result, the mobile phase flows through the stationary phase.

As used herein, the term “sample” is intended to mean any biological fluid, cell, tissue, organ or portion thereof, which includes one or more different molecules such as nucleic acids, polypeptides, or small molecules. A sample can be a tissue section obtained by biopsy, or cells that are placed in or adapted to tissue culture. A sample can also be a biological fluid specimen such as blood, serum or plasma, cerebrospinal fluid, urine, saliva, seminal plasma, pancreatic juice, breast milk, lung lavage, and the like. A sample can additionally be a cell extract from any species, including prokaryotic and eukaryotic cells as well as viruses. A tissue or biological fluid specimen can be further fractionated, if desired, to a fraction containing particular cell types.

As used herein, a “polypeptide sample” refers to a sample containing two or more different polypeptides. A polypeptide sample can include tens, hundreds, or even thousands or more different polypeptides. A polypeptide sample can also include non-protein molecules so long as the sample contains polypeptides. A polypeptide sample can be a whole cell or tissue extract or can be a biological fluid. Furthermore, a polypeptide sample can be fractionated using well known methods into partially or substantially purified protein fractions.

As used herein, the term “biological molecule” refers to any molecule found within a cell or produced by a living organism, including viruses. This term may include, but is not limited to, nucleic acids, polypeptides, carbohydrates, and lipids. A biological molecule can be isolated from various samples such as tissues of all kinds, cultured cells, body fluids, whole blood, blood serum, plasma, urine, feces, microorganisms, viruses, plants, and mixtures comprising nucleic acids following enzyme reactions. Examples of tissues include tissue from invertebrates, such as insects and mollusks, vertebrates such as fish, amphibians, reptiles, birds, and mammals such as humans, rats, dogs, cats and mice. Cultured cells can be from procaryotes, such as bacteria, blue green algae, actinomycetes, and mycoplasma and from eucaryotes, such as plants, animals, fungi, and protozoa. Blood samples include blood taken directly from an organism or blood that has been filtered in some way to remove some elements such as red blood cells, and/or serum or plasma. Nucleic acid can be isolated from enzyme reactions to purify the nucleic acid from enzymes such as DNA polymerase, RNA polymerase, reverse transcriptase, ligases, restriction enzymes, DNase, RNase, nucleases, proteases, and the like, or any other enzyme that can contact nucleic acids in a molecular biology method. Genomic DNA can be considered to be a “large biological molecule”.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments ±50%, in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The synthetic descriptions and specific examples that follow are only intended for the purposes of illustration, and are not to be construed as limiting in any manner to make compounds of the disclosure by other methods.

### Example 1

### Rapid Analysis of N-Glycoproteome of Human Serum and Peptide Isolation by Conjugation to Amino-Linking Beads

### Materials and Methods

Materials.

Hydrazide resin and sodium periodate were from Bio-Rad (Hercules, Calif.). BCA protein assay kit, Zeba spin desalting column (7k MWCO), Urea, and tris(2-carboxyethyl) phosphine (TCEP) were from Thermo Fisher Scientific (Waltham, Mass.). Sequencing-grade trypsin was from Promega (Madison, Wis.). PNGase F was from New England Biolabs (Ipswich, Mass.). alpha-CHC matrix was from Agilent Technology (Santa Clara, Calif.). Frits were from POREX (Fairburn, Ga.). All other chemicals were from Sigma-Aldrich (St. Louis, Mo.).

Preparation of Hydrazide Pipette Tip.

A round frit (2-mm-diameter and 1-mm-thick, pore size 15-45 microns) was first pushed into the pipette tip end (Disposable Automation Research Tips, Thermo Fisher Scientific, Waltham, Mass.). Two hundred microliters of hydrazide resin (50% slurry) was then loaded into each pipette tip. Liquids were blown out of the tip and a 5-mm round frit was pushed into the tip to secure the hydrazide resin between the two frits. The tips were then washed 5 times with 200 μL of water and conditioned 5 times with coupling buffer (100-mM sodium acetate, 1-M sodium chloride, pH 5.5) by aspirating and dispensing the solution. For less than 5% of the prepared tips, the flow was too slow due to high resistance, and the tips were therefore discarded.

Coupling Time for Glycoprotein to Hydrazide Tip.

Four hundred microliters of bovine fetuin in oxidation buffer (500 mM sodium acetate, 0.3 mM sodium chloride, pH 5) was oxidized with 15 mM sodium periodate for 1 h at room temperature in the dark followed by buffer exchange into coupling buffer. After addition of 100-mM aniline, the fetuin samples were slowly pipetted through hydrazide tips for coupling. Aliquots of fetuin samples were saved before, as well as after, fetuin was coupled for 1, 5, 10, 20, 30, 60 and 120 min. Protein concentration was determined using the BCA protein assay per manufacturer's protocol after removal of aniline. The absorbance was read at 562 nm with a spectrophotometer (BioTek, Winooski, Vt.). The results were plotted against time and data presented represent mean±SD (n=3).

Incubation Time for Trypsin Digestion. Bovine fetuin coupled to the hydrazide tips through oxidized glycans was washed with 3-mL urea buffer (8-M urea in 0.4-M NH₄HCO₃), reduced with 10-mM TCEP for 30 min, and alkylated with 12-mM iodoacetamide (IAA) for 15 min in the dark at room temperature (RT). After washing again with 3-mL urea buffer, the conjugated fetuin was digested with trypsin (1:30) in 100-mM ammonium bicarbonate where the digested non-glycopeptides were released into trypsin solution. Aliquots of trypsin solutions were saved before and after the samples were digested for 1, 5, 10, 20, 30, 60 and 120 min. The peptide concentration in each aliquot was then determined by BCA protein assay. The results were plotted against time and data presented represent mean±SD (n=3).

Incubation Time for PNGase F Release.

After digestion, the hydrazide tips (with conjugated glycopeptides) were washed extensively with 6-mL solutions of 1.5-M sodium chloride, 80% acetonitrile (ACN), deionized (DI) water, and 25-mM ammonium bicarbonate buffer to remove any residual non-glycopeptides released by digestion. 1500 U of PNGase F in 200 μL of 25-mM ammonium bicarbonate was then pipetted through the hydrazide tips. Aliquots of PNGase F solutions (with released peptides) were saved before and after releasing of any residual non-glycopeptides for 1, 5, 10, 20, 30, 60 and 120 min. A 10⁻¹² M angiotensin I standard in 50% ACN/1% TFA was used to serve as an internal standard. An equal amount of angiotensin I standard and samples (three sets of fetuin glycopeptides collected at various times of PNGase F incubations) were applied to matrix-assisted laser desorption/ionization (MALDI) spots, coated with alpha-CHC matrix and analyzed by matrix-assisted laser desorption/ionization time-of-flight/time-of-flight (MALDI-TOF/TOF) (4800, AB SCIEX, Framingham, Mass.). A total of 20 subspectra (100 shots/subspectrum) were averaged to yield the mass spectrum for each sample. Area under the curve for angiotensin I and the major fetuin glycopeptide released (LCPDCPLLAPLNDSR; SEQ ID NO:1) were recorded. The ratio of fetuin/angiotensin was calculated and plotted against time. Data presented represent mean±SD (n=3).

Isolation of N-linked Glycopeptides from Human Serum with a Hydrazide Tip.

N-linked glycopeptides were isolated from human serum using a hydrazide tip similar to that described above. Briefly, 40 μL of human serum (n=3) was diluted 1:1 with oxidation buffer, oxidized with sodium periodate, and buffer exchanged into coupling buffer. The serum sample was then slowly aspirated into hydrazide tips and dispensed back into a 96-well plate for 30 min using a liquid handling robotic system (Versette, Thermo Fisher Scientific, Waltham, Mass.). The aspiration and dispensing were repeated during the entire incubation time. The glycoproteins captured in the hydrazide tips were then reduced, alkylated, and digested for 1 h by pipetting the tips through TCEP, IAA and trypsin solutions (1:120 based on initial protein amount). The tips were then washed extensively and glycopeptides were released with 1500 U PNGase F in 25-mM ammonium bicarbonate buffer for 1 h at RT. Tips were then washed three times with 50% ACN and the eluents were combined and vacuumed to dryness. Samples were resuspended with 40 μl 5% ACN/0.2% formic acid. Two microliters of each sample were injected into a Q-Exactive mass spectrometer (Q-E, Thermo Fisher Scientific, Waltham, Mass.) for liquid chromatography-tandem mass spectrometry (LC-MS/MS) analysis.

LC-MS/MS Analysis.

Formerly N-linked glycopeptides were analyzed using a Q-E mass spectrometer with an EASY-Spray source. Peptides were separated with a 15-cm×75-μm C18 column on an Ultimate 3000 series UHPLC at a flow rate of 300 nL/min with a 110 min linear gradient (from 5 to 35% B over 75 min; A=0.1% formic acid 2% ACN in water, B=0.1% formic acid in 90% ACN). Full mass spectrometry (MS) scans were acquired over the mass range 400-1800 m/z with a mass resolution of 70,000. The AGC target value was set at 3,000,000. The fifteen most intense peaks were fragmented with Higher-energy Collisional Dissociation (HCD) with collision energy of 27. MS/MS was acquired with a resolution of 17,500 with an AGC target of 50,000 and max injection time of 200 ms. Dynamic exclusion was set for 15 sec.

Identification of Glycosites and Glycopeptide Quantification.

The resulting MS/MS spectra were searched against the European Bioinformatics Institute (http://www.ebi.ac.uk/) non-redundant International Protein Index human sequence database (IPI, v3.87, 2011/09/27, 91,491 entries) using Proteome Discoverer (v 1.4, Thermo Fisher Scientific, Waltham, Mass.). Base peak profiles of the three LC-MS/MS replicates or the three isolation replicates were opened and overlaid using the Xcalibur software (Thermo Fisher Scientific, Waltham, Mass.). For peptide identification, a mass tolerance of 10 ppm was permitted for intact peptide masses and 0.6 Da for HCD-fragmented ions, with allowance for two missed cleavages in the trypsin digests, oxidized methionine, and deamidated asparagine as potential variable modifications. Carboxyamidomethylation (C) was set as a fixed modification. Peptides with 1% FDR were reported with their peptide spectrum match (PSM). Peptides with N-glycosites (NXS/T, where X can be any amino acid except P) were required. For N-linked glycopeptides commonly identified in all three LC-MS/MS replicates or in all three isolation replicates, coefficient of variation (CV) for each peptide was calculated based on PSM; total PSMs was also calculated for each peptide by adding up the PSMs recorded in each run. The average CV for peptides with total PSMs over 150, between 150 and 60, between 60 and 30, between 30 and 15 and below 15 were calculated.

### Workflow Using the Presently Disclosed Hydrazide Tip

To achieve high throughput N-linked glycopeptide enrichment from serum, the presently disclosed subject matter provides a hydrazide tip for fast and reproducible N-linked glycopeptide isolation through solid phase extraction. FIG. 1 shows the flowchart of N-linked glycopeptide isolation with hydrazide tips above eppendorf tubes. Briefly, serum comprising proteins with oxidized glycans were pipetted through hydrazide tips in the presence of 100 mM aniline. Glycoproteins in the serum were conjugated covalently to the hydrazide resin packed in the tips. Glycoproteins captured on the tips were then denatured, reduced, alkylated, and digested by aspirating and dispensing the hydrazide tips in urea, TCEP, IAA and trypsin solution, respectively. The tips were then washed extensively with 1.5-M sodium chloride, 80% ACN, deionized (DI) water, and 25-mM ammonium bicarbonate buffer to removed residual non-glycopeptides. Finally, the formerly N-linked glycopeptides were released by pipetting the hydrazide tips in PNGase solution.

### Incubation Times for the Major Steps of the Presently Disclosed Methods Using a Hydrazide Tip

To determine the reaction times of the major steps of the presently disclosed methods, i.e. coupling, proteolysis and PNGase F release for glycopeptide capture of serum, bovine fetuin, a 38 kD glycoprotein with three N-linked glycosylation sites, was used as a standard.

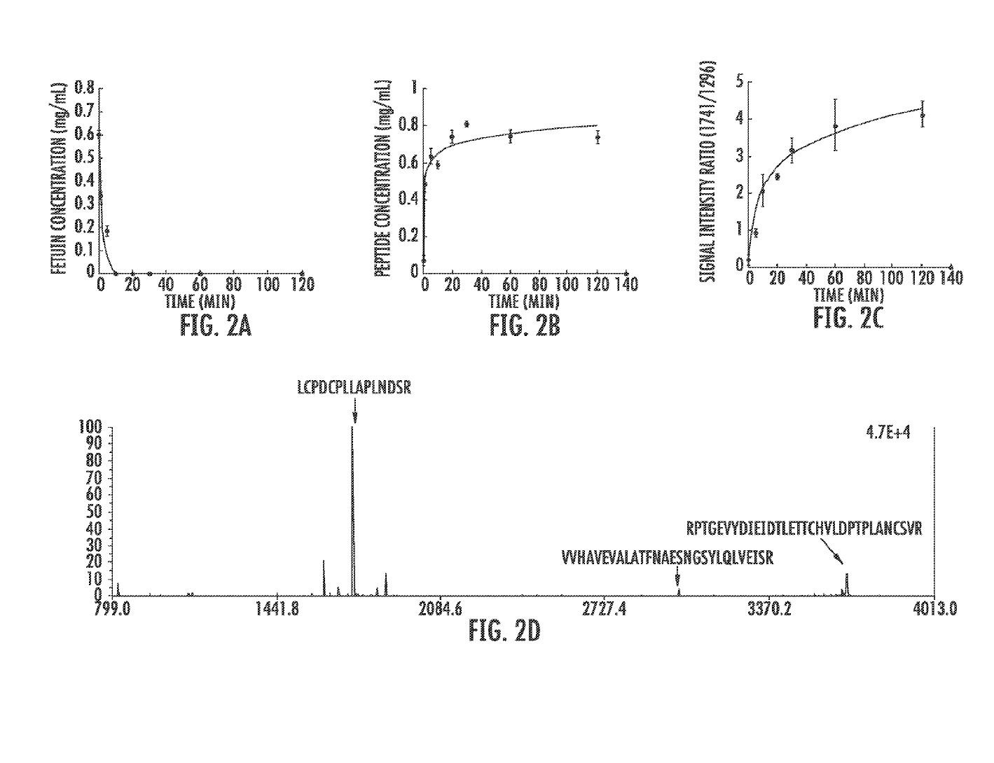

To determine the incubation time required for complete coupling of the glycoproteins to the hydrazide tip, 0.8 mg oxidized bovine fetuin proteins were coupled with hydrazide tips in the presence of 100-mM aniline for various times. The amount of fetuin used here equals the amount of glycoprotein estimated from 40 μL of human serum. Aniline was used as a catalyst to improve the reaction rate between aldehyde and hydrazide groups as previously reported (Zeng et al., 2009; Dirksen et al., 2010). It was found that essentially no fetuin was present in the solution at 10 min, suggesting that coupling was complete after a 10 min incubation (FIG. 2A).

To determine the incubation time required for trypsin digestion, the fetuin proteins coupled to the hydrazide tips above were denatured, reduced, and alkylated. The fetuin samples were then digested with trypsin using a trypsin-to-glycoprotein ratio of 1:30 for various times. This trypsin-to-glycoprotein ratio was also used in the serum glycopeptide isolation where glycoproteins account for about 25% of the total serum proteins. It was found that no additional peptides were released into the trypsin solutions after 1 h, suggesting that trypsin digestion was complete at 1 h (FIG. 2B).

To determine the incubation time required for PNGase F release of formerly N-linked glycopeptides, the hydrazide tips were washed extensively with 1.5-M sodium chloride, 80% ACN, DI water, and 25-mM ammonium bicarbonate buffer to remove any residual non-glycopeptides released by digestion. PNGase F in 25-mM ammonium bicarbonate was then pipetted through the hydrazide tips for various times. Again, the PNGase F-to-glycoprotein ratio is similar to that used in serum glycopeptide isolations. As shown in FIG. 2C, most peptides were released after 1 h. At this time point, all three predicted formerly N-linked glycopeptides of fetuin (LCPDCPLLAPLNDSR, SEQ ID NO:1; VVHAVEVALATFNAESNGSYLQLVEISR, SEQ ID NO:2; and RPTGEVYDIEIDTLETTCHVLDPTPLANCSVR, SEQ ID NO:3) could be observed by MALDI-TOF-TOF (FIG. 2D).

Thus, with the presently disclosed hydrazide tip and methods thereof, the total time required to complete N-linked glycopeptide isolation was within 8 h. The hydrazide tip contains hydrazide resins 40-60 micrometers in size with 0.1-μm micropores. After packing, the spacing between resins is estimated to be roughly 50-90 micrometers considering a face-centered cubic or hexagonal close-packed arrangement (Conway et al., 1999). Without wishing to be bound to any one particular theory, it is believed that such small dimensions enable the presently disclosed hydrazide tips to work as a microfluidic reactor, where the reaction rate is significantly improved due to faster mixing (Sia and Whitesides, 2003). As shown above, the presently disclosed methods decreased the processing time to less than 8 hours. In addition, the isolation capacity could be easily adjusted by simply controlling the amount of hydrazide beads packed into each tip. As the loading capacity of the hydrazide beads is about 40-μL serum/200-μL hydrazide beads (50% slurry) as previously reported (Zhou et al., 2007), the hydrazide beads packed could be adjusted accordingly for optimal performance when a different amount of serum needs to be processed. Moreover, the presently disclosed workflow methods provided herein could be used to isolate N-linked glycopeptides in diverse types of samples, such as body fluids. Finally, as the presently disclosed hydrazide tip could be readily used in liquid handling robotic systems, in some embodiments, the presently disclosed methods provide automation of N-linked glycopeptide isolation for high throughput sample preparation.

### Rapid Analysis of N-Glycoproteome of Human Serum

To attempt automation of isolation of N-linked glycopeptides, the hydrazide tips were used in combination with a liquid handling robotic system to perform glycopeptide isolation from human serum. Forty microliters of serum was processed with each hydrazide tip and 1/20th glycopeptide isolated was injected into a Q-E mass spectrometer for LC-MS/MS analysis.

Table 1 shows the identification, specificity and missed cleavage of glycopeptides isolated using hydrazide tip and the original SPEG procedure. Formerly N-linked glycopeptides were isolated from 40 μL of human serum with the presently disclosed methods using a hydrazide tip or with the original SPEG method. 1/20th of the glycopeptides isolated was injected into a QE mass spectrometer for LC-MS/MS analysis. The number and specificity of formerly N-linked glycopeptides identified as well as the percentage of peptides with missed cleavage were listed for each isolation.

After controlling the FDR<1% for peptide identification, 332, 345 and 328 unique formerly N-linked glycopeptides from human serum were identified in Isolations 1, 2, and 3, respectively (Table 1). In comparison, a similar number of unique glycopeptides, 315, was identified from the same human serum when the isolation was carried out using the original SPEG isolation method (Zhang et al., 2003). The specificity of N-linked glycopeptides identified was also similar between the hydrazide tip isolations (89.04%, 86.59% and 90.07%) and the original SPEG isolation method (81.66%). The missed cleavages observed were 20.22%, 21.07% and 20.30%, for Isolations 1, 2, and 3, respectively, and 16.38% for the original SPEG isolation method.

Table 2 shows the unique formerly N-linked glycopeptides of human serum identified in three isolation replicates. Human serum samples were subjected to N-linked glycopeptide isolation with the presently disclosed hydrazide tips. An aliquot of the formerly N-linked glycopeptides from each isolation (n=3) was injected once into a Q-E mass spectrometer for LC-MS/MS analysis. The sequences of the unique peptides identified are listed with their peptide spectral match (PSM).

Table 3 shows the unique formerly N-linked glycopeptides of human serum identified in three LC-MS/MS Replicates. Human serum samples were subjected to N-linked glycopeptide isolation with the presently disclosed hydrazide tips. An aliquot of the formerly N-linked glycopeptides was injected three times into a Q-E mass spectrometer for LC-MS/MS analysis. The sequences of the unique peptides identified are listed with their peptide spectral match (PSM).

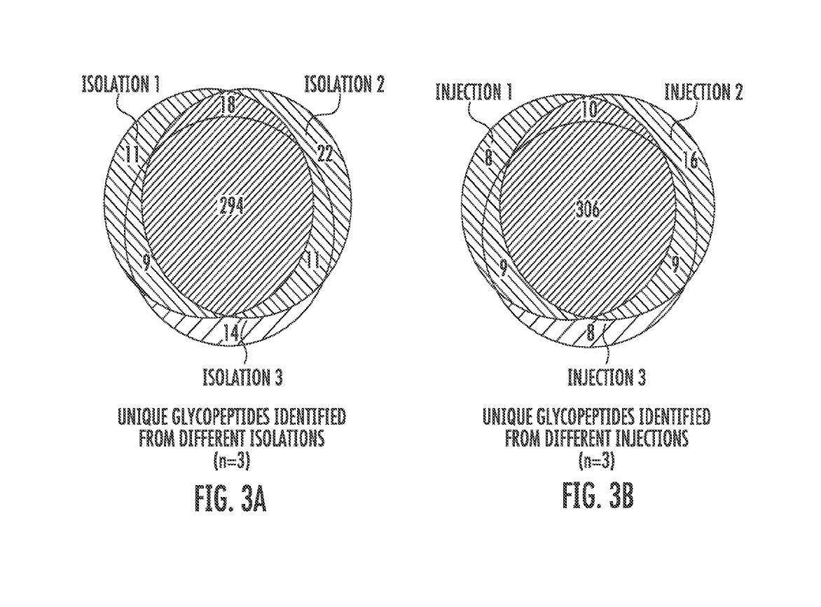

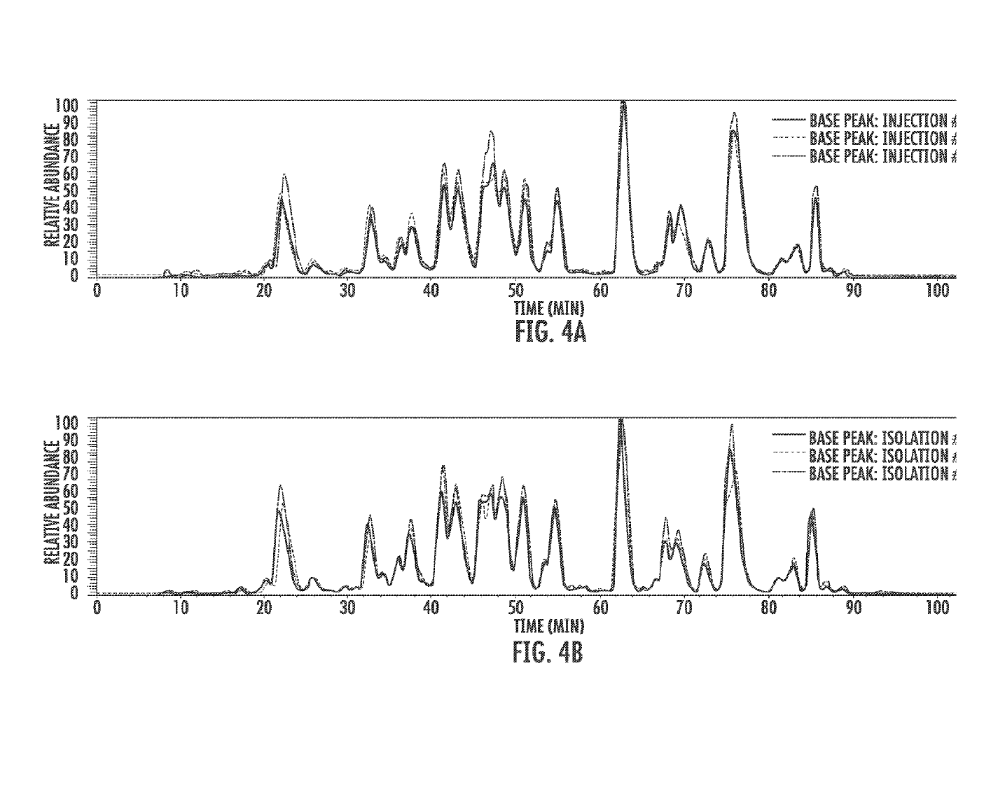

Altogether, a total of 379 unique formerly N-linked glycopeptides were identified in the three isolation replicates with 294 commonly identified (FIG. 3A; Table 2). Similarly, a total of 366 unique formerly N-linked glycopeptides were identified in the three LC-MS/MS replicates, with 306 of them commonly identified (FIG. 3B; Table 3). In both cases, the commonly identified peptides were about 80% of that totally identified. In addition, great consistency was observed in the LC profiles between the LC-MS/MS replicates and the isolation replicates (FIG. 4).

Table 4 shows the reproducibility of glycopeptide isolations using the presently disclosed hydrazide tip. Formerly N-linked glycopeptides from 40 μl human serum were isolated three times in parallel with the presently disclosed methods and hydrazide tip. 1/20th of the glycopeptides isolated from Isolation 1 was injected three times into a QE mass spectrometer for LC-MS/MS analysis; 1/20th of the glycopeptides isolated from Isolations 2 and 3 was injected once into a QE mass spectrometer for LC-MS/MS analysis. The MS/MS spectra generated were searched against human IPI 3.87 for identification of glycopeptides. Peptide spectral matches (PSMs) reported for each glycopeptide were used to calculate the coefficient of variations (CVs) between injections and between isolations. The CVs were listed along with the total number of PSMs added up from each run.

Table 4 shows that the reproducibility between isolation replicates was comparable to that between LC-MS/MS replicates, with CVs, based on the PSMs, only slightly higher between isolations (Table 4). Overall, the CVs increased as the PSM of glycopeptides decreased as reported before (Liu et al., 2004). The CVs between isolations were 6.32%, 11.36%, 9.98%, 17.01% and 28.1% for glycopeptides with a total PSM over or equal to 150, between less than 150 and more than or equal to 60, between less than 60 and more than or equal to 30, between less than 30 and more than or equal to 15, and less than 15, respectively. In comparison, the CVs between LC-MS/MS replicates were 4.53%, 6.27%, 8.57%, 11.53% and 21.55% for glycopeptides with a total PSM over or equal to 150, between less than 150 and more than or equal to 60, between less than 60 and more than or equal to 30, between less than 30 and more than or equal to 15, and less than 15. These data demonstrate that glycopeptide isolation with hydrazide tips has high throughput, great reproducibility, and automation capability when used in combination with liquid handling robotic systems.

TABLE 1
Identification, Specificity and Missed Cleavage of Glycopeptides
Isolated Using Hydrazide Tip and the Original SPEG Procedure

| | Glycopeptides | | Missed |
|---|---|---|---|
| Sample | Identified | Specificity | Cleavage |
| Hydrazide Tip Isolation 1 | 332 | 89.04% | 20.22% |
| Hydrazide Tip Isolation 2 | 345 | 86.59% | 21.07% |
| Hydrazide Tip Isolation 3 | 328 | 90.07% | 20.30% |
| Original SPEG Isolation | 315 | 81.66% | 16.38% |

TABLE 2
Unique Formerly N-linked Glycopeptides of Human Serum Identified in
Three Isolation Replicates.

| | SEQ | Peptide Spectrum Match | | |
|---|---|---|---|---|
| | ID | Isolation | Isolation | Isolation |
| Sequence | NO: | 1 | 2 | 3 |
| AALAAFnAQNnGSnFQLEEISR | 4 | 119 | 121 | 105 |
| AATcINPLnGSVcERPAnHSAK | 5 | 1 | 3 | 2 |
| ADGTVnQIEGEATPVnLTEPAK | 6 | 19 | 21 | 21 |
| ADGTVNQIEGEATPVnLTEPAKLEVK | 7 | 13 | 14 | 11 |
| ADTHDEILEGLNFnLTEIPEAQIH | 8 | 8 | 6 | 6 |
| ADTHDEILEGLnFnLTEIPEAQIHEGFQELLR | 9 | 117 | 118 | 119 |
| AELSnHTRPVILVPGcLGNQLEAK | 10 | 2 | 3 | 2 |
| AFEnVTDLQWLILDHnLLEnSK | 11 | 18 | 15 | 11 |
| AFHYnVSSHGcQLLPWTQHSPHTR | 12 | 2 | 1 | #N/A |
| AFITnFSMIIDGmTYPGIIK | 13 | 7 | 5 | 5 |
| AFITnFSMIIDGmTYPGIIKEK | 14 | 3 | 3 | 2 |
| AGAFLGLTNVAVmnLSGNcLR | 15 | 8 | 9 | 6 |
| AGLQAFFQVQEcnK | 16 | 7 | 7 | 6 |
| AHLnVSGIPcSVLLADVEDLIQQQISnDTVSPR | 17 | 1 | 2 | 1 |
| ALPQPQnVTSLLGcTH | 18 | 5 | 7 | 16 |
| ALQAVYSmmSWPDDVPPEGWnR | 19 | 1 | 3 | #N/A |
| AMMAFTADLFSLVAQTSTcPNLILSPLSVALALSHLALGAQnHTLQR | 20 | 1 | #N/A | #N/A |
| AnLSSQALQmSLDYGFVTPLTSmSIR | 21 | 13 | 12 | 11 |
| APDKNVIFSPLSISTALAFLSLGAHnTTLTEILK | 22 | 9 | 9 | 10 |
| AQLLQGLGFnLTER | 23 | 18 | 17 | 20 |
| AQVIInITDVDEPPIFQQPFYHFQLK | 24 | 2 | 4 | 3 |
| AREDIFMETLKDIVEYYnDSnGSHVLQGR | 25 | 21 | 22 | 24 |
| AVLQLnEEGVDTAGSTGVTLnLTSKPIILR | 26 | 37 | 34 | 34 |
| AVnITSENLIDDVVSLIR | 27 | 10 | 11 | 8 |
| AYLLPAPPAPGnASESEEDR | 28 | 3 | 4 | 3 |
| cATPHGDnASLEATFVK | 29 | 3 | 4 | 3 |
| cGLVPVLAENYnK | 30 | 4 | 6 | 7 |
| cGncSLTTLKDEDFcK | 31 | 7 | 7 | 6 |
| cGncSLTTLKDEDFcKR | 32 | 7 | 8 | 7 |
| cIQAnYSLmENGK | 33 | 21 | 21 | 21 |
| cIQAnYSLmEnGKIK | 34 | 2 | 1 | 3 |
| cmWSSALnSLnLSFAGLEQVPK | 35 | 3 | 4 | 1 |
| cSDGWSFDATTLDDnGTmLFFK | 36 | 19 | 26 | 16 |
| DFVnASSKYEITTIHnLFR | 37 | 3 | 6 | 4 |
| DHEnGTGTNTYAALNSVYLmmNNQmR | 38 | 6 | 10 | 7 |
| DIVEYYNDSnGSHVLQGR | 39 | 37 | 36 | 30 |
| DKIcDLLVANNHFAHFFAPQnLTNmNK | 40 | 31 | 31 | 35 |
| DmTEVISSLENAnYKDHENGTGTNTYAALNSVYLMMNNQMR | 41 | 6 | 8 | 8 |
| DQcIVDDITYNVnDTFHK | 42 | 11 | 11 | 11 |
| DQcIVDDITYNVnDTFHKR | 43 | 2 | 1 | 1 |
| DRQDGEEVLQcmPVcGRPVTPIAQnQTTLGSSR | 44 | 1 | 1 | 1 |
| DSVSVVLGQHFFnR | 45 | 3 | 3 | 5 |
| DTAVFEcLPQHAmFGNDTITcTTHGnWTK | 46 | 32 | 34 | 25 |
| DTAVFEcLPQHAmFGnDTITcTTHGnWTKLPEcR | 47 | 10 | 7 | 10 |
| DVQIIVFPEDGIHGFnFTR | 48 | 7 | 8 | 8 |
| EDIFmETLKDIVEYYnDSNGSHVLQGR | 49 | 4 | 4 | 4 |
| EEQYNSTYRVVSVLTVLHQDWLnGK | 50 | 1 | #N/A | 1 |
| EEQYnSTYRVVSVLTVLHQDWLnGKEYK | 51 | 1 | #N/A | 1 |
| EGDHEFLEVPEAQEDVEATFPVHQPGnYScSYR | 52 | 17 | 17 | 19 |
| EGYSnISYIVVNHQGISSR | 53 | 9 | 10 | 10 |
| EHEAQSnASLDVFLGHTNVEELmK | 54 | 9 | 10 | 5 |
| EHEGAIYPDnTTDFQR | 55 | 26 | 24 | 27 |
| EHETcLAPELYNGnYSTTQK | 56 | 5 | 5 | 5 |
| EHYnLSAATcSPGQmcGHYTQVVWAK | 57 | 2 | 2 | 2 |
| ELDREVYPWYnLTVEAK | 58 | 1 | 1 | #N/A |
| ELHHLQEQnVSNAFLDK | 59 | 27 | 25 | 27 |
| ELHHLQEQnVSnAFLDKGEFYIGSK | 60 | 94 | 74 | 79 |
| ELPGVcnETmmALWEEcKPcLK | 61 | 10 | 14 | 8 |
| EnLTAPGSDSAVFFEQGTTR | 62 | 16 | 15 | 16 |
| ERSWPAVGncSSALR | 63 | 2 | 2 | 1 |
| EVnTSGFAPARPPPQPGSTTFWAWSVLR | 64 | 5 | 5 | 4 |
| EVSFLncSLDnGGcTHYcLEEVGWR | 65 | 5 | 6 | 7 |
| EVYPWYnLTVEAK | 66 | 2 | 3 | 1 |
| EWEKELHHLQEQnVSnAFLDKGEFYIGSK | 67 | 5 | 2 | 3 |
| EYESYSDFERnVTEK | 68 | 1 | 1 | 2 |
| FcRDnYTDLVAIQNK | 69 | 2 | #N/A | 2 |
| FDFQGTcEYLLSAPcHGPPLGAEnFTVTVAnEHR | 70 | 1 | #N/A | 1 |
| FEDGVLDPDYPRnISDGFDGIPDNVDAALALPAHSYSGR | 71 | 7 | 10 | 9 |
| FEVDSPVYnATWSASLK | 72 | 3 | 4 | 4 |
| FGHSAVLHnSTMYVFGGFNSLLLSDILVFTSEQcDAHR | 73 | 5 | 8 | 6 |
| FHDVSESTHWTPFLnASVHYIR | 74 | 6 | 6 | 6 |
| FLNnGTcTAEGK | 75 | 3 | 1 | 3 |
| FLTEVEKnATALYHVEAFK | 76 | 1 | #N/A | #N/A |
| FnFQGTcEYLLSAPcHGPPLGAEnFTVTVAnEHR | 77 | 1 | 2 | 1 |
| FnLTETSEAEIHQSFQHLLR | 78 | 182 | 173 | 175 |
| FNPGAESVVLSnSTLK | 79 | 1 | 1 | 2 |
| FnSSYLQGTNQITGR | 80 | 2 | 1 | 1 |
| FQSPAGTEALFELHNISVADSAnYScVYVDLKPPFGGSAPSER | 81 | 93 | 79 | 82 |
| FSDGLESnSSTQFEVK | 82 | 52 | 63 | 59 |
| FSDGLESnSSTQFEVKK | 83 | 3 | 3 | 3 |
| FSLLGHASIScTVEnETIGVWRPSPPTcEK | 84 | 73 | 71 | 65 |
| FSYSKnETYQLFLSYSSK | 85 | 10 | 10 | 10 |
| FVGTPEVnQTTLYQR | 86 | 1 | 2 | 1 |
| FVQAIcEGDDcQPPAYTYNnITcASPPEVVGLDLR | 87 | 6 | 3 | 2 |
| FVQGnSTEVAcHPGYGLPK | 88 | 4 | 3 | 3 |
| GAFISnFSMTVDGK | 89 | 9 | 9 | 16 |
| GcnDSDVLAVAGFALR | 90 | 3 | 3 | 4 |
| GcScFSDWQGPGcSVPVPAnQSFWTR | 91 | 4 | 6 | 2 |
| GcVLLSYLnETVTVSASLESVR | 92 | 132 | 129 | 129 |
| GDSGGPLVcmDANnVTYVWGVVSWGEncGKPEFPGVYTK | 93 | 12 | 12 | 13 |
| GETHEQVHSILHFKDFVnASSK | 94 | 1 | 2 | 1 |
| GETHEQVHSILHFKDFVnASSKYEITTIHNLFR | 95 | 1 | 1 | #N/A |
| GFGVAIVGnYTAALPTEAALR | 96 | 49 | 54 | 48 |
| GFLALYQTVAVnYSQPISEASR | 97 | 7 | 7 | 6 |
| GGETAQSADPQWEQLNNKnLSmPLLPADFHK | 98 | 13 | 15 | 11 |
| GGnSnGALcHFPFLYNNHnYTDcTSEGR | 99 | 14 | 14 | 15 |
| GGNSNGALcHFPFLYNnHnYTDcTSEGRR | 100 | 1 | #N/A | #N/A |
| GHFIYKnVSEDLPLPTFSPTLLGDSR | 101 | 1 | 3 | #N/A |
| GLKFnLTETSEAEIHQSFQHLLR | 102 | 86 | 90 | 71 |
| GLnLTEDTYKPR | 103 | 1 | #N/A | 1 |
| GLTFQQnASSmcGPDQDTAIR | 104 | 8 | 10 | 11 |
| GLTFQQnASSmcVPDQDTAIR | 105 | 9 | 10 | 11 |
| GmnLTVFGGTVTAFLGIPYAQPPLGR | 106 | 4 | 1 | 2 |
| GNEANYYSnATTDEHGLVQFSInTTnVmGTSLTVR | 107 | 134 | 124 | 128 |
| GNVAVTVSGHTcQHWSAQTPHTHnR | 108 | 9 | 11 | 8 |
| GPSTPLPEDPnWnVTEFHTTPK | 109 | 1 | #N/A | #N/A |
| GTAnTTTAGVPcQR | 110 | 2 | 2 | #N/A |
| GTGnDTVLNVALLNVISNQEcNIK | 111 | 1 | 1 | #N/A |
| GVTSVSQIFHSPDLAIRDTFVnASR | 112 | 3 | 4 | 5 |
| HAnWTLTPLK | 113 | 5 | 7 | 6 |
| HEEGHmLncTcFGQGR | 114 | 6 | 4 | 6 |
| HGIQYFnnNTQHSSLFmLnEVK | 115 | 29 | 41 | 27 |
| HGIQYFNnNTQHSSLFmLnEVKR | 116 | 14 | 13 | 15 |
| HGIQYFnnNTQHSSLFTLnEVK | 117 | 40 | 41 | 40 |
| HGIQYFnnNTQHSSLFTLNEVKR | 118 | 11 | 10 | 10 |
| HGVIISSTVDTYEnGSSVEYR | 119 | 10 | 11 | 10 |
| HLQmDIHIFEPQGISFLETESTFmTNQLVDALTTWQnK | 120 | 5 | 9 | 6 |
| HYLVSnISHDTVLQcHFTcSGK | 121 | 3 | 2 | 3 |
| HYTnSSQDVTVPcR | 122 | 13 | 13 | 13 |
| HYYIAAEEIIWnYAPSGIDIFTKEnLTAPGSDSAVFFEQGTTR | 123 | 11 | 10 | 10 |
| IADAHLDRVEnTTVYYLVLDVQESDcSVLSR | 124 | 68 | 58 | 64 |
| IcDLLVAnNHFAHFFAPQnLTNmnK | 125 | 23 | 21 | 23 |
| IDSTGnVTNELR | 126 | 3 | 5 | 3 |
| IITILEEEmnVSVcGLYTYGKPVPGHVTVSIcR | 127 | 74 | 67 | 64 |
| INNDFNYEFYnSTWSYVK | 128 | 6 | 6 | 6 |
| IPcSQPPQIEHGTInSSR | 129 | 21 | 19 | 19 |
| ISEEnETTcYMGK | 130 | 14 | 16 | 14 |
| ISnSSDTVEcEcSENWK | 131 | 5 | 4 | 3 |
| ISNSSDTVEcEcSEnWKGEAcDIPHcTDNcGFPHR | 132 | 3 | 5 | 6 |
| ITPnLAEFAFSLYRQLAHQSnSTnIFFSPVSIATAFAmLSLGTK | 133 | 2 | #N/A | #N/A |
| ITYSIVQTncSK | 134 | 12 | 12 | 14 |
| ITYSIVQTncSKEnFLFLTPDcK | 135 | 11 | 13 | 11 |
| IVGGTnSSWGEWPWQVSLQVK | 136 | 8 | 9 | 6 |
| IVLDPSGSMnlYLVLDGSDSIGASnFTGAK | 137 | 134 | 130 | 132 |
| IYPGVDFGGEELnVTFVK | 138 | 7 | 5 | 7 |
| IYSGILnLSDITK | 139 | 7 | 7 | 8 |
| IYSnHSALESLALIPLQAPLK | 140 | 3 | 4 | 4 |
| KAFITnFSMIIDGmTYPGIIK | 141 | 4 | 5 | 4 |
| KAFITnFSMIIDGmTYPGIIKEK | 142 | 4 | 7 | 3 |
| KcGncSLTTLKDEDFcK | 143 | 1 | 1 | 2 |
| KDFEDLYTPVnGSIVIVR | 144 | 2 | 2 | #N/A |
| KEHETcLAPELYNGnYSTTQK | 145 | 12 | 11 | 12 |
| KIVLDPSGSmnIYLVLDGSDSIGASnFTGAK | 146 | 42 | 35 | 37 |
| KLHINHNnLTESVGPLPK | 147 | 6 | 7 | 6 |
| KLINDYVKnGTR | 148 | 2 | 2 | 2 |
| KLPPGLLAnFTLLR | 149 | 5 | 6 | 6 |
| KnQSVNVFLGHTAIDEmLK | 150 | 3 | 5 | 4 |
| KQVHFFVnASDVDNVK | 151 | 5 | 5 | 4 |
| KVcQDcPLLAPLnDTR | 152 | 12 | 15 | 16 |
| LAGKPTHVnVSVVMAEVDGTcY | 153 | 64 | 59 | 66 |
| LAnLTQGEDQYYLR | 154 | 15 | 15 | 15 |
| LATALSLSNKFVEGSHnSTVSLTTK | 155 | 2 | 4 | 2 |
| LDAPTNLQFVnETDSTVLVR | 156 | 9 | 6 | 6 |
| LDPVSLQTLQTWnTSYPK | 157 | 1 | 2 | 1 |
| LDREnISEYHLTAVIVDK | 158 | 1 | 2 | 1 |
| LDREnISEYHLTAVIVDKDTGENLETPSSFTIK | 159 | 1 | 2 | 1 |
| LEDLEVTGSSFLnLSTnIFSnLTSLGK | 160 | 12 | 18 | 12 |
| LEPVHLQLQcMSQEQLAQVAAnATK | 161 | 12 | 12 | 12 |
| LETTVnYTDSQRPIcLPSK | 162 | 3 | 4 | 3 |
| LFGDKSLTFnETYQDISELVYGAK | 163 | 4 | 5 | 6 |
| LGAcnDTLQQLMEVFK | 164 | 34 | 34 | 35 |
| LGAcnDTLQQLmEVFKFDTISEK | 165 | 9 | 10 | 10 |
| LGAcnDTLQQLMEVFKFDTISEKTSDQIHFFFAK | 166 | 3 | 4 | 3 |
| LGHcPDPVLVnGEFSSSGPVnVSDK | 167 | 9 | 10 | 8 |
| LGSFEGLVnLTFIHLQHNR | 168 | 13 | 11 | 12 |
| LGSLQELFLDSNnISELPPQVFSQLFcLER | 169 | 3 | 4 | 4 |
| LGSYPVGGnVSFEcEDGFILR | 170 | 5 | 7 | 6 |
| LGTSLSSGHVLMnGTLK | 171 | 8 | 10 | 9 |
| LHINHNnLTESVGPLPK | 172 | 11 | 10 | 10 |
| LKELPGVcnETMmALWEEcKPcLK | 173 | 11 | 20 | 12 |
| LLLSQLDSHPSHSAVVnWTSYASSIEALSSGNK | 174 | 1 | 2 | 1 |
| LNAEnnATFYFK | 175 | 108 | 117 | 103 |
| LnDTLDYEcHDGYESnTGSTTGSIVcGYnGWSDLPIcYER | 176 | 14 | 13 | 18 |
| LNVEAAnWTVR | 177 | 4 | 3 | 3 |
| LPASLAEYTVTQLRPnATYSVcVmPLGPGR | 178 | 1 | #N/A | #N/A |
| LPPGLLAnFTLLR | 179 | 3 | 6 | 4 |
| LPTQnITFQTESSVAEQEAEFQSPK | 180 | 30 | 34 | 33 |
| LPYQGnATmLVVLmEK | 181 | 2 | 1 | 1 |
| LQAILGVPWKDKncTSR | 182 | 13 | 13 | 11 |
| LQAPLnYTEFQKPIcLPSK | 183 | 7 | 8 | 7 |
| LQNnENnIScVER | 184 | 7 | 6 | 8 |
| LSDLSInSTEcLHVHcR | 185 | 84 | 72 | 78 |
| LSHnELADSGIPGNSFnVSSLVELDLSYNK | 186 | 26 | 20 | 28 |
| LSLHRPALEDLLLGSEAnLTcTLTGLR | 187 | 92 | 77 | 85 |
| LSSWVLLmKYLGnATAIFFLPDEGK | 188 | 1 | #N/A | 1 |
| LSVDKDQYVEPEnVTIQcDSGYGVVGPQSITcSGnR | 189 | 5 | 8 | 3 |
| LTDTIcGVGnmSAnASDQER | 190 | 5 | 7 | 4 |
| LVSANRLFGDKSLTFnETYQDISELVYGAK | 191 | 3 | 2 | 4 |
| LYHFLLGAWSLnATELDPcPLSPELLGLTK | 192 | 15 | 13 | 8 |
| LYLGSnnLTALHPALFQnLSK | 193 | 12 | 12 | 11 |
| mAGKPTHInVSVVmAEADGTcY | 194 | 2 | 3 | 4 |
| mAGKPTHVnVSVVmAEVDGTcY | 195 | 3 | 2 | 4 |
| mAWPEDHVFISTPSFnYTGR | 196 | 6 | 5 | 4 |
| MDGASnVTcINSR | 197 | 25 | 27 | 20 |
| MLLTFHTDFSNEEnGTImFYK | 198 | 1 | 3 | #N/A |
| mLNnNTGIYTcSAQGVWmNK | 199 | 1 | #N/A | #N/A |
| mLnTSSLLEQLnEQFNWVSR | 200 | 26 | 35 | 23 |
| mPSQAPTGNFYPQPLLnSSmcLEDSR | 201 | 4 | 7 | 4 |
| mQcLAAALKDETnMSGGGEQADILPAnYVVKDR | 202 | 1 | #N/A | #N/A |
| mSnITFLnFDPPIEEFHQYYQHIVTTLVK | 203 | 1 | 2 | #N/A |
| mVSHHnLTTGATLInEQWLLTTAK | 204 | 443 | 390 | 483 |
| mVSHHnLTTGATLINEQWLLTTAKNLFLnHSEnATAK | 205 | 10 | 11 | 11 |
| mVTAFTTccTLSEEFAcVDNLADLVFGELcGVNEnR | 206 | 2 | 4 | 3 |
| NAHGEEKEnLTAR | 207 | 1 | #N/A | #N/A |
| ncGVncSGDVFTALIGEIASPnYPKPYPEnSR | 208 | 8 | 9 | 9 |
| NcQDIDEcVTGIHncSInETcFNIQGGFR | 209 | 1 | 2 | 2 |
| NEEYnKSVQEIQATFFYFTPnKTEDTIFLR | 210 | 12 | 11 | 10 |
| nEMLEIQVFNYSKVFSnK | 211 | 2 | #N/A | #N/A |
| nGTGHGnSTHHGPEYmR | 212 | 2 | 5 | 6 |
| NHPnITFFVYVSnFTWPIK | 213 | 4 | 4 | 3 |
| nISDGFDGIPDNVDAALALPAHSYSGR | 214 | 2 | 2 | 1 |
| nLASRPYTFHSHGITYYKEHEGAIYPDnTTDFQR | 215 | 5 | 4 | 5 |
| NLFLnHSEnATAK | 216 | 243 | 258 | 279 |
| NLFLnHSEnATAKDIAPTLTLYVGK | 217 | 11 | 11 | 10 |
| NLFLnHSEnATAKDIAPTLTLYVGKK | 218 | 2 | 3 | 3 |
| nnATVHEQVGGPSLTSDLQAQSK | 219 | 51 | 46 | 31 |
| NnmSFVVLVPTHFEWnVSQVLAnLSWDTLHPPLVWERPTK | 220 | 2 | 3 | 1 |
| NPPmGGNVVIFDTVITNQEEPYQnHSGR | 221 | 5 | 6 | 9 |
| NPVGLIGAEnATGETDPSHSK | 222 | 11 | 11 | 11 |
| nQALnLSLAYSFVTPLTSmVVTKPDDQEQSQVAEKPmEGESR | 223 | 9 | 12 | 8 |
| NSVLnSSTAEHSSPYSEDPIEDPLQPDVTGIR | 224 | 3 | 4 | 4 |
| nVIFSPLSISTALAFLSLGAHnTTLTEILK | 225 | 11 | 14 | 11 |
| QDQcIYnTTYLNVQR | 226 | 181 | 176 | 169 |
| QDQcIYnTTYLNVQREnGTISR | 227 | 5 | 5 | 5 |
| QEDLSVGSVLLTVnATDPDSLQHQTIR | 228 | 1 | 1 | 1 |
| QGGVnATQVLIQHLR | 229 | 1 | 1 | 1 |
| QInSSISGNLWDKDQR | 230 | 3 | 3 | 2 |
| QLAHQSnSTnIFFSPVSIATAFAmLSLGTK | 231 | 92 | 95 | 80 |
| QLDMLDLSnNSLASVPEGLWASLGQPNWDMR | 232 | 16 | 14 | 9 |
| QLEEFLnQSSPFYFWmNGDR | 233 | 27 | 28 | 28 |
| QLEEFLnQSSPFYFWmnGDRIDSLLEnDR | 234 | 11 | 13 | 12 |
| QLVEIEKVVLHPnYSQVDIGLIK | 235 | 5 | 3 | 5 |
| QNESHnFSGDIALLELQHSIPLGPNVLPVcLPDnETLYR | 236 | 5 | 6 | 6 |
| QnQcFYnSSYLnVQR | 237 | 18 | 16 | 13 |
| QPQAGLSQAnFTLGPVSR | 238 | 1 | 1 | #N/A |
| QQQHLFGSnVTDcSGnFcLFR | 239 | 160 | 159 | 160 |
| QVHFFVnASDVDNVK | 240 | 12 | 12 | 11 |
| QVLFLDTVYGncSTHFTVK | 241 | 4 | 6 | 4 |
| QVQVLQnLTTTYEIVLWQPVTADLIVK | 242 | 2 | 2 | 3 |
| REGDHEFLEVPEAQEDVEATFPVHQPGnYScSYR | 243 | 12 | 12 | 14 |
| RHEEGHmLncTcFGQGR | 244 | 1 | #N/A | 1 |
| RNPPmGGNVVIFDTVITNQEEPYQnHSGR | 245 | 12 | 15 | 14 |
| SDHGSSIScQPPAEIPGYLPADTVHLAVEFFnLTHLPANLLQGASK | 246 | 10 | 11 | 9 |
| SHAASDAPEnLTLLAETADAR | 247 | 1 | 1 | 1 |
| SHEIWTHScPQSPGnGTDASH | 248 | 2 | 3 | 1 |
| SIPAcVPWSPYLFQPnDTcIVSGWGR | 249 | 13 | 13 | 13 |
| SKPTVSSSmEFKYDFnSSmLYSTAK | 250 | 1 | 2 | 1 |
| SKWnITmESYVVHTnYDEYAIFLTK | 251 | 19 | 19 | 15 |
| SLGnVnFTVSAEALESQELcGTEVPSVPEHGR | 252 | 157 | 136 | 146 |
| SLGnVnFTVSAEALESQELcGTEVPSVPEHGRK | 253 | 3 | 1 | 1 |
| SLTFnETYQDISELVYGAK | 254 | 29 | 28 | 28 |
| SPYEMFGDEEVmcLNGnWTEPPQcK | 255 | 31 | 31 | 27 |
| SPYYnVSDEISFHcYDGYTLR | 256 | 157 | 151 | 147 |
| SQILEGLGFnLTELSESDVHR | 257 | 20 | 18 | 20 |
| SRVYLQGLIDcYLFGnSSTVLEDSK | 258 | 1 | 2 | 2 |
| SRYPHKPEInSTTHPGADLQENFcR | 259 | 16 | 16 | 17 |
| SSVITLnTnAELFnQSDIVAHLLSSSSSVIDALQYK | 260 | 1 | 1 | 1 |
| STGKPTLYnVSLVMSDTAGTcY | 261 | 6 | 7 | 7 |
| SVQEIQATFFYFTPnKTEDTIFLR | 262 | 104 | 113 | 101 |
| SVVAPATDGGLnLTSTFLR | 263 | 1 | 1 | 1 |
| TEGRPDmKTELFSSScPGGImLnETGQGYQR | 264 | 2 | 2 | 4 |
| TELFSSScPGGImLnETGQGYQR | 265 | 12 | 12 | 12 |
| TEVSSnHVLIYLDKVSnQTLSLFFTVLQDVPVR | 266 | 7 | 10 | 8 |
| TEVSSnHVLIYLDKVSnQTLSLFFTVLQDVPVRDLKPAIVK | 267 | 2 | 2 | 2 |
| THTnISESHPnATFSAVGEASIcEDDWnSGER | 268 | 20 | 25 | 17 |
| TKPREEQYnSTYR | 269 | 1 | 1 | 1 |
| TLFcnASKEWDnTTTEcR | 270 | 1 | #N/A | 1 |
| TLnQSSDELQLSMGnAmFVK | 271 | 206 | 217 | 210 |
| TLYETEVFSTDFSnISAAK | 272 | 9 | 7 | 9 |
| TTTVQVPmMHQmEQYYHLVDmELncTVLQMDYSK | 273 | 10 | 9 | 9 |
| TVIRPFYLTnSSGVD | 274 | 3 | 4 | 2 |
| TVLTPATNHmGnVTFTIPANR | 275 | 26 | 19 | 27 |
| TVLTPATNHmGnVTFTIPAnREFK | 276 | 2 | 1 | 1 |
| TVVTYHIPQnSSLENVDSR | 277 | 1 | 1 | 1 |
| TYNVLDmKnTTcQDLQIEVTVK | 278 | 7 | 5 | 3 |
| VASVININPnTTHSTGScR | 279 | 3 | 3 | 2 |
| VcQDcPLLAPLnDTR | 280 | 25 | 29 | 30 |
| VcQDcPLLAPLnDTRVVHAAK | 281 | 6 | 8 | 9 |
| VDKDLQSLEDILHQVEnK | 282 | 1 | 1 | #N/A |
| VEGSSSHLVTFTVLPLEIGLHNInFSLETWFGK | 283 | 1 | 1 | 1 |
| VEnTTVYYLVLDVQESDcSVLSR | 284 | 22 | 15 | 24 |
| VFHIHnESWVLLTPK | 285 | 4 | 3 | 3 |
| VFPLSLDSTPQDGNVVVAcLVQGFFPQEPLSVTWSESGQnVTAR | 286 | 12 | 12 | 9 |
| VGQLQLSHnLSLVILVPQNLK | 287 | 19 | 17 | 17 |
| VIDFncTTSSVSSALANTK | 288 | 18 | 16 | 18 |
| VIDFncTTSSVSSALAnTKDSPVLIDFFEDTER | 289 | 1 | 1 | 2 |
| VLSnNSDANLELInTWVAK | 290 | 54 | 39 | 33 |
| VLTLNLDQVDFQHAGnYScVASNVQGK | 291 | 2 | 1 | 1 |
| VLYLAAYncTLRPVSK | 292 | 7 | 9 | 8 |
| VPGnVTAVLGETLK | 293 | 1 | 1 | #N/A |
| VPMmLQSSTISYLHDSELPcQLVQmNYVGnGTVFFILPDK | 294 | 7 | 12 | 7 |
| VSAITLVSATSTTAnmTVGPEGK | 295 | 4 | 3 | 3 |
| VSEHIPVYQQEEnQTDVWTLLNGSK | 296 | 8 | 9 | 7 |
| VSEHIPVYQQEEnQTDVWTLLnGSKDDFLIYDR | 297 | 6 | 8 | 7 |
| VSLTnVSISDEGR | 298 | 1 | 1 | #N/A |
| VSnQTLSLFFTVLQDVPVR | 299 | 28 | 23 | 25 |
| VSnVScQASVSR | 300 | 1 | 2 | 1 |
| VSTVYANnGSVLQGTSVASVYHGK | 301 | 1 | 1 | 1 |
| VTAcHSSQPnATLYK | 302 | 7 | 7 | 7 |
| VTISGVYDLGDVLEEmGIADLFTNQAnFSR | 303 | 12 | 13 | 14 |
| VTQnLTLIEESLTSEFIHDIDR | 304 | 9 | 7 | 8 |
| VTQVYAEnGTVLQGSTVASVYK | 305 | 14 | 13 | 15 |
| VTQVYAEnGTVLQGSTVASVYKGK | 306 | 3 | 3 | 2 |
| VTWKPQGAPVEWEEETVTnHTLR | 307 | 1 | #N/A | #N/A |
| VVLHPnYSQVDIGLIK | 308 | 29 | 24 | 31 |
| VVLHPnYSQVDIGLIKLK | 309 | 1 | #N/A | 2 |
| VYIHPFHLVIHnESTcEQLAK | 310 | 14 | 13 | 15 |
| VYKPSAGnnSLYR | 311 | 9 | 7 | 9 |
| VYLQGLIDcYLFGnSSTVLEDSK | 312 | 5 | 8 | 6 |
| VYSGILnQSEIKEDTSFFGVQEIIIHDQYK | 313 | 1 | 1 | #N/A |
| WDPEVncSmAQIQLcPPPPQIPnSHnMTTTLNYR | 314 | 30 | 36 | 31 |
| WFSAGLASnSSWLR | 315 | 2 | 3 | 1 |
| WFYIASAFRNEEYnK | 316 | 3 | 3 | 3 |
| WnITmESYVVHTNYDEYAIFLTK | 317 | 12 | 11 | 9 |
| WNVNAPPTFHSEMMYDnFTLVPVWGK | 318 | 3 | 6 | #N/A |
| WVLTAAHcLLYPPWDKnFTENDLLVR | 319 | 14 | 15 | 14 |
| YAEDKFnETTEK | 320 | 7 | 9 | 8 |
| YFYnGTSmAcETFQYGGcmGnGNNFVTEK | 321 | 25 | 16 | 22 |
| YGNPNETQnnSTSWPVFK | 322 | 4 | 5 | 3 |
| YKGLnLTEDTYKPR | 323 | 2 | 2 | 2 |
| YLGnATAIFFLPDEGK | 324 | 115 | 102 | 103 |
| YLGnATAIFFLPDEGKLQHLEnELTHDIITK | 325 | 58 | 52 | 63 |
| YLHTAVIVSGTMLVFGGNTHnDTSmSHGAK | 326 | 2 | 3 | 3 |
| YnSQnQSNNQFVLYR | 327 | 14 | 10 | 11 |
| YnWSFIHcPAcQcnGHSK | 328 | 3 | 3 | #N/A |
| YPHKPEInSTTHPGADLQENFcR | 329 | 17 | 16 | 19 |
| YPPTVSmVEGQGEKnVTFWGRPLPR | 330 | 1 | 1 | #N/A |
| YQFNTNVVFSnnGTLVDR | 331 | 9 | 5 | 6 |
| YTcEEPYYYmEnGGGGEYHcAGnGSWVnEVLGPELPK | 332 | 12 | 10 | 8 |
| YTGnASALFILPDQDK | 333 | 16 | 15 | 17 |
| YTGnASALFILPDQDKmEEVEAmLLPETLK | 334 | 25 | 28 | 26 |
| YTGnASALFILPDQDKmEEVEAmLLPETLKR | 335 | 31 | 33 | 30 |

TABLE 3
Unique Formerly N-linked Glycopeptides of Human Serum Identified in
Three LC-MS/MS Replicates.

| | | Peptide Spectrum Match | | |
|---|---|---|---|---|
| | SEQ ID | Injection | Injection | Injection |
| Sequence | NO: | 1 | 2 | 3 |
| AAINKWVSnKTEGR | 336 | 1 | 1 | #N/A |
| AALAAFnAQNnGSnFQLEEISR | 4 | 114 | 120 | 119 |
| AATcINPLnGSVcERPAnHSAK | 5 | 1 | 1 | 1 |
| ADGTVnQIEGEATPVnLTEPAK | 6 | 20 | 19 | 19 |
| ADGTVNQIEGEATPVnLTEPAKLEVK | 7 | 12 | 11 | 13 |
| ADTHDEILEGLNFnLTEIPEAQIH | 8 | 10 | 7 | 8 |
| ADTHDEILEGLnFnLTEIPEAQIHEGFQELLR | 9 | 118 | 122 | 117 |
| AELSnHTRPVILVPGcLGnQLEAK | 10 | 2 | 3 | 2 |
| AFEnVTDLQWLILDHnLLEnSK | 11 | 15 | 17 | 18 |
| AFHYnVSSHGcQLLPWTQHSPHTR | 12 | 2 | 2 | 2 |
| AFITnFSMIIDGmTYPGIIK | 13 | 7 | 7 | 7 |
| AFITnFSMIIDGmTYPGIIKEK | 14 | 3 | 3 | 3 |
| AGAFLGLTNVAVmnLSGNcLR | 15 | 9 | 10 | 8 |
| AGLQAFFQVQEcnK | 16 | 5 | 5 | 7 |
| AHLnVSGIPcSVLLADVEDLIQQQISnDTVSPR | 17 | 2 | 1 | 1 |
| ALPQPQnVTSLLGcTH | 18 | 6 | 7 | 5 |
| ALYAWNNGHQILYnVTLFHVIR | 337 | 1 | #N/A | #N/A |
| AnLSSQALQmSLDYGFVTPLTSMSIR | 21 | 12 | 12 | 13 |
| APDKNVIFSPLSISTALAFLSLGAHnTTLTEILK | 22 | 9 | 9 | 9 |
| AQLLQGLGFnLTER | 23 | 20 | 19 | 18 |
| AQVIInITDVDEPPIFQQPFYHFQLK | 24 | 3 | 2 | 2 |
| AREDIFMETLKDIVEYYNDSnGSHVLQGR | 25 | 21 | 22 | 21 |
| AVLQLnEEGVDTAGSTGVTLnLTSKPIILR | 26 | 34 | 38 | 37 |
| AVnITSENLIDDVVSLIR | 27 | 11 | 12 | 10 |
| AYLLPAPPAPGnASESEEDR | 28 | 3 | 3 | 3 |
| cATPHGDnASLEATFVK | 29 | 3 | 3 | 3 |
| cGLVPVLAENYnK | 30 | 2 | 3 | 4 |
| cGncSLTTLKDEDFcK | 31 | 7 | 6 | 7 |
| cGncSLTTLKDEDFcKR | 32 | 7 | 8 | 7 |
| cIQAnYSLmENGK | 33 | 15 | 21 | 21 |
| cIQAnYSLmEnGKIK | 34 | 2 | 4 | 2 |
| cmWSSALnSLnLSFAGLEQVPK | 35 | 3 | 4 | 3 |
| cSDGWSFDATTLDDnGTmLFFK | 36 | 16 | 17 | 19 |
| DFVnASSKYEITTIHNLFR | 37 | 4 | 4 | 3 |
| DHEnGTGTNTYAALNSVYLMMNNQMR | 38 | 7 | 8 | 6 |
| DIVEYYnDSnGSHVLQGR | 39 | 34 | 36 | 37 |
| DKIcDLLVANNHFAHFFAPQnLTNmNK | 40 | 34 | 32 | 31 |
| DmTEVISSLENANYKDHEnGTGTnTYAALNSVYLMMNNQmR | 41 | 7 | 8 | 6 |
| DQcIVDDITYNVnDTFHK | 42 | 11 | 10 | 11 |
| DRQDGEEVLQcmPVcGRPVTPIAQnQTTLGSSR | 44 | 1 | #N/A | 1 |
| DSVSVVLGQHFFnR | 45 | 3 | 3 | 3 |
| DTAVFEcLPQHAmFGNDTITcTTHGnWTK | 46 | 32 | 29 | 32 |
| DTAVFEcLPQHAmFGnDTITcTTHGnWTKLPEcR | 47 | 11 | 9 | 10 |
| DVQIIVFPEDGIHGFnFTR | 48 | 8 | 8 | 7 |
| EDIFmETLKDIVEYYnDSNGSHVLQGR | 49 | 4 | 5 | 4 |
| EEQYNSTYRVVSVLTVLHQDWLnGKEYK | 51 | 1 | 1 | 1 |
| EGDHEFLEVPEAQEDVEATFPVHQPGnYScSYR | 52 | 19 | 16 | 17 |
| EGYSnISYIVVNHQGISSR | 53 | 10 | 9 | 9 |
| EHEAQSnASLDVFLGHTNVEELmK | 54 | 11 | 10 | 9 |
| EHEGAIYPDnTTDFQR | 55 | 25 | 25 | 26 |
| EHETcLAPELYNGnYSTTQK | 56 | 6 | 7 | 5 |
| EHYnLSAATcSPGQmcGHYTQVVWAK | 57 | 2 | 3 | 2 |
| ELDREVYPWYnLTVEAK | 58 | 1 | 1 | 1 |
| ELHHLQEQnVSNAFLDK | 59 | 25 | 28 | 27 |
| ELHHLQEQnVSnAFLDKGEFYIGSK | 60 | 101 | 100 | 94 |
| ELPGVcnETmmALWEEcKPcLK | 61 | 8 | 9 | 10 |
| EnLTAPGSDSAVFFEQGTTR | 62 | 17 | 16 | 16 |
| EQFcPPPPQIPNAQnMTTTVNYQDGEK | 338 | 1 | 1 | #N/A |
| ERSWPAVGncSSALR | 63 | 2 | 2 | 2 |
| EVnTSGFAPARPPPQPGSTTFWAWSVLR | 64 | 6 | 5 | 5 |
| EVSFLncSLDnGGcTHYcLEEVGWR | 65 | 6 | 7 | 5 |
| EVYPWYnLTVEAK | 66 | 2 | 2 | 2 |
| EWEKELHHLQEQnVSnAFLDKGEFYIGSK | 67 | 6 | 3 | 5 |
| EYESYSDFERnVTEK | 68 | 2 | 1 | 1 |
| FcRDnYTDLVAIQNK | 69 | 1 | 1 | 2 |
| FDFQGTcEYLLSAPcHGPPLGAEnFTVTVAnEHR | 70 | 1 | #N/A | 1 |
| FEDGVLDPDYPRnISDGFDGIPDnVDAALALPAHSYSGR | 71 | 8 | 7 | 7 |
| FEVDSPVYnATWSASLK | 72 | 4 | 4 | 3 |
| FGHSAVLHnSTMYVFGGFNSLLLSDILVFTSEQcDAHR | 73 | 5 | 7 | 5 |
| FHDVSESTHWTPFLnASVHYIR | 74 | 6 | 6 | 6 |
| FLNnGTcTAEGK | 75 | 1 | 1 | 3 |
| FnLTETSEAEIHQSFQHLLR | 78 | 183 | 177 | 182 |
| FNPGAESVVLSnSTLK | 79 | 2 | 2 | 1 |
| FnSSYLQGTNQITGR | 80 | 3 | 1 | 2 |
| FQSPAGTEALFELHNISVADSAnYScVYVDLKPPFGGSAPSER | 81 | 87 | 85 | 93 |
| FSDGLESnSSTQFEVK | 82 | 40 | 44 | 52 |
| FSDGLESnSSTQFEVKK | 83 | 1 | 3 | 3 |
| FSLLGHASIScTVEnETIGVWRPSPPTcEK | 84 | 68 | 71 | 73 |
| FSYSKnETYQLFLSYSSK | 85 | 8 | 8 | 10 |
| FVGTPEVnQTTLYQR | 86 | 1 | 2 | 1 |
| FVQAIcEGDDcQPPAYTYNnITcASPPEVVGLDLR | 87 | 6 | 4 | 6 |
| FVQGnSTEVAcHPGYGLPK | 88 | 2 | 3 | 4 |
| GAFISnFSmTVDGK | 89 | 11 | 10 | 9 |
| GcnDSDVLAVAGFALR | 90 | 3 | 4 | 3 |
| GcScFSDWQGPGcSVPVPAnQSFWTR | 91 | 3 | 5 | 4 |
| GcVLLSYLnETVTVSASLESVR | 92 | 124 | 135 | 132 |
| GDSGGPLVcmDAnnVTYVWGVVSWGEncGKPEFPGVYTK | 93 | 14 | 14 | 12 |
| GELnTSIFSSRPIDK | 339 | 1 | 1 | #N/A |
| GETHEQVHSILHFKDFVnASSK | 94 | 1 | 3 | 1 |
| GETHEQVHSILHFKDFVnASSKYEITTIHNLFR | 95 | 1 | 1 | 1 |
| GFGVAIVGnYTAALPTEAALR | 96 | 46 | 47 | 49 |
| GFLALYQTVAVnYSQPISEASR | 97 | 6 | 7 | 7 |
| GFYPSDIAVEWESSGQPEnnYnTTPPmLDSDGSFFLYSK | 340 | 1 | #N/A | #N/A |
| GGETAQSADPQWEQLnnKnLSmPLLPADFHK | 98 | 12 | 10 | 13 |
| GGNSnGALcHFPFLYNnHnYTDcTSEGR | 99 | 14 | 16 | 14 |
| GGNSNGALcHFPFLYnNHnYTDcTSEGRR | 100 | 1 | #N/A | 1 |
| GLKFnLTETSEAEIHQSFQHLLR | 102 | 85 | 79 | 86 |
| GLTFQQnASSmcGPDQDTAIR | 104 | 9 | 10 | 8 |
| GLTFQQnASSmcVPDQDTAIR | 105 | 9 | 9 | 9 |
| GmnLTVFGGTVTAFLGIPYAQPPLGR | 106 | 6 | 5 | 4 |
| GNEANYYSNATTDEHGLVQFSInTTnVmGTSLTVR | 107 | 143 | 138 | 134 |
| GNVAVTVSGHTcQHWSAQTPHTHnR | 108 | 6 | 7 | 9 |
| GSFPWQAKMVSHHnLTTGATLINEQWLLTTAK | 341 | 1 | #N/A | #N/A |
| GTAnTTTAGVPcQR | 110 | 3 | 3 | 2 |
| GTGnDTVLNVALLNVISNQEcNIK | 111 | 1 | 2 | 1 |
| GVTSVSQIFHSPDLAIRDTFVnASR | 112 | 5 | 5 | 3 |
| HAnWTLTPLK | 113 | 5 | 4 | 5 |
| HEEGHmLncTcFGQGR | 114 | 6 | 7 | 6 |
| HGIQYFnnNTQHSSLFmLNEVK | 115 | 25 | 22 | 29 |
| HGIQYFnnNTQHSSLFmLNEVKR | 116 | 12 | 14 | 14 |
| HGIQYFnnNTQHSSLFTLnEVK | 117 | 39 | 39 | 40 |
| HGIQYFnnNTQHSSLFTLNEVKR | 118 | 10 | 10 | 11 |
| HGVIISSTVDTYEnGSSVEYR | 119 | 9 | 11 | 10 |
| HLQmDIHIFEPQGISFLETESTFmTNQLVDALTTWQnK | 120 | 6 | 8 | 5 |
| HSHNNnSSDLHPHK | 342 | 1 | 4 | #N/A |
| HYLVSnISHDTVLQcHFTcSGK | 121 | 2 | 2 | 3 |
| HYTnSSQDVTVPcR | 122 | 14 | 13 | 13 |
| HYYIAAEEIIWnYAPSGIDIFTKEnLTAPGSDSAVFFEQGTTR | 123 | 8 | 8 | 11 |
| IADAHLDRVEnTTVYYLVLDVQESDcSVLSR | 124 | 65 | 68 | 68 |
| IcDLLVANNHFAHFFAPQnLTnMNK | 125 | 25 | 21 | 23 |
| IDSTGnVTNELR | 126 | 1 | 2 | 3 |
| IITILEEEmnVSVcGLYTYGKPVPGHVIVSIcR | 127 | 79 | 82 | 74 |
| INNDFNYEFYnSTWSYVK | 128 | 6 | 6 | 6 |
| IPcSQPPQIEHGTInSSR | 129 | 20 | 20 | 21 |
| ISEEnETTcYMGK | 130 | 14 | 15 | 14 |
| ISnSSDTVEcEcSENWK | 131 | 4 | 5 | 5 |
| ISnSSDTVEcEcSEnWKGEAcDIPHcTDncGFPHR | 132 | 5 | 4 | 3 |
| ITPNLAEFAFSLYRQLAHQSnSTNIFFSPVSIATAFAmLSLGTK | 133 | 2 | 1 | 2 |
| ITYSIVQTncSK | 134 | 5 | 8 | 12 |
| ITYSIVQTncSKEnFLFLTPDcK | 135 | 11 | 9 | 11 |
| IVGGTnSSWGEWPWQVSLQVK | 136 | 8 | 8 | 8 |
| IVLDPSGSMnIYLVLDGSDSIGASnFTGAK | 137 | 135 | 132 | 134 |
| IYPGVDFGGEELnVTFVK | 138 | 5 | 7 | 7 |
| IYSGILnLSDITK | 139 | 6 | 7 | 7 |
| lYSnHSALESLALIPLQAPLK | 140 | 4 | 3 | 3 |
| KAFITnFSMIIDGmTYPGIIK | 141 | 4 | 4 | 4 |
| KAFITnFSMIIDGmTYPGIIKEK | 142 | 6 | 6 | 4 |
| KcGncSLTTLKDEDFcK | 143 | 1 | 1 | 1 |
| KDFEDLYTPVnGSIVIVR | 144 | 2 | 1 | 2 |
| KEDALnETR | 343 | 1 | 1 | #N/A |
| KEHETcLAPELYNGnYSTTQK | 145 | 10 | 11 | 12 |
| KIVLDPSGSMnIYLVLDGSDSIGASnFTGAK | 146 | 36 | 49 | 42 |
| KLHINHNnLTESVGPLPK | 147 | 8 | 8 | 6 |
| KLINDYVKnGTR | 148 | 2 | 2 | 2 |
| KLPPGLLAnFTLLR | 149 | 4 | 5 | 5 |
| KLSSWVLLmKYLGnATAIFFLPDEGK | 344 | 1 | #N/A | #N/A |
| KnQSVNVFLGHTAIDEMLK | 150 | 6 | 4 | 3 |
| KQVHFFVnASDVDNVK | 151 | 6 | 6 | 5 |
| KVcQDcPLLAPLnDTR | 152 | 14 | 14 | 12 |
| LAGKPTHVnVSVVMAEVDGTcY | 153 | 54 | 56 | 64 |
| LAnLTQGEDQYYLR | 154 | 14 | 15 | 15 |
| LATALSLSNKFVEGSHnSTVSLTTK | 155 | 2 | 2 | 2 |
| LDAPTNLQFVnETDSTVLVR | 156 | 9 | 8 | 9 |
| LDPVSLQTLQTWnTSYPK | 157 | 2 | 2 | 1 |
| LDREnISEYHLTAVIVDK | 158 | 1 | 1 | 1 |
| LDREnISEYHLTAVIVDKDTGEnLETPSSFTIK | 159 | 1 | 1 | 1 |
| LEDLEVTGSSFLnLSTnIFSnLTSLGK | 160 | 8 | 10 | 12 |
| LEPVHLQLQcMSQEQLAQVAAnATK | 161 | 11 | 12 | 12 |
| LETTVnYTDSQRPIcLPSK | 162 | 3 | 2 | 3 |
| LFGDKSLTFnETYQDISELVYGAK | 163 | 5 | 6 | 4 |
| LGAcnDTLQQLMEVFK | 164 | 32 | 31 | 34 |
| LGAcnDTLQQLmEVFKFDTISEK | 165 | 10 | 10 | 9 |
| LGAcnDTLQQLMEVFKFDTISEKTSDQIHFFFAK | 166 | 4 | 4 | 3 |
| LGHcPDPVLVnGEFSSSGPVnVSDK | 167 | 8 | 9 | 9 |
| LGSFEGLVnLTFIHLQHNR | 168 | 9 | 11 | 13 |
| LGSLQELFLDSnnISELPPQVFSQLFcLER | 169 | 4 | 2 | 3 |
| LGSYPVGGnVSFEcEDGFILR | 170 | 6 | 6 | 5 |
| LGTSLSSGHVLMnGTLK | 171 | 8 | 8 | 8 |
| LHINHNnLTESVGPLPK | 172 | 10 | 14 | 11 |
| LKELPGVcnETMmALWEEcKPcLK | 173 | 14 | 12 | 11 |
| LLLSQLDSHPSHSAVVnWTSYASSIEALSSGNK | 174 | 1 | 1 | 1 |
| LNAENnATFYFK | 175 | 75 | 103 | 108 |
| LnDTLDYEcHDGYESnTGSTTGSIVcGYnGWSDLPIcYER | 176 | 17 | 19 | 14 |
| LNVEAAnWTVR | 177 | 4 | 3 | 4 |
| LPPGLLAnFTLLR | 179 | 4 | 4 | 3 |
| LPTQnITFQTESSVAEQEAEFQSPK | 180 | 28 | 28 | 30 |
| LPYQGnATmLVVLmEK | 181 | 1 | 2 | 2 |
| LQAILGVPWKDKncTSR | 182 | 12 | 13 | 13 |
| LQAPLnYTEFQKPIcLPSK | 183 | 7 | 7 | 7 |
| LQNnENnIScVER | 184 | 6 | 7 | 7 |
| LSDLSInSTEcLHVHcR | 185 | 81 | 100 | 84 |
| LSHnELADSGIPGnSFnVSSLVELDLSYNK | 186 | 19 | 23 | 26 |
| LSLHRPALEDLLLGSEAnLTcTLTGLR | 187 | 88 | 88 | 92 |
| LSSWVLLmKYLGnATAIFFLPDEGK | 188 | 1 | #N/A | 1 |
| LSVDKDQYVEPEnVTIQcDSGYGVVGPQSITcSGnR | 189 | 4 | 4 | 5 |
| LTDTIcGVGnmSAnASDQER | 190 | 3 | 5 | 5 |
| LVSANRLFGDKSLTFnETYQDISELVYGAK | 191 | 2 | 3 | 3 |
| LYHFLLGAWSLnATELDPcPLSPELLGLTK | 192 | 19 | 20 | 15 |
| LYLGSNnLTALHPALFQnLSK | 193 | 11 | 10 | 12 |
| mAGKPTHInVSVVmAEADGTcY | 194 | 3 | 4 | 2 |
| mAGKPTHVnVSVVmAEVDGTcY | 195 | 4 | 3 | 3 |
| mAWPEDHVFISTPSFnYTGR | 196 | 4 | 4 | 6 |
| MDGASnVTcInSR | 197 | 23 | 24 | 25 |
| MLLTFHTDFSNEEnGTImFYK | 198 | 1 | 1 | 1 |
| mLnTSSLLEQLnEQFNWVSR | 200 | 27 | 27 | 26 |
| mPSQAPTGNFYPQPLLnSSmcLEDSR | 201 | 2 | 4 | 4 |
| mVSHHnLTTGATLInEQWLLTTAK | 204 | 453 | 443 | 443 |
| MVSHHnLTTGATLInEQWLLTTAKNLFLnHSEnATAK | 205 | 12 | 11 | 10 |
| mVTAFTTccTLSEEFAcVDNLADLVFGELcGVNEnR | 206 | 2 | 3 | 2 |
| NAHGEEKEnLTAR | 207 | 1 | #N/A | 1 |
| NcGVncSGDVFTALIGEIASPnYPKPYPEnSR | 208 | 7 | 8 | 8 |
| NEEYnKSVQEIQATFFYFTPnKTEDTIFLR | 210 | 11 | 11 | 12 |
| nEMLEIQVFNYSKVFSnK | 211 | 2 | 1 | 2 |
| nGTGHGnSTHHGPEYmR | 212 | 4 | 6 | 2 |
| NHPnITFFVYVSnFTWPIK | 213 | 3 | 4 | 4 |
| nISDGFDGIPDNVDAALALPAHSYSGR | 214 | 2 | 2 | 2 |
| NLASRPYTFHSHGITYYKEHEGAIYPDnTTDFQR | 215 | 5 | 4 | 5 |
| NLFLnHSENATAK | 216 | 248 | 255 | 243 |
| NLFLnHSEnATAKDIAPTLTLYVGK | 217 | 9 | 11 | 11 |
| NLFLnHSEnATAKDIAPTLTLYVGKK | 218 | 2 | 3 | 2 |
| NmASRPYSIYPHGVTFSPYEDEVnSSFTSGR | 345 | 1 | #N/A | #N/A |
| nnATVHEQVGGPSLTSDLQAQSK | 219 | 41 | 45 | 51 |
| NnmSFVVLVPTHFEWnVSQVLAnLSWDTLHPPLVWERPTK | 220 | 2 | 1 | 2 |
| NPPmGGNVVIFDTVITnQEEPYQnHSGR | 221 | 6 | 5 | 5 |
| NPVGLIGAEnATGETDPSHSK | 222 | 10 | 10 | 11 |
| nQALnLSLAYSFVTPLTSMVVTKPDDQEQSQVAEKPmEGESR | 223 | 8 | 9 | 9 |
| NSVLnSSTAEHSSPYSEDPIEDPLQPDVTGIR | 224 | 2 | 4 | 3 |
| NVIFSPLSISTALAFLSLGAHnTTLTEILK | 225 | 9 | 10 | 11 |
| QDQcIYnTTYLnVQR | 226 | 160 | 164 | 181 |
| QDQcIYnTTYLNVQREnGTISR | 227 | 5 | 4 | 5 |
| QEDLSVGSVLLTVnATDPDSLQHQTIR | 228 | 1 | 2 | 1 |
| QGGVnATQVLIQHLR | 229 | 1 | #N/A | 1 |
| QInSSISGNLWDKDQR | 230 | 1 | 1 | 3 |
| QLAHQSnSTnIFFSPVSIATAFAMLSLGTK | 231 | 83 | 86 | 92 |
| QLDmLDLSnNSLASVPEGLWASLGQPnWDmR | 232 | 18 | 15 | 16 |
| QLEEFLnQSSPFYFWmnGDR | 233 | 28 | 27 | 27 |
| QLEEFLnQSSPFYFWmnGDRIDSLLEnDR | 234 | 11 | 11 | 11 |
| QLVEIEKVVLHPnYSQVDIGLIK | 235 | 6 | 5 | 5 |
| QNESHnFSGDIALLELQHSIPLGPNVLPVcLPDnETLYR | 236 | 5 | 6 | 5 |
| QnQcFYnSSYLnVQR | 237 | 17 | 18 | 18 |
| QPQAGLSQAnFTLGPVSR | 238 | 1 | 1 | 1 |
| QQQHLFGSnVTDcSGNFcLFR | 239 | 159 | 161 | 160 |
| QVHFFVnASDVDNVK | 240 | 12 | 11 | 12 |
| QVLFLDTVYGncSTHFTVK | 241 | 5 | 4 | 4 |
| QVQVLQnLTTTYEIVLWQPVTADLIVK | 242 | 2 | 3 | 2 |
| REGDHEFLEVPEAQEDVEATFPVHQPGnYScSYR | 243 | 12 | 14 | 12 |
| RHEEGHmLncTcFGQGR | 244 | 3 | 4 | 1 |
| RNPPmGGNVVIFDTVITnQEEPYQnHSGR | 245 | 13 | 12 | 12 |
| SDHGSSIScQPPAEIPGYLPADTVHLAVEFFNLTHLPAnLLQGASK | 246 | 9 | 9 | 10 |
| SHAASDAPEnLTLLAETADAR | 247 | 1 | 1 | 1 |
| SHEIWTHScPQSPGnGTDASH | 248 | 1 | 2 | 2 |
| SIPAcVPWSPYLFQPnDTcIVSGWGR | 249 | 14 | 12 | 13 |
| SKPTVSSSmEFKYDFnSSmLYSTAK | 250 | 1 | 1 | 1 |
| SKWnITmESYVVHTNYDEYAIFLTK | 251 | 19 | 18 | 19 |
| SLGnVnFTVSAEALESQELcGTEVPSVPEHGR | 252 | 146 | 155 | 157 |
| SLGnVnFTVSAEALESQELcGTEVPSVPEHGRK | 253 | 3 | 3 | 3 |
| SLTFnETYQDISELVYGAK | 254 | 34 | 28 | 29 |
| SPYEMFGDEEVmcLNGnWTEPPQcK | 255 | 28 | 30 | 31 |
| SPYYnVSDEISFHcYDGYTLR | 256 | 153 | 152 | 157 |
| SQILEGLGFnLTELSESDVHR | 257 | 20 | 21 | 20 |
| SRVYLQGLIDcYLFGnSSTVLEDSK | 258 | 2 | 2 | 1 |
| SRYPHKPEInSTTHPGADLQENFcR | 259 | 13 | 14 | 16 |
| STGKPTLYnVSLVMSDTAGTcY | 261 | 7 | 6 | 6 |
| SVQEIQATFFYFTPnKTEDTIFLR | 262 | 95 | 96 | 104 |
| SVTLQIYnHSLTLSAR | 346 | 1 | 1 | #N/A |
| SWPAVGncSSALR | 347 | 1 | #N/A | #N/A |
| TEGRPDmKTELFSSScPGGImLnETGQGYQR | 264 | 3 | 2 | 2 |
| TELFSSScPGGImLnETGQGYQR | 265 | 11 | 13 | 12 |
| TEVSSnHVLIYLDKVSnQTLSLFFTVLQDVPVR | 266 | 7 | 8 | 7 |
| TEVSSnHVLIYLDKVSnQTLSLFFTVLQDVPVRDLKPAIVK | 267 | 3 | 3 | 2 |
| THTnISESHPnATFSAVGEASIcEDDWnSGER | 268 | 17 | 16 | 20 |
| TKPREEQYnSTYR | 269 | 1 | 2 | 1 |
| TLFcnASKEWDnTTTEcR | 270 | 1 | #N/A | 1 |
| TLnQSSDELQLSmGnAmFVK | 271 | 184 | 190 | 206 |
| TLYETEVFSTDFSnISAAK | 272 | 8 | 9 | 9 |
| TTTVQVPmMHQmEQYYHLVDmELncTVLQMDYSK | 273 | 8 | 8 | 10 |
| TVIRPFYLTnSSGVD | 274 | 3 | 2 | 3 |
| TVLTPATNHmGnVTFTIPAnR | 275 | 25 | 22 | 26 |
| TVLTPATNHmGnVTFTIPAnREFK | 276 | 2 | 3 | 2 |
| TVVTYHIPQnSSLENVDSR | 277 | 1 | 1 | 1 |
| TYnVLDmKnTTcQDLQIEVTVK | 278 | 3 | 3 | 7 |
| VASVININPnTTHSTGScR | 279 | 2 | 3 | 3 |
| VcQDcPLLAPLnDTR | 280 | 24 | 29 | 25 |
| VcQDcPLLAPLnDTRVVHAAK | 281 | 6 | 6 | 6 |
| VDKDLQSLEDILHQVEnK | 282 | 1 | 1 | 1 |
| VEGSSSHLVTFTVLPLEIGLHNInFSLETWFGK | 283 | 1 | 1 | 1 |
| VEnTTVYYLVLDVQESDcSVLSR | 284 | 23 | 24 | 22 |
| VFHIHnESWVLLTPK | 285 | 2 | 3 | 4 |
| VFPLSLDSTPQDGNVVVAcLVQGFFPQEPLSVTWSESGQnVTAR | 286 | 13 | 15 | 12 |
| VGQLQLSHnLSLVILVPQNLK | 287 | 19 | 19 | 19 |
| VIDFncTTSSVSSALAnTK | 288 | 18 | 18 | 18 |
| VIDFncTTSSVSSALAnTKDSPVLIDFFEDTER | 289 | 2 | 1 | 1 |
| VKPnPPHNLSVINSEELSSILK | 348 | 1 | 1 | #N/A |
| VLSNNSDAnLELINTWVAK | 290 | 51 | 50 | 54 |
| VLTLNLDQVDFQHAGnYScVASNVQGK | 291 | 2 | #N/A | 2 |
| VLYLAAYncTLRPVSK | 292 | 8 | 8 | 7 |
| VPGnVTAVLGETLK | 293 | 1 | 1 | 1 |
| VPMMLQSSTISYLHDSELPcQLVQMnYVGnGTVFFILPDK | 294 | 11 | 9 | 7 |
| VPMMLQSSTISYLHDSELPcQLVQMNYVGnGTVFFILPDKGK | 349 | 2 | #N/A | #N/A |
| VSAITLVSATSTTAnmTVGPEGK | 295 | 3 | 3 | 4 |
| VSEHIPVYQQEEnQTDVWTLLNGSK | 296 | 7 | 7 | 8 |
| VSEHIPVYQQEEnQTDVWTLLnGSKDDFLIYDR | 297 | 8 | 7 | 6 |
| VSLTnVSISDEGR | 298 | 1 | 1 | 1 |
| VSnQTLSLFFTVLQDVPVR | 299 | 30 | 27 | 28 |
| VSnQTLSLFFTVLQDVPVRDLKPAIVK | 350 | 1 | 1 | #N/A |
| VSnVScQASVSR | 300 | 1 | 2 | 1 |
| VSTVYANnGSVLQGTSVASVYHGK | 301 | 1 | 1 | 1 |
| VTAcHSSQPnATLYK | 302 | 8 | 8 | 7 |
| VTISGVYDLGDVLEEmGIADLFTNQAnFSR | 303 | 14 | 15 | 12 |
| VTQnLTLIEESLTSEFIHDIDR | 304 | 9 | 9 | 9 |
| VTQVYAEnGTVLQGSTVASVYK | 305 | 12 | 15 | 14 |
| VTQVYAEnGTVLQGSTVASVYKGK | 306 | 2 | 3 | 3 |
| VTWKPQGAPVEWEEETVTnHTLR | 307 | 1 | 1 | 1 |
| VVLHPnYSQVDIGLIK | 308 | 32 | 29 | 29 |
| VVLHPnYSQVDIGLIKLK | 309 | 1 | 1 | 1 |
| VYIHPFHLVIHnESTcEQLAK | 310 | 11 | 13 | 14 |
| VYKPSAGnNSLYR | 311 | 6 | 7 | 9 |
| VYLQGLIDcYLFGnSSTVLEDSK | 312 | 7 | 8 | 5 |
| VYSGILnQSEIK | 351 | 1 | 1 | #N/A |
| WDPEVncSmAQIQLcPPPPQIPnSHnMTTTLNYR | 314 | 31 | 31 | 30 |
| WFSAGLASnSSWLR | 315 | 3 | 3 | 2 |
| WFYIASAFRNEEYnK | 316 | 2 | 3 | 3 |
| WnITmESYVVHTNYDEYAIFLTK | 317 | 12 | 14 | 12 |
| WNPcLEPHRFnDTEVLQR | 352 | 2 | 1 | #N/A |
| WNVNAPPTFHSEMMYDnFTLVPVWGK | 318 | 6 | 5 | 3 |
| WVLTAAHcLLYPPWDKnFTEnDLLVR | 319 | 14 | 15 | 14 |
| YAEDKFnETTEK | 320 | 5 | 6 | 7 |
| YFYnGTSmAcETFQYGGcmGnGNNFVTEK | 321 | 21 | 26 | 25 |
| YGNPNETQnnSTSWPVFK | 322 | 3 | 4 | 4 |
| YKGLnLTEDTYKPR | 323 | 2 | 2 | 2 |
| YLGnATAIFFLPDEGK | 324 | 102 | 106 | 115 |
| YLGnATAIFFLPDEGKLQHLEnELTHDIITK | 325 | 58 | 60 | 58 |
| YLHTAVIVSGTMLVFGGnTHnDTSmSHGAK | 326 | 2 | 3 | 2 |
| YnSQNQSnNQFVLYR | 327 | 10 | 8 | 14 |
| YnWSFIHcPAcQcNGHSK | 328 | 2 | 4 | 3 |
| YPHKPEInSTTHPGADLQENFcR | 329 | 16 | 17 | 17 |
| YPPTVSmVEGQGEKnVTFWGRPLPR | 330 | 1 | #N/A | 1 |
| YQFNTNVVFSNnGTLVDR | 331 | 8 | 6 | 9 |
| YTcEEPYYYmEnGGGGEYHcAGnGSWVnEVLGPELPK | 332 | 11 | 12 | 12 |
| YTGnASALFILPDQDK | 333 | 17 | 16 | 16 |
| YTGnASALFILPDQDKmEEVEAmLLPETLK | 334 | 26 | 27 | 25 |
| YTGnASALFILPDQDKmEEVEAmLLPETLKR | 335 | 31 | 33 | 31 |
| YTTFEYPnTINFScNTGFYLNGADSAK | 353 | 2 | #N/A | #N/A |

TABLE 4
Reproducibility of Glycopeptide
Isolations Using a Hydrazide Tip

| | Mean CV (%) | |
|---|---|---|
| | Between | Between |
| No. of | Injections | Isolations |
| Total PSMs | (n = 3) | (n = 3) |
| PSM >= 150 | 4.53 | 6.32 |
| 150 > PSM >= 60 | 6.27 | 11.36 |
| 60 > PSM >= 30 | 8.57 | 9.98 |
| 30 > PSM >= 15 | 11.53 | 17.01 |
| 15 > PSM | 21.55 | 28.1 |

### Peptide Isolation by Conjugation to Amino-Linking Beads

Several different glycoproteins were conjugated to amino-linking beads, the proteins were digested into peptides using the presently disclosed methods with amino-reactive tips and the peptides were used for global proteomics analysis.

Specifically, for the tube samples, casein was coupled to amino-linking beads at pH 10 for 4 h, reduced with NaCNBH₄ at pH 7 for 4 h, and the reaction sites on the beads were blocked with 1M Tris-HCl at pH 7 in the presence of NaCNBH₄ for 30 min. Then, the beads were denatured with 8M urea, reduced with TCEP, alkylated with IAA and digested with trypsin overnight.

Table 5 shows that conjugation of the amino-linking beads to the protein was most effective at pH 10.

TABLE 5
Efficiency of Amino-linking Beads at Different pH
Values and Capacity

| | Protein | | | | |
|---|---|---|---|---|---|
| | Loading (μg) | | Protein Conc. | | |
| | Per 50 μL | Volume | (mg/mL) | | % |
| pH | beads | (μL) | Before | After | Coupled |
| 10 | ~200 | 400 | 0.571 | undetectable | 100 |
| | ~350 | 400 | 0.879 | undetectable | 100 |
| | ~680 | 400 | 1.703 | 0.331 | 80.56 |
| 7 | ~200 | 400 | 0.46 | 0.204 | 55.65 |

### Example 2

### Tissue Proteomics by Mass Spectrometry: Elimination of OCT Interference Using Chemical Immobilization of Proteins for Peptide Extraction

Tissue proteomics are important for the identification of disease biomarkers, treatment targets and help in the understanding of the pathological characteristics of tissues. Tissues are commonly stored in an embedding medium like optimal cutting temperature compound (OCT) in the freezer or formalin-fixed and paraffin-embedded (FFPE) at room temperature in order to maintain the tissue morphology for histology evaluation. Currently, most of the tissue proteomic studies are performed on frozen tissues or FFPE embedded tissues. Due to the malicious effect of OCT to the mass spectrometer, only a handful of proteomics studies have been performed on OCT embedded tissues (Asomugha et al.; Somiari et al., 2003; Nirmalan et al.; Palmer-Toy et al., 2005; Scicchitano et al., 2009). OCT embedded tissues are studied using either two-dimensional gel electrophoresis (2D DIGE) technology or shot gun proteomics using LC-MS/MS. 2D DIGE could separate proteins from OCT; however, most of the LC-MS/MS studies of OCT embedded tissue had OCT contamination resulting in fewer protein identifications (Nirmalan et al.; Palmer-Toy et al., 2005; Scicchitano et al., 2009).

Tissue proteins play important roles in biological processes. Quantitative analysis of tissue proteins and their modifications such as phosphorylation, glycosylation, acetylation, is the key to the understanding of molecular mechanism that differentiates between normal and disease states. The disease-specific proteins from tissues can also be used as biomarkers for the diagnosis of diseases or as new drug targets for drug development as therapeutics (Zhang et al., 2007). In the diseased state, tissue secretes or sheds disease-specific proteins into the body fluids such as serum, which can be used as biomarkers. However, the excreted proteins from a diseased tissue have higher concentration at the tissue site and become diluted by mixing with other proteins from other tissues in serum (Zhang et al., 2007; Li et al., 2008). An example was shown in the process of detecting prostate cancer proteins in serum using TOF/TOF (Tian et al., 2008).

Traditionally, tissue proteins are analyzed using immunoassays, which rely on the development of high quality antibodies. Advances in mass spectrometry (MS) and high performance liquid chromatography (HPLC) systems have led to the blossoming of proteomics (Bantscheff et al., 2007). Increases in sensitivity, resolution, and speed of the mass spectrometers have led to the rapid identification of large numbers of proteins with high confidence, making the analysis of complex samples such as tissue possible. Tissue proteome, located at the primary site of pathology, helps to understand the molecular mechanism of diseases and providing a window of opportunity to identify potential biomarkers and therapeutic targets.

Tissue proteomics requires tissues to be stored by snap freezing. However, flash frozen tissues without embedding medium are difficult to section thereby making histopathology or immunohistochemistry evaluation difficult. Instead, tissues are embedded in optimal cutting temperature medium (OCT) or formalin-fixed and paraffin-embedded (FFPE) to retain its morphology (Turbett and Sellner, 1997). FFPE embedded tissues have been recently explored by various groups for proteomics analysis (Ralton and Murray, 2011; Vincenti and Murray, 2013). However, FFPE tissues during formalin fixation undergo extensive crosslinking between protein/DNA/RNA with methylene bridges creating inter and intra crosslinking of proteins (Turbett and Sellner, 1997; Magdeldin and Yamamoto, 2012). Some modifications of peptides in proteomics analysis of FFPE tissues are Metylol derivatives, Schiff bases and methylene bridges (Magdeldin and Yamamoto, 2012). Time span during FFPE process and storage can also lead to different levels of protein degradation and protein modifications. In contrast, OCT embedded tissues are instantly stored at freezers for histological studies; therefore, the protein contents are likely maintained and are representative of the tissue proteome.

However, the proteomic analysis of OCT-embedded tissues is difficult. OCT contains water soluble synthetic polymers and is widely used for embedding tissues for storage. OCT can compete with peptides for ionization during mass spectrometry analysis (Setou, 2010). OCT can also generate ion suppression in Matrix Assisted Laser Desorption and Ionization (MALDI) mass spectrometry and ionization competition in Electron spray ionization (ESI) mass spectrometry (Chaurand et al., 2004). In addition, OCT will create deleterious effect on the peptide chromatographic separation required for tissue proteomics. OCT has high affinity to reverse phase stationary medium commonly used in shotgun proteomics. OCT competes with peptides to bind to the column and prevails upon peptides for binding onto the C18 reverse phase column. OCT also decreases sensitivity of detection due to overlap with peptides during elution. For LC-MS/MS analysis of tissues, it is necessary to remove OCT from the sample.

In this study, a method is described using chemical immobilization of proteins for peptide extraction (CIPPE) from OCT-embedded tissues for tissue proteomic analysis. In this method, proteins are chemically immobilized onto solid support, which allows for sample cleaning and OCT removal by extensive washing before the peptides and modified peptides (glycopeptides) are released from the solid support using proteolysis. The method was applied to study the impact of OCT on tissue proteomics and glycoproteomics.

### Materials and Methods

Materials:

Human fetuin, dithiotheritol (DTT), and iodoacetamide were purchased from Sigma Aldrich (St. Louis, Mo.). Rapigest was purchased from Waters (Milford, Mass.). Protein estimation BCA kit, sodium cyanoborohydride, and Aminolink coupling resin was purchased from (Thermo Fisher Scientific Inc., Rockford, Ill.). Sequencing grade trypsin was purchased from Promega (Madison, Wis.). iTRAQ 4-plex reagents were purchased from AB Sciex (Framingham, Mass.). PNGase F was obtained from New England Biolabs (Ipswich, Mass.).

Protein Extraction:

Mouse kidney tissue was collected from NIH01a mice and snap frozen in Dr. Kemp's laboratory of Fred Hutchinson Cancer Research Cancer (Tian et al., 2010; Tian et al., 2009). Mouse kidney tissue was cut into two pieces. One was embedded in OCT followed by storage at −80° C. The second piece was stored as fresh-frozen tissue. OCT embedded or frozen mouse kidney tissues was lysed in 500 μL of pH 10 tissue lysis buffer (100 mM sodium citrate and 50 mM sodium carbonate in 2% SDS) by vortexing for 2-3 min and sonicating for 4 min in an ice bath to homogenize the tissues. After the tissues were homogenized, BCA was used to estimate the protein concentration.

Chemical Immobilization of Proteins to Beads:

Proteins were immobilized on to amino-link beads using previously described protocol (Yang et al, submitted to MCP). Briefly, amino-link resin (800 μL) was loaded onto snap-cap spin-column, and centrifuged at 2000 g for 1 minute. Resin was washed with 800 μL of pH 10 buffer (sodium citrate 100 mM and sodium carbonate 50 mM buffer) followed by centrifugation. The washing step was repeated twice. The sample in pH 10 buffer 10 (1 mg/200 microliter sample to beads ratio) was loaded onto amino-link resin. Volume was adjusted to 850 μL using pH 10 buffer.

Sample-resin mixture was incubated at room temperature overnight on a mixer. The mixture was centrifuged at 2000 g to remove any unbound protein. Resin was rinsed by 1×PBS buffer (Sigma-Aldrich; pH 7.4; 450 μL) three times. 50 mM sodium cyanoborohydride in PBS (400 μL) was added to resin (spin-column capped during each incubation step). After a four hour incubation, supernatant was removed via centrifugation (2000 g) and 400 μL of 1 M Tris-HCl (pH 7.6) in the presence of 50 mM sodium cyanoborohydride was added to block any un-reacted aldehyde sites of resin. The blocking process was terminated after 1 hour. Then, the beads were washed with PBS twice, 1.5M of NaCl twice, and water three times.

Peptide Extraction by Proteolysis:

Proteins bound on the beads were treated with 10 mM DTT in 50 mM ammonium bicarbonate for 30 mins at 60° C. followed by a wash with 50 mM ammonium bicarbonate. Afterwards, the beads were treated for 1 hr with 15 mM iodoacetamide in 50 mM ammonium bicarbonate in dark. Finally, proteins were digested using 1:50 trypsin to protein ratio in presence of 0.1% rapigest with 50 mM ammonium bicarbonate. The proteins were digested at 37° C. overnight. The released peptides were collected from the supernatant of the beads and the following wash step of the beads with water.

Ammonium bicarbonate was evaporated using freeze drying before iTRAQ labeling. iTRAQ labeling was performed according to manufactures protocol.

In-Solution Digestion:

Human serum albumin (HSA) protein with and without OCT was incubated with 10 mM DTT at 60° C. for 1 hr, and alkylated with 30 min incubation 10 mM iodoacetamide in dark at room temp. Finally, the pH of the solution was adjusted to the 7.5 with 50 mM NH₄HCO₃. Protein was enzymatically digested with trypsin using 1:50 trypsin to protein ratio with incubation overnight at 37° C.

Mass Spectrometric Analysis of Peptides Using Direct infusion to TSQ Quantum:

A TSQ Quantum Ultra (Thermo scientific, Rockford, Ill.) with electrospray ionization source was used for analysis of peptides from HSA using direct infusion. Flow rate was set at 5 μL/min. Peptides were scanned from m/z 300 to 1000 at voltage of 3000 V and capillary temperature 180° C. was used for the spray.

N-Glycopeptide Enrichment:

N-linked glycopeptides were isolated from 90% of peptides of the iTRAQ labeled sample. Samples described above were treated using SPEG method (Tian et al, 2007). The enriched N-linked glycopeptides were concentrated by C18 columns and fractionated using basic reverse phase into 12 fractions and analyzed using LC-MS/MS.

High-pH RPLC Fractionation:

Fifty μg iTRAQ labeled peptides were submitted to high-pH RPLC fractionation with a 1200 Infinity LC (Agilent Technology, Santa Clara, Calif.) and a 4.6×100 mm BEH130-C-18 column (Waters, Milford, Mass.). Samples were adjusted to a basic pH using 1% ammonium hydroxide, and injected in 2 mls of solvent A 7 mM tri-ethyl ammonium bicarbonate (TEAB). Solvent B is 7 mM TEAB, 90% acetonitrile.

The separation gradient was set as following: 0% B for 18 min, 0 to 31% B in 42 min, 31 to 50% B in 10 min, 75 to 100% B in 15 mM, and 100% B for an additional 10 min. Ninety-six fractions were collected along with the LC separation and were concatenated into 24 fractions by combining fractions 1, 25, 49, 73, and so on. For glycoproteomic analysis, glycopeptides were concatenated into 12 fractions by combining every 13^{th} fraction. The samples were dried in a Speed-Vac and stored at −80° C. until LC-MS/MS analysis.

LC-MS/MS Analysis:

Dionex Ultimate 3000 RELCnano system (Thermo Scientific, Rockford, Ill.) was used with a 75 μm×15 cm Acclaim PepMap100 separating column (Thermo Scientific, Rockford, Ill.). Peptides were separated using a flow rate of 300 nL/min with mobile phase A 0.1% formic acid in water and B consisting of 0.1% formic acid 95% acetonitrile. The gradient profile was set as follows: 4-35% B in 70 min, 35-95% B in 5 min. MS analysis was performed using an Orbitrap Velos Pro mass spectrometer (Thermo Scientific, Rockford, Ill.). The spray voltage was set at 2.2 kV. Orbitrap spectra were collected at a resolution of 60K followed by data-dependent HCD MS/MS (at a resolution of 7500, collision energy 45% and activation time 0.1 ms) of the ten most abundant ions. A dynamic exclusion time of 35 sec was used with a repeat count of 1.

Database Search:

Data generated using Orbitrap was searched using Proteome Discoverer 1.3 (Thermo Scientific, Rockford, Ill.) against IPI mouse database v3.30 with 56688 protein entries. Peptides were searched with two trypsin ends as protease, allowing only two missed cleavages. Search parameters used were 20 ppm precursor tolerance and 0.06 Da fragment ion tolerance, static modification of 4plex iTRAQ at N-terminus and Carbamidomethylation at Cysteine. Variable modification of oxidation at methionine and deamidation at aspargine and iTRAQ at lysine. Filters used for data analysis included peptide rank1, 2 peptides per protein, and 1% FDR threshold. For glycopeptides, NXS/T motif was used for further filtration of data.

Data Analysis of Removal of OCT from Human Serum Albumin (HAS):

Peaks were selected from ESI spectrum obtained from TSQ quantum with a threshold of 20% intensity of base peak intensity. Peaks were obtained from HSA protein digestion with OCT, without OCT, and with OCT followed by removal of OCT. Afterwards, they were aligned and compared. The comparison was performed between HSA, HSA with OCT, and HSA with OCT followed by OCT removal by CIPPE.

iTRAQ Data Analysis:

The Pearson's correlation coefficient of the peptide spectra obtained between replicated analyses of OCT embedded tissues (114, 115) using CIPPE was calculated to assess the reproducibility of the method to remove the OCT. Protein expressions in OCT embedded tissue (114, 115), and frozen tissue (116) were quantified and normalized by Proteome Discoverer 1.3. The log 2 ratios between replicates 114 and 115 were used as the “null” distribution, and the values for 5% cut-off (2.5th and 97.5th percentiles) of the histogram were selected as the thresholds for up- and down-expression thresholds. Similarly, the Pearson's correlation coefficient of the peptide spectra between the frozen tissue/OCT embedded tissues (116 and 114) was calculated to assess the impact of OCT embedding the tissue. The log 2 ratios between the frozen tissue/OCT embedded tissues (116 and 114) were compared with the up- and down-expression thresholds obtained in replicate analysis (“null” distribution). The same analysis protocol described above was applied to both the global proteomics data and the glycoproteomics data.

### Results

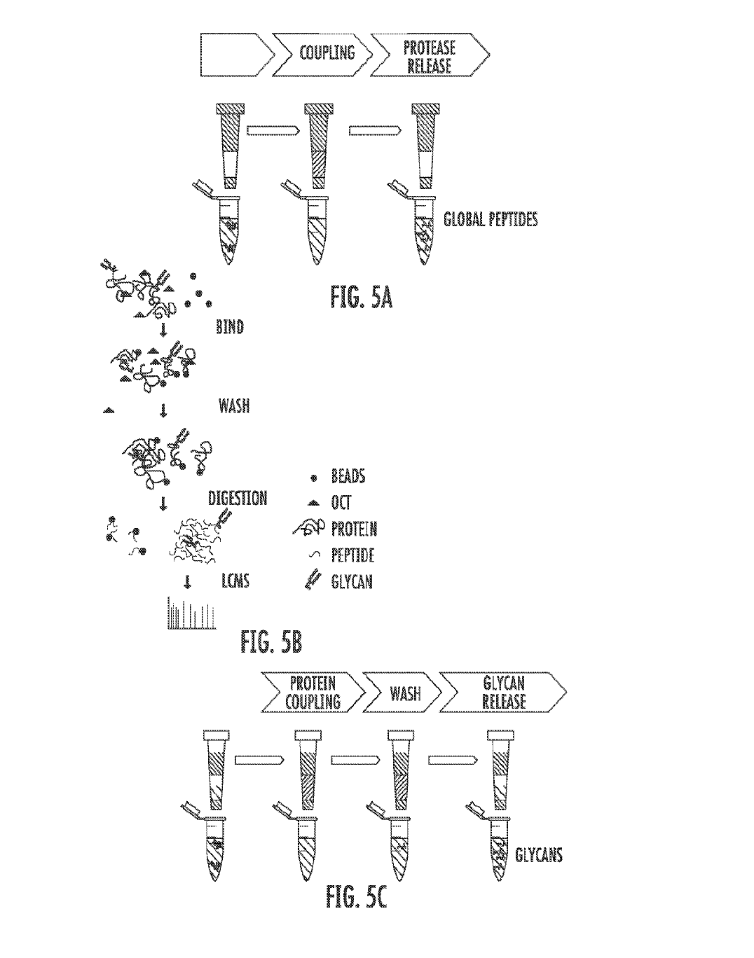

Tissue proteomics is important for the identification of disease biomarkers, treatment targets and help in the understanding of the pathological characteristics of tissues. Currently, most of the tissue proteomic studies are performed on frozen tissues or FFPE embedded tissues. Due to the malicious effect of OCT to the mass spectrometer, only a handful of proteomics studies have been performed on OCT embedded tissues (Asomugha et al., 2010, Somiari et al., 2003; Nirmalan et al., 2011; Palmer-Toy et al., 2005; Scicchitano et al., 2009). OCT embedded tissues are studied using either two-dimensional gel electrophoresis (2D DIGE) technology or shot gun proteomics using LC-MS/MS. 2D DIGE could separate proteins from OCT; however, most of the LC-MS/MS studies of OCT embedded tissue had OCT contamination resulting in fewer protein identifications (Nirmalan et al., 2011; Palmer-Toy et al., 2005; Scicchitano et al., 2009). Recently, studies demonstrated that OCT embedded tissues could be used for glycoproteomic analysis using solid-phase extraction of glycopeptide (SPEG) (Tian et al., 2011). The glycopeptides were chemically immobilized to the solid support using oxidized glycan tags when the non-glycopeptides and OCT were removed from the immobilized peptides before the enzymatic release of N-glycopeptides. To analyze global proteome of tissues, a chemical immobilization of proteins for peptide extraction was employed based on the capture of proteins using beads containing amino groups (FIG. 5). To remove OCT from the tissue sample, proteins were extracted from tissues and chemically immobilized onto the solid phase by reductive amination; however, inert OCT polymers from OCT-embedded did not get immobilized on the beads and was separated by washing the beads. Furthermore, the beads conjugated to proteins were reduced carbamidomethylated and proteolyzed to release the peptides for proteomics analysis (FIG. 5).

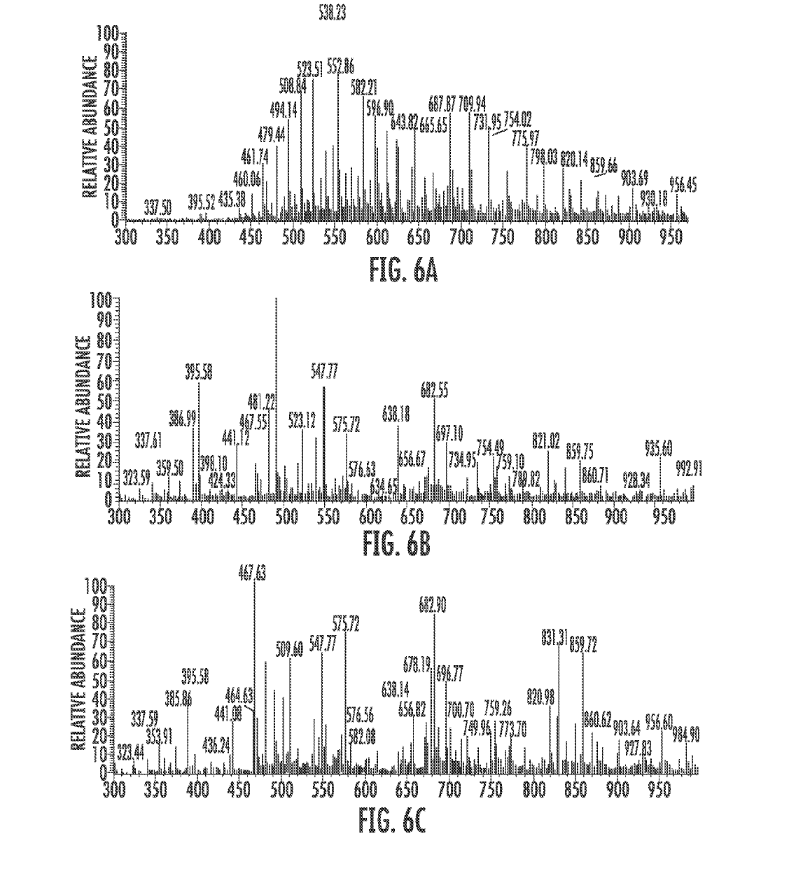

To develop a procedure to remove the OCT, Human serum albumin (HSA) with and without OCT was used as a model protein. The tryptic peptides from HSA were directly analyzed by TSQ Quantum by direct infusion ESI. FIG. 6A shows the ESI spectrum of OCT contaminated HSA digested with trypsin demonstrating a regular bell shaped curve MS pattern with mass values of 44 Da, 22 Da and 14.6 Da apart. These clearly observed peaks correspond to different charge states of polyethylene glycol presented in OCT. OCT polymer overshadows the albumin peptides. In MS, OCT dominates the mass spectrum, indicating preferential ionization of OCT compared to albumin peptides. At 20% intensity of base peak, only 11 peptide peaks out of the 45 HSA peaks were detected in OCT contaminated HSA (10% OCT in volume/HSA weight). In contrast, HSA digest in OCT had 46 unique polymer peaks that suppressed the ionization of peptides and overshadowed these peptides in the mass spectrum. To remove OCT interferences from the sample, OCT contaminated HSA was first chemically immobilized onto beads using reductive amination, beads were then washed with various conditions, and the immobilized HSA was digested using trypsin. The released peptides were analyzed using ESI-MS (FIG. 6B). After washing beads with PBS, 1.5M NaCl and water, it was found that OCT peaks completely disappeared and HSA tryptic peptide peaks were visible in the mass spectrum. None of the 46 polymer peaks uniquely observed in OCT sample was observed after CIPPE. In this embodiment of the presently disclosed method, proteins were bound to solid phase and the inert OCT polymers were washed away, resulting in the complete removal of OCT form chemically immobilized proteins. The results showed that CIPPE removed OCT contaminants from protein sample, making high throughput proteomic analysis OCT-embedded tissues using mass spectrometry possible. However, it was observed that the fingerprint of tryptic peptides of albumin was different between CIPPE and in solution digest of HSA. Only 24 out of 45 HSA peptide peaks from non-OCT HSA were detected after OCT removal using CIPPE (FIG. 6C), which may have been due to OCT embedding or the sample process using CIPPE.

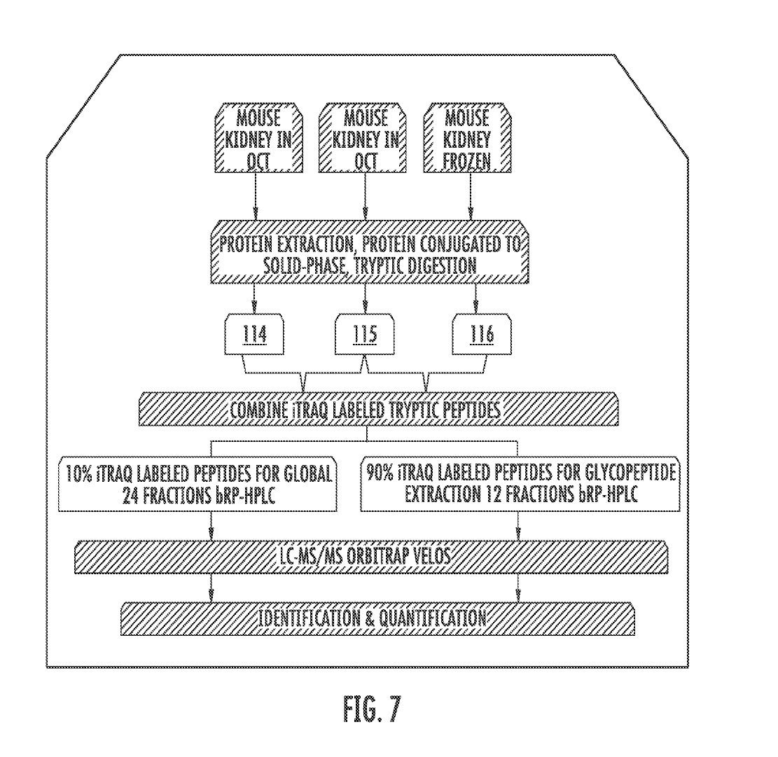

With the developed method to remove OCT contamination, the analysis of OCT embedded tissues was performed to study the impact of tissue embedding with OCT on proteomics and glycoproteomics. A complex biological tissue from mouse kidney was analyzed. Mouse kidney tissue was divided into two halves. One half was embedded in OCT and the other half was directly frozen. An OCT-embedded tissue (labeled with iTRAQ 114), a technical replicate of OCT-embedded tissue (labeled with iTRAQ115), and a frozen tissue (labeled with iTRAQ 116) were lysed and equal amount of proteins from the three tissues were used for quantitative proteomic profiling using chemical immobilization and iTRAQ methodology (FIG. 7). Proteins from each sample were first bound to beads, followed by washing. Proteins were further reduced and alkylated on beads. Finally, proteins were released from beads using proteolysis, and the released peptides were iTRAQ labeled. Samples were split into two parts, 90% of sample was used for glycoproteomic analysis and 10% of sample was used for global proteomic analysis. In global proteomic analysis, basic reverse phase was used to generate twenty-four offline fractions, and each fraction was subjected to LC-MSMS analysis using Orbitrap Velos. In glycoproteomic analysis, the sample was subjected to glycopeptide enrichment using the SPEG method. Deglycosylated peptides were then analyzed using mass spectrometry (FIG. 7).

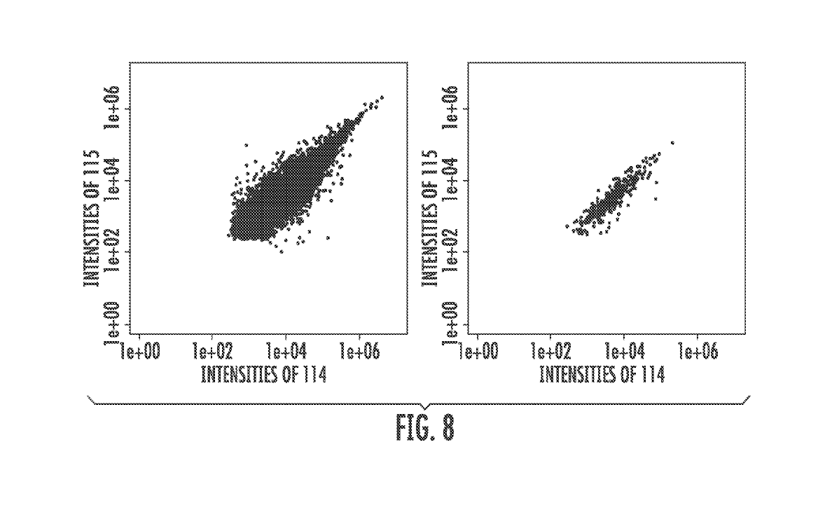

From the global proteomic analysis of iTRAQ labeled tryptic peptides, 3857 proteins were identified on the basis of at least two peptides over thresholds score of 1% FDR. Quantification results are depicted in FIG. 8. Each dot represents a peptide spectra match. The replicates 114 and 115 showed little variance and little spread in the scatter indicating high quantitative reproducibility of the method (FIG. 8A). 95% of proteins showed ratio within the interval of 0.594 to 1.821 between 114 and 115. Equal percentage of the remaining proteins (i.e. 2.5%) fell either above 1.821 or below 0.594. The correlation between 114 and 115 channel was 0.92 for global proteomic analysis. From the analysis of replicate OCT-embedded tissues using CIPPE and MS/MS, it is estimated that proteins with changes beyond ratios of 0.59 and 1.83 are considered differentially expressed with 5% FDR. Using Orbitrap Velos, 468 unique glycosylated peptides were identified. Similarly, glycopeptides showed little variance (FIG. 8B) and 95% of glycoproteins showed a ratio within the interval of 0.21 to 2.44. Correlation between channels is 0.91 (FIG. 8B). These results indicated good analytical replications for global and glycoproteomics of OCT-embedded tissues using chemical immobilization. The results showed that accurate quantitation could be achieved on OCT embedded tissue using chemical immobilization, iTRAQ labeling, and tandem mass spectrometry.

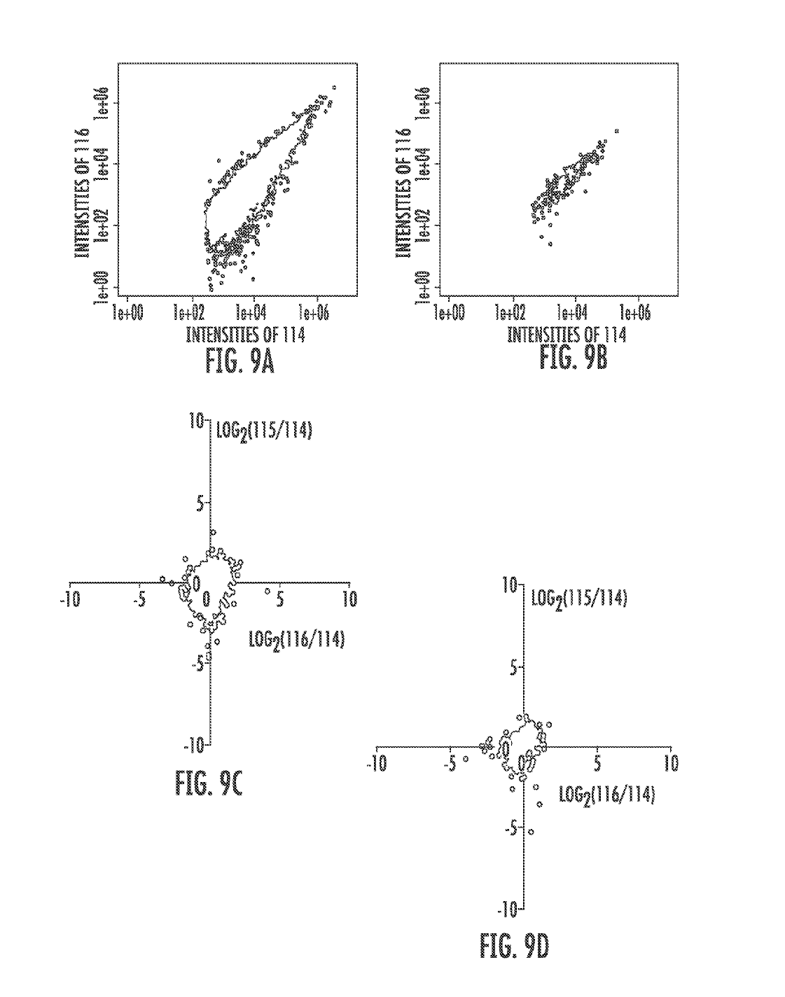

The scatter plot of intensities of two channels 116 and 114 (frozen tissue/OCT embedded tissue) showed similar patterns as the technical replicates of OCT-embedded tissues (FIGS. 8A and 9A). The quantitative distribution are roughly symmetrical only with little spread from 1:1 line in the scatter plot, indicating high quantitative similarity between frozen and OCT-embedded tissue (FIG. 9A). A vast majority of the proteins belonged to 1:1 ratio irrespective of the intensity of iTRAQ channel and 86.36% of the proteins showed ratio within the interval of 0.59 to 1.83 (the same cut off from the replicate analysis). A percentage of 2.22 proteins showed a ratio above 1.821 while 11.41% of proteins displayed a ratio below 0.59. This percentage of down-regulated proteins indicated that there were apparently more peptides extracted from OCT embedded tissue compared to the frozen tissue. The correlation between 114 and 116 is 0.92 indicating good similarity from quantitation perspective for frozen and OCT-embedded tissues. Next, differential quantification of glycoproteome related to OCT embedded and frozen tissues was investigated. FIG. 9B shows the scatter plot of frozen tissues and OCT-embedded tissues for of the identified glycopeptides. The percentage of glycoproteins having a ratio between 0.21 and 2.44 (the same cut off from the replicate analysis) was 94.82%. Similar to quantitative analysis of global proteome, the quantitative distribution glycopeptide shows little variance indicating high quantitative similarity of glycoproteome between frozen and OCT embedded tissues (FIG. 9B). The correlation between 114 and 116 is 0.90, similar to replicate analysis of OCT-embedded tissues. To determine whether there were significant differences between OCT-embedded and frozen tissues, log₂(116/114) in X axis and log₂(115/114) in Y axis was plotted for global proteomics (FIG. 9C) and glycoproteomics (FIG. 9D). All proteins and glycoproteins are close to the origin. The results demonstrate that quantitative analysis of OCT embedded tissue is feasible. It has been shown that CIPPE is a method for quantitative analysis of protein expression and protein glycosylation in tissue proteomics from frozen and OCT-embedded tissues. Using this method, thousands of proteins from OCT-embedded tissues have been successfully identified. CIPPE has potential to be used for other PTM analysis like phosphorylation, ubiquitation and acetylation.

In addition to the removal of OCT from OCT-embedded tissues, this method could be used to extract proteins from tissues for tissue proteomics. Compared to the proteins from body fluids, the proteins from tissues are more difficult to extract in order to obtain a complete proteome due to the three-dimensional structures of tissues and solubility of certain tissue proteins. During the proteomic analysis of tissues, detergents such as sodium dodecyl sulfate (SDS), NP-40, or Triton X-100, are often used for protein extraction to solubilize the membrane proteins from tissues. However, detergents also distort mass spectrometric detection of peptides, similar to the observed spectra from OCT-contaminated HSA (FIG. 6A). In addition, these detergents, similar to OCT, bind to a reverse phase column, commonly used online with a mass spectrometer, further impairing the capability of tissue proteomics using LC-MS-MS/MS. CIPPE method is not only able to remove high concentration OCT, but also the detergents from the tissues samples introduced during the protein extraction for proteomics analysis.

In some cases, there is incomplete release of all tryptic peptides after the proteins are chemically immobilized onto the beads and peptides are released from beads using trypsin digestion. For protein identification and quantification, it is not necessary to recover all tryptic peptides. In the situations where all tryptic peptides are needed for the proteomic analysis, a cleavable linker to the solid phase could be used to capture and release all peptides.

This study shows that tissues embedded in OCT can be analyzed using shotgun proteomics. The CIPPE methodology described here was used to conduct global and glycoproteomics analyses of tissues embedded in OCT. When adopted, this protocol is highly efficient in the removal of contaminants Data indicated that OCT does not seem to impact the tissue proteome and glycoproteome. Therefore, CIPPE can be used for the analysis of OCT embedded tissue for proteomics and PTMs analysis like glycosylation, leading to the possibility of the discovery of potential biomarkers.

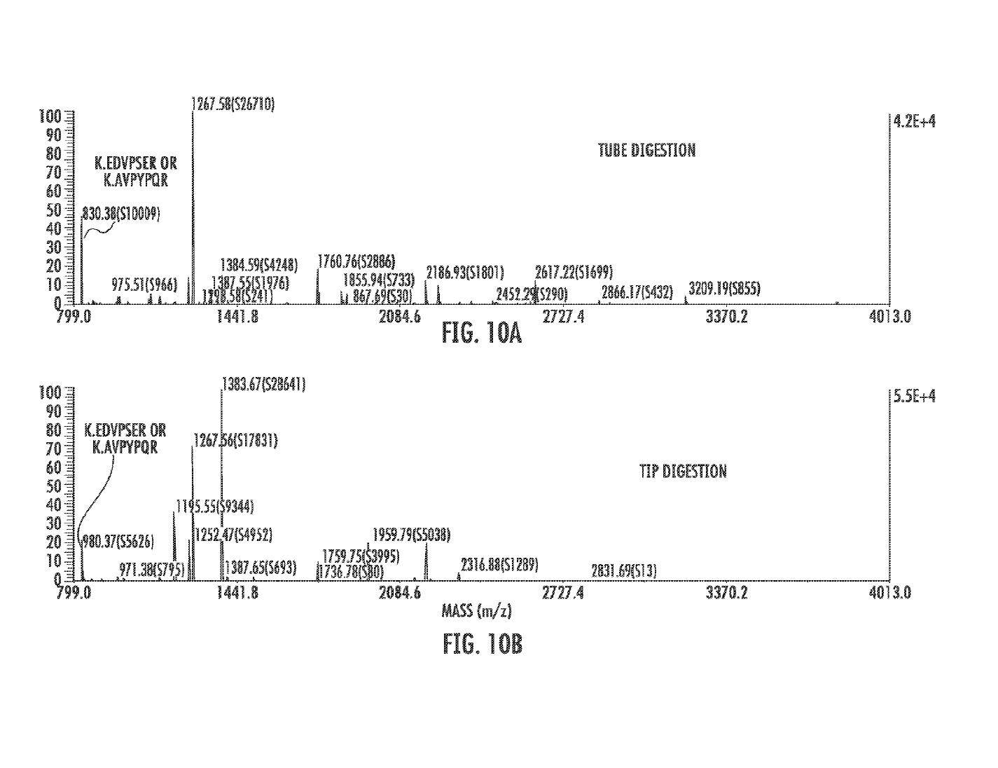

FIGS. 10-10B show representative MALDI spectra of released tryptic global peptides released from casein immobilized to solid phase by reductive amination with a mass range of 500-4000 using an embodiment of the tube digestion method and the tip method. K.AVPYPQR (SEQ ID NO:355) is a peptide from beta casein.

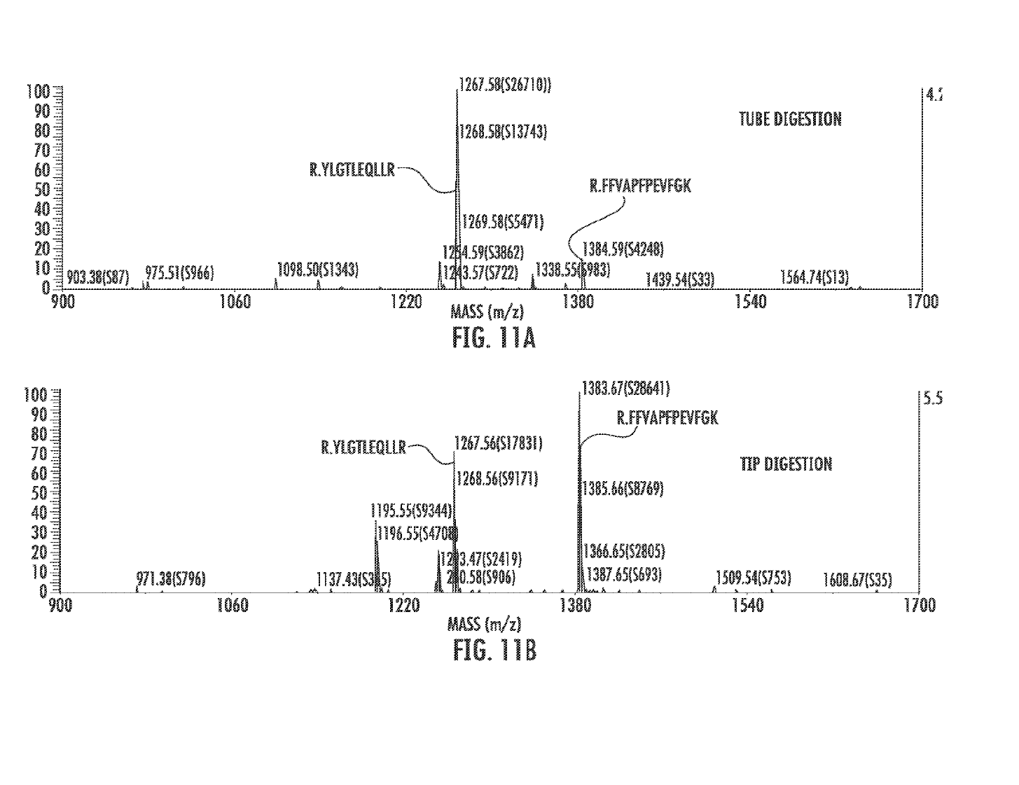

FIGS. 11A-11B show representative MALDI spectra of released tryptic peptides from casein immobilized to the solid phase in a tip with a mass range of 900-1700 using an embodiment of the tube digestion method and the tip method. R.FFVAPFPEVFGK (SEQ ID NO:357) and R.YLGYLEQLLR (SEQ ID NO:358) are peptides from alpha-S1-casein.

### Example 3

### High Throughput Analysis of N-Glycans Using Glycoprotein Immobilization for Glycan Extraction with Aldehyde Tips

### Introduction

Aberrant glycosylation plays a critical role in many diseases where disease-associated glycans may be discovered for diagnosis and treatment.

To analyze N-glycans, a robust method for isolation of N-glyans using glycoprotein immobilization for glycan extraction (GIG) has been recently developed (Yang et al., 2013; Shah et al., 2013). Meanwhile, tip columns in combination with a robotic liquid handling system has shown its potential in high throughput sample processing for mass spectrometry analysis (Chen and Zhang, 2013).

To facilitate high throughput N-glycan analysis, a novel aldehyde tip was devised and tested for its performances on extracting N-glycans from human serum with a robotic liquid handling unit.

The incubation time for each of the major steps of N-glycan isolation was optimized, multiple parallel isolations of glycans were performed, the N-glycans extracted were analyzed by mass spectrometry and the reproducibility was assessed.

### Methods

Preparation of Aldehyde Tip:

A round frit (2-mm-diameter and 1-mm-thick, pore size 15-45 microns) were first pushed into the pipette tip end (Disposable Automation Research Tips, Thermo Fisher Scientific, Waltham, Mass.). Two hundred microliters of aldehyde resin (50% slurry) was then loaded into each pipette tip. Liquids were blown out of the tip and a 5 mm round frit was pushed into the tip to secure the aldehyde resin between the two frits. Each tip was washed 5 times with 200 μL of water and conditioned 5 times with coupling buffer (100 mM sodium carbonate, pH=10) by aspirating and dispensing the solution.

Isolation of N-Glycans:

For protein immobilization, each tip was pipetted up and down in protein sample in sodium carbonate buffer (pH=10), followed by sodium cyanoborohydride containing PBS buffer (pH=7.4), and then Tris blocking buffer (pH=7.6). Sialic acid modification was performed by pipetting each tip up and down in p-toluidine solution (pH=4-6). For the washing step, each tip was pipetted up and down in 6 mL of 1% formic acid, 6 mL of 1M NaCl, 6 mL of 10% acetonitrile, and finally 6 mL of water. N-Glycan release occurred by pipetting each tip through 5 mM ammonium bicarbonate solution (pH=7.5) containing 2 μL PNGase F. The released N-glycans in the supernatant were collected and dried in vacuum. The extracted N-glycans were resuspended in HPLC grade water.

MALDI-MS Analysis:

N-glycans were analyzed using Axima MALDI Resonance mass spectrometer (Axima, Shimadzu, Columbia, Md.). Four microliters of dimethylamine (DMA) were mixed with 200 μL of 2,5-dihydrobenzoic acid (DHB) (100 μg/μL in 50% acetonitrile, 0.1 mM NaCl) as matrix-assisted laser desorption ionization (MALDI) matrix. Maltoheptaose (DP7) was spiked into each sample as a glycan standard at 25 mM. The laser power was set to 100 for two shots each in 100 locations per spot. The average MS spectra (200 profiles) were used for glycan assignment by comparing to the database of glycans previously analyzed by MALDI tandem mass spectrometry (MALDI-TOF-MS/MS). The assigned glycans were confirmed from human serum established in the literature.

### Results

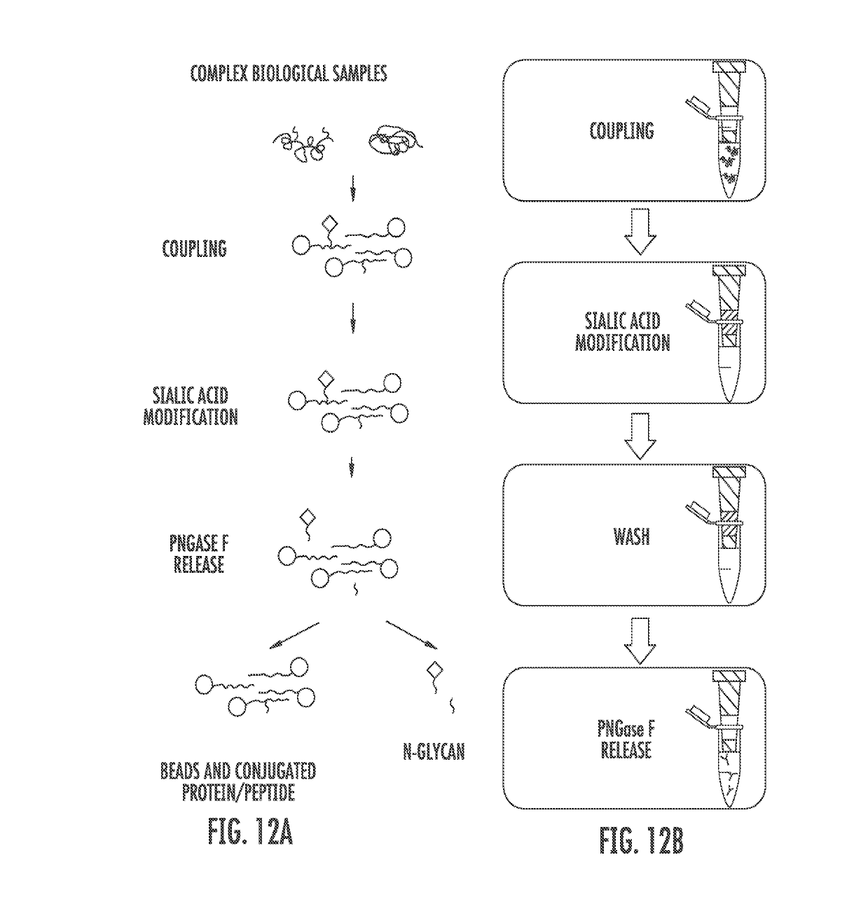

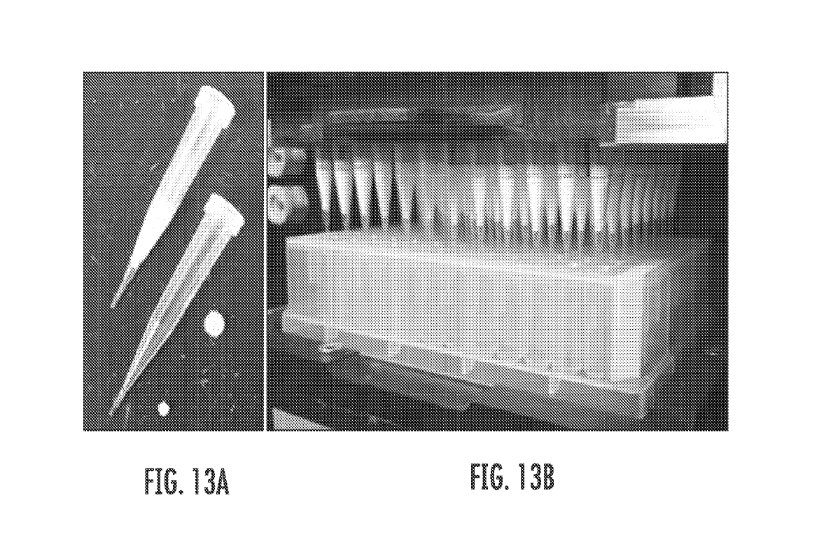

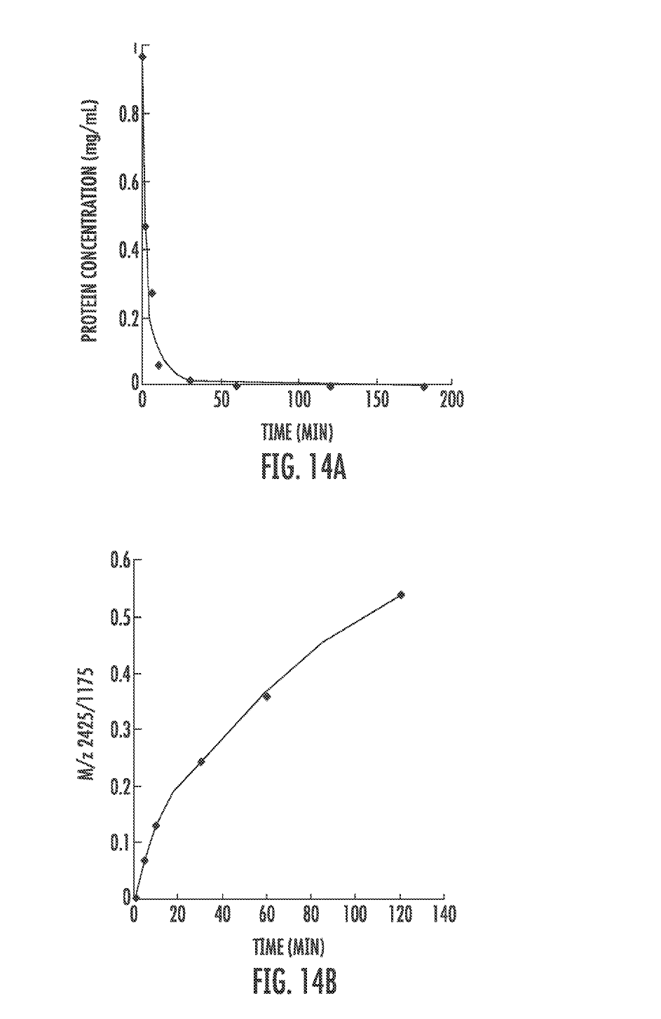

FIGS. 12A-12B show an embodiment of a workflow scheme of N-glycan isolation. Proteins from samples were first immobilized onto beads/tip columns, sialic acid was then modified with p-toluidine, the beads/tips were subsequently washed extensively in 1% formic acid, 1M NaCl, 10% acetonitrile, and water, and the N-glycans were finally released with PNGase F. Photographs of an unpacked and packed aldehyde tip (FIG. 13A) and 96-well aldehyde tips loaded in a robotic liquid handling system for automated glycan extraction (FIG. 13B) are also shown. The reaction time for coupling and PNGase F release was optimized. Serum proteins were slowly pipetted through aldehyde tips for various amount of time and complete coupling was achieved after 30 min reaction (FIG. 14A). After extensive washing and sialic acid labeling, the N-glycans from serum proteins were released from the aldehyde tips with PNGase F for various times. N-glycan was still releasing after 2 hours (FIG. 14B).

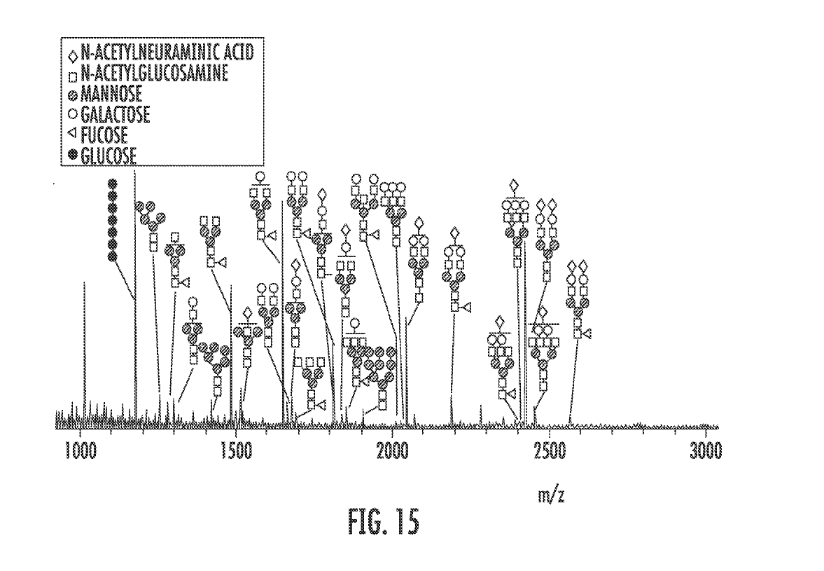

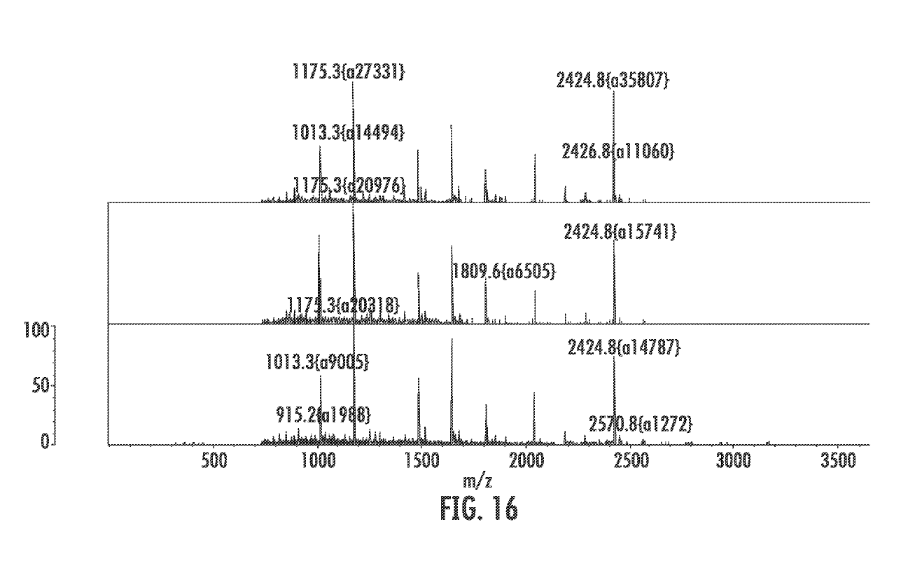

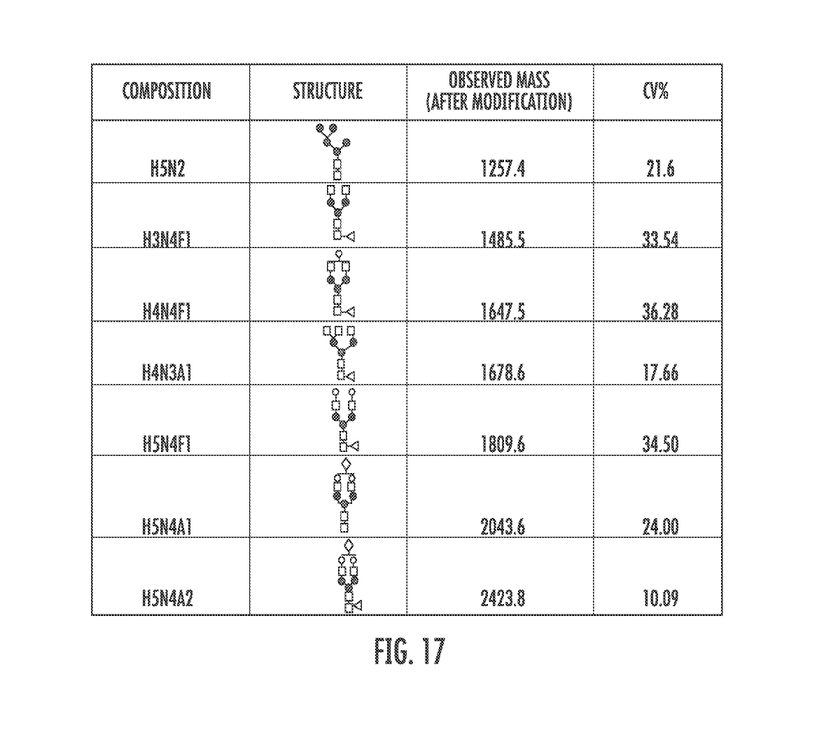

MALDI-MS profiles of serum N-glycans isolated with the aldehyde tips were generated (FIG. 15). FIG. 16 shows representative MALDI profiles of three isolations of N-glycan from human serum. The glycans from the three isolations were quantified and the reproducibility of N-glycan isolation was assessed (FIG. 17).

It was found that the application of aldehyde tips significantly reduced the processing time of N-glycan isolation and that aldehyde tips have great potential in achieving automation of N-glycan isolation for high throughput sample preparation when used in combination with liquid handling robotic systems.

### Example 4

### Solid Phase Labeling of Glycans and Proteins for Quantitative Glycopeptide Analysis Introduction

Glycosylation is one of the most abundant post-translational modifications on proteins. Sialic acids on glycoprotein are typically found at the terminal residue of glycans. Sialic acids play crucial role in cell surface interactions, protect cells from membrane proteolysis, help in cell adhesion, and determine half-life of glycoprotein in blood. The degree of sialylation has been demonstrated to be a consequence of diseases.

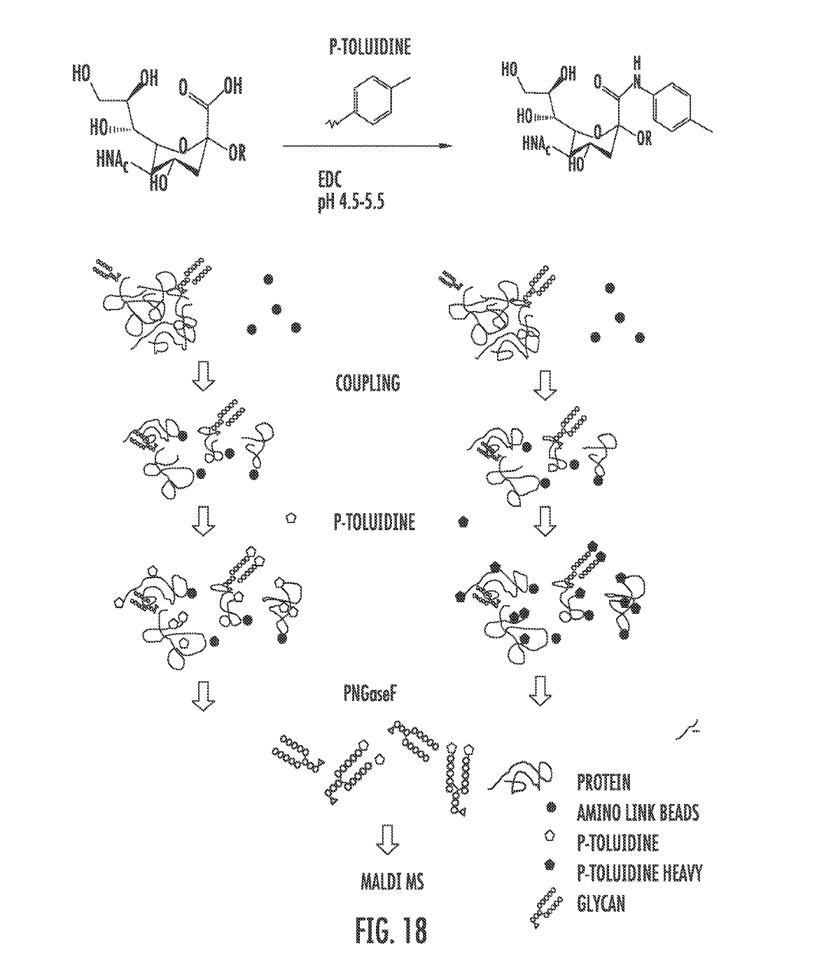

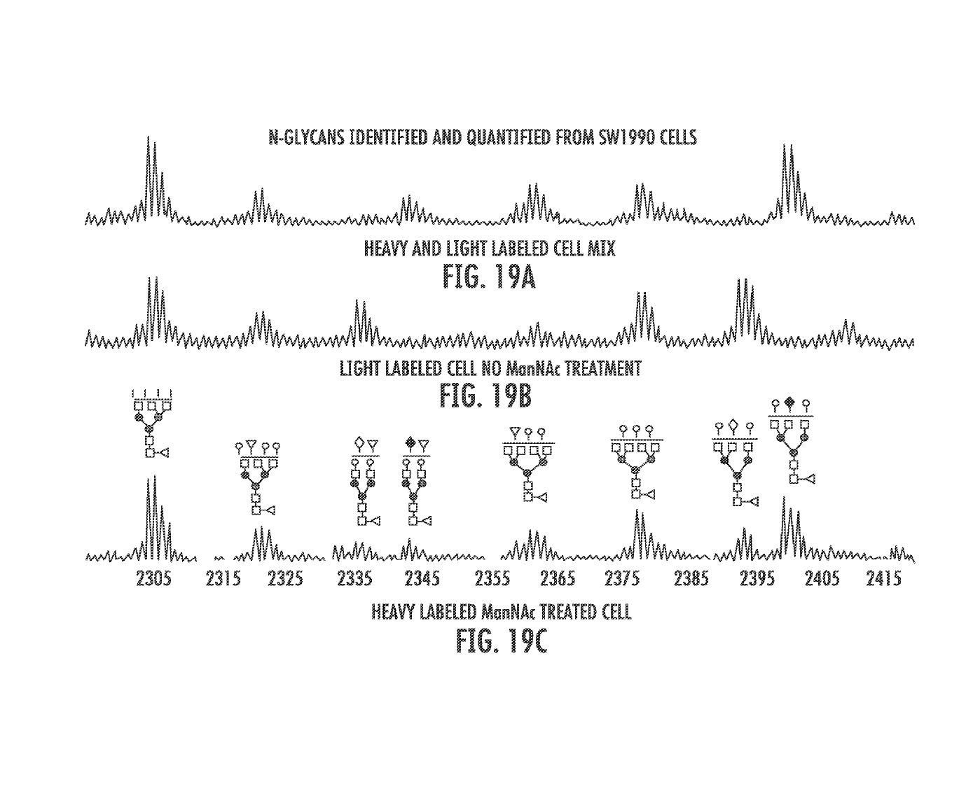

A strategy has been developed to label aspartic acid, glutamic acid and sialylated glycans with stable isotopic tags in a single process for quantitative MS analysis. A quantitative method of solid-phase sialic acid labeling is described (FIG. 18). N-glycans were identified and quantified from SW1990 cells (FIGS. 19A-19C; SW1990 Cells with and without 1,3,4-O-Bu3ManNAc treatment). 87 N-glycans and 32 sialylated N-glycans were identified and 14 sialylated N-glycans were relatively quantified (Table 6).

Advantages of labeling include stabilization of the sialylated glycan and removal of the negative charge from N-glycans; the sample is first bound to the beads and hence the proteins after removal of N glycans can be analyzed using tryptic digestion; and along with sialic acid, aspartic acid and glutamic acid get modified and can be used for peptide/protein quantitation.

TABLE 6
Sialylated N-glycans

| | | | | Sialic | [M + | | |
|---|---|---|---|---|---|---|---|
| Core + Na | Fucose | HexNAC | Hexose | Acid | Na]+ | H/L | Stdev |
| Core + Na | 0 | 2 | 2 | 1 | 2043.89 | 1.29 | 0.44 |
| Core + Na | 1 | 2 | 2 | 1 | 2189.95 | 1.09 | 0.17 |
| Core + Na | 2 | 2 | 2 | 1 | 2336.01 | 1.10 | 0.13 |
| Core + Na | 1 | 3 | 2 | 1 | 2393.04 | 1.95 | 0.11 |
| Core + Na | 2 | 3 | 2 | 1 | 2539.10 | 1.31 | 0.10 |
| Core + Na | 1 | 3 | 3 | 1 | 2555.10 | 1.31 | 0.45 |
| Core + Na | 1 | 2 | 2 | 2 | 2570.21 | 1.02 | 0.13 |
| Core + Na | 3 | 3 | 2 | 1 | 2685.16 | 1.00 | 0.17 |
| Core + Na | 2 | 3 | 3 | 1 | 2701.15 | 1.03 | 0.23 |
| Core + Na | 1 | 4 | 3 | 1 | 2758.19 | 1.66 | 0.31 |
| Core + Na | 1 | 3 | 2 | 2 | 2773.31 | 1.70 | 0.18 |
| Core + Na | 2 | 4 | 3 | 1 | 2904.25 | 2.04 | 0.50 |
| Core + Na | 1 | 3 | 3 | 2 | 2935.36 | 0.98 | 0.21 |
| Core + Na | 3 | 4 | 3 | 1 | 3050.31 | 1.39 | 0.30 |

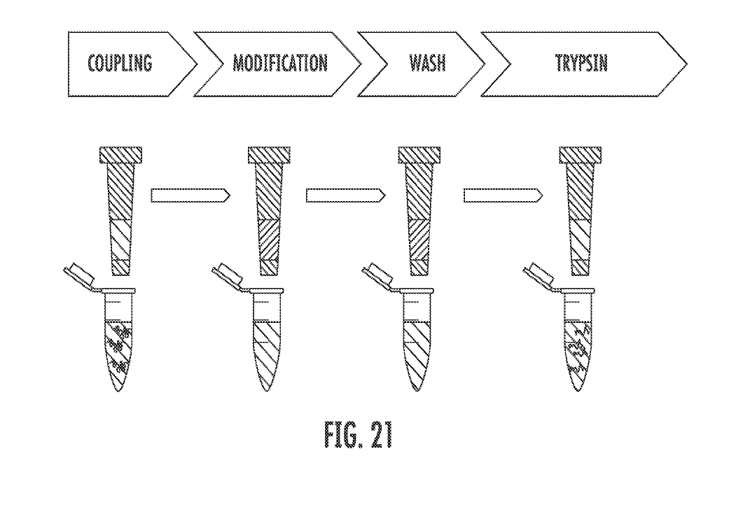

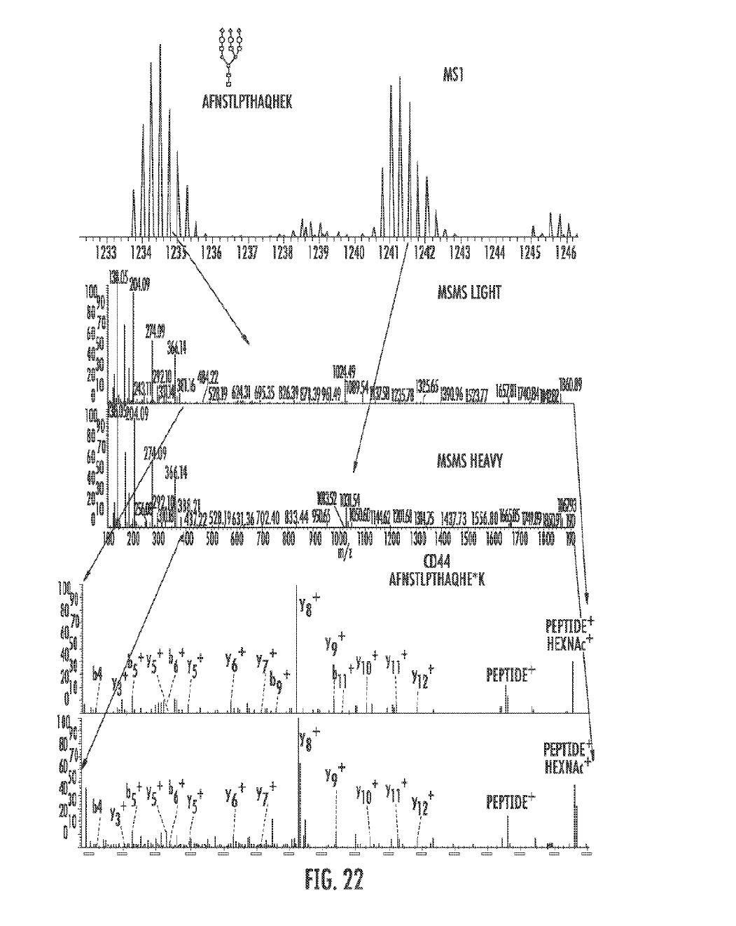

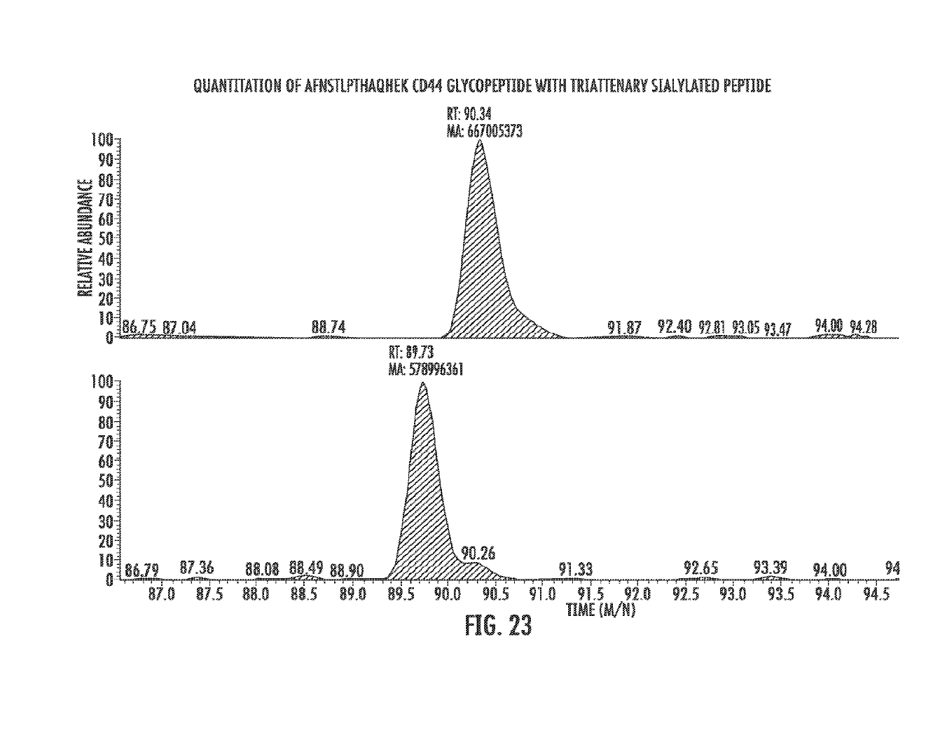

Glycopeptide analysis was performed using basic reverse phase fractionation (FIG. 21). Sample preparation including labeling was automated using liquid handling robotic systems (FIG. 22). Results showed quantitation of AFNSTLPTHAQHEK (SEQ ID NO: 354) CD44 glycopeptide with triattenary sialylated peptide (FIGS. 22-23).

TABLE 7
Results from Glycopeptide Analysis

| | Number of |
|---|---|
| Sample | samples |
| MSMS containing oxonium ions | 4069 |
| MSMS containing sialylated | 547 |
| oxonium ions | |
| Unique sialylated oxonium | 390 |
| ions precursor | |
| Global proteins identified | 2681 |
| Proteins down regulated due | 116 |
| to ManNAc treatment | |
| Proteins upregulated due | 243 |
| to ManNAc treatment | |

In summary, a comprehensive quantitative N-glycosylation analysis was performed using stable isotope labeling on both glycans and proteins (glycosite-containing peptide, glycans, and glycopeptides). 1,3,4-O-Bu3ManNAc resulted in an increase in sialylation at specific glycosites.

### Example 5

### Discussion

In some embodiments, the presently disclosed subject matter provides a pipette tip comprising a chemical moiety. In other embodiments, the presently disclosed subject matter provides a hydrazide bead packed pipette tip for rapid, reproducible, and automated N-linked glycopeptide isolations. Using bovine fetuin as a standard glycoprotein, the incubation time was determined for each major step of glycopeptide isolation. Using commercially available human serum, multiple parallel isolations of glycopeptides were performed using hydrazide tips with a liquid handling robotic system. It was determined that with the hydrazide tip, the processing time was significantly decreased from the original three to four day SPEG manual procedure to less than an eight hour automated process. In addition, it was demonstrated that the hydrazide tip could perform glycopeptide isolations in a reproducible manner. The hydrazide tip was compatible with liquid handling robotics and has great potential in the automation of glycopeptide isolations for high throughput sample preparation.

In addition, to facilitate high throughput N-glycan analysis, a novel aldehyde tip was devised and successfully extracted N-glycans from human serum with a robotic liquid handling unit.

Further, a quantitative method of solid-phase sialic acid labeling was described. p-toluidine was successfully used to modify the acid component of proteins and sialylated glycans with a reliable and robust method for quantitation of glycan and glycopeptide.

The presently disclosed methods have been shown herein to be useful for a variety of glycoproteins or polypeptides.

### REFERENCES

All publications, patent applications, patents, and other references mentioned in the specification are indicative of the level of those skilled in the art to which the presently disclosed subject matter pertains. All publications, patent applications, patents, and other references, including Appendix A which is attached hereto, are herein incorporated by reference to the same extent as if each individual publication, patent application, patent, and other reference was specifically and individually indicated to be incorporated by reference. It will be understood that, although a number of patent applications, patents, and other references are referred to herein, such reference does not constitute an admission that any of these documents forms part of the common general knowledge in the art.
Ahram, M.; Flaig, M. J.; Gillespie, J. W.; Duray, P. H.; Linehan, W. M.; Ornstein, D. K.; Niu, S.; Zhao, Y.; Petricoin, E. F.; Emmert-Buck, M. R., Proteomics 2003, 3 (4): 413-421.
Asomugha, C.; Gupta, R.; Srivastava, O., Molecular Vision 2010, 16: 476.
Bantscheff, M.; Lemeer, S.; Savitski, M. M.; Kuster, B., Analytical and Bioanalytical Chemistry 2012, 1-27.
Boersema, P. J.; Geiger, T.; Wisniewski, J. R.; Mann, M., Molecular and Cellular Proteomics: MCP 2013, 12:158-71.
Chaurand, P.; Schwartz, S. A.; Caprioli, R. M., Analytical Chemistry 2004, 76(5):86-93.
Chen J, Shah PK, and Zhang H. Analytical Chemistry 2013, 85(22):10670-4.
Conway, J.; Sloane, N.; Bannai, E., Sphere packings, lattices, and groups. Springer: 1999; p 703.
Dirksen, A.; Yegneswaran, S.; Dawson, P. E., Angew. Chem. Int. Ed. Engl. 2010, 49: 2023-7.
Li, Q. K.; Shah, P.; Li, Y.; Aiyetan, P. O.; Chen, J.; Yung, R.; Molena, D.; Gabrielson, E.; Askin, F.; Chan, D. W.; Zhang, H., Journal of Proteome Research 2013, 12(8):3689-96.
Li, Y.; Sokoll, L. J.; Barker, P. E.; Zhang, H.; Chan, D. W., Clinical Proteomics 2008, 4 (1):58-66.
Li, Y.; Tian, Y.; Rezai, T.; Prakash, A.; Lopez, M. F.; Chan, D. W.; Zhang, H., Analytical Chemistry 2011, 83:240-5.
Liu, H.; Sadygov, R. G.; Yates, J. R., 3rd, Analytical Chemistry 2004, 76: 4193-201.
Magdeldin, S.; Yamamoto, T., Proteomics 2012, 12(7): 1045-1058.
Maggino, T.; Gadducci, A., European journal of Gynaecological Oncology 2000, 21:64-9.
Nirmalan, N. J.; Hughes, C.; Peng, J.; McKenna, T.; Langridge, J.; Cairns, D. A.; Harnden, P.; Selby, P. J.; Banks, R. E., Journal of Proteome Research 2011, 10(2):896.
Palmer-Toy, D. E.; Krastins, B.; Sarracino, D. A.; Nadol Jr, J. B.; Merchant, S. N., Journal of Proteome Research 2005, 4(6):2404-2411.
Ralton, L. D.; Murray, G. I., Journal of Clinical Pathology 2011, 64(4):297-302.
Sanda, M.; Pompach, P.; Brnakova, Z.; Wu, J.; Makambi, K.; Goldman, R., Molecular and Cellular Proteomics: MCP 2013, 12:1294-305.
Schroder, F. H.; Habbema, D. F.; Roobol, M. J.; Bangma, C. H., European Urology 2007, 51:588-90.

Scicchitano, M. S.; Dalmas, D. A.; Boyce, R. W.; Thomas, H. C.; Frazier, K. S., Journal of Histochemistry and Cytochemistry 2009, 57(9):849-860.
Setou, M., Imaging Mass Spectrometry: Protocols for Mass Microscopy. Springer, 2010.
Shah, P. et al., Analytical Chemistry 2013, 85:3606-3613.
Sia, S. K.; Whitesides, G. M., Electrophoresis 2003, 24:3563-76.
Somiari, R. I.; Sullivan, A.; Russell, S.; Somiari, S.; Hu, H.; Jordan, R.; George, A.; Katenhusen, R.; Buchowiecka, A.; Arciero, C., Proteomics 2003, 3(10): 1863-1873.
Tian, Y.; Bova, G. S.; Zhang, H., Analytical Chemistry 2011, 83(18): 7013-7019.
Tian, Y.; Esteva, F. J.; Song, J.; Zhang, H., Molecular and Cellular Proteomics: MCP 2012, 11(6):M111.011403.

Tian, Y.; Gurley, K.; Meany, D. L.; Kemp, C. J.; Zhang, H., Journal of Proteome Research 2009, 8(4):1657-1662.
Tian, Y.; Kelly-Spratt, K. S.; Kemp, C. J.; Zhang, H., Clinical Proteomics 2008, 4(3): 117-136.
Tian, Y.; Kelly-Spratt, K. S.; Kemp, C. J.; Zhang, H., Journal of Proteome Research 2010, 9(11):5837-5847.
Tian, Y.; Zhou, Y.; Elliott, S.; Aebersold, R.; Zhang, H., Nature Protocols 2007, 2 (2):334-339.
Turbett, G. R.; Sellner, L. N., Diagnostic Molecular Pathology: the American Journal of Surgical Pathology, Part B 1997, 6(5):298.
Vincenti, D. C.; Murray, G. I., Clinical Biochemistry 2013, 46(6):546-51.
Wang, X.; Zhang, A.; Wang, P.; Sun, H.; Wu, G.; Sun, W.; Lv, H.; Jiao, G.; Xu, H.; Yuan, Y.; Liu, L.; Zou, D.; Wu, Z.; Han, Y.; Yan, G.; Dong, W.; Wu, F.;
Dong, T.; Yu, Y.; Zhang, S.; Wu, X.; Tong, X.; Meng, X., Molecular and Cellular Proteomics: MCP 2013, 12:1226-38.
Wu, J.; Xie, X.; Nie, S.; Buckanovich, R. J.; Lubman, D. M., Journal of Proteome Research 2013, 12:3342-52.
Yang S, Li Y, Shah PK, and Zhang H. Analytical Chemistry 2013 85:5555-5561.
Zeng, Y.; Ramya, T. N.; Dirksen, A.; Dawson, P. E.; Paulson, J. C., Nature Methods 2009, 6, 207-9.
Zhang, H.; Chan, D. W., Cancer Epidemiology Biomarkers and Prevention 2007, 16, (10), 1915-1917.
Zhang, A. H.; Sun, H.; Yan, G. L.; Han, Y.; Wang, X. J., Applied Biochemistry and Biotechnology 2013, 170:774-86.
Zhang, H.; Li, X. J.; Martin, D. B.; Aebersold, R., Nature Biotechnology 2003, 21:660-6.
Zhang, H.; Loriaux, P.; Eng, J.; Campbell, D.; Keller, A.; Moss, P.; Bonneau, R.; Zhang, N.; Zhou, Y.; Wollscheid, B.; Cooke, K.; Yi, E. C.; Lee, H.; Peskind, E. R.; Zhang, J.; Smith, R. D.; Aebersold, R., Genome Biology 2006, 7:R73.
Zhang, H.; Yi, E. C.; Li, X. J.; Mallick, P.; Kelly-Spratt, K. S.; Masselon, C. D.; Camp, D. G., 2nd; Smith, R. D.; Kemp, C. J.; Aebersold, R., Molecular and Cellular Proteomics: MCP 2005, 4:144-55.
Zhang, H.; Chan, D. W., Cancer Epidemiology Biomarkers and Prevention 2007, 16(10):1915-1917.
Zhou, Y.; Aebersold, R.; Zhang, H., Analytical Chemistry 2007, 79:5826-37.

Although the foregoing subject matter has been described in some detail by way of illustration and example for purposes of clarity of understanding, it will be understood by those skilled in the art that certain changes and modifications can be practiced within the scope of the appended claims.

That which is claimed:

1. A pipette tip comprising an elongate body having a proximal end adapted to connect to and be in fluid communication with an outlet of a fluid dispensing device and a distal end having an opening adapted to dispense a fluid, the elongate body further comprising a fluid path between the proximal end and the distal end, wherein the fluid path comprises:
(a) a first frit proximate the distal end and a second frit proximate the proximal end, and wherein the fluid path comprises a solid phase disposed between the first frit and the second frit, the solid phase comprising:
(i) a chemical moiety capable of conjugating one or more glycoproteins through one or more oxidized glycans; or
(ii) an amino-reactive moiety capable of conjugating one or more amino groups of one or more proteins disposed in the fluid path between the first frit and the second frit; or
(iii) other chemical moieties capable of conjugating to one or more reactive groups of amino acid side chains or protein modifications disposed in the fluid path between the first frit and the second frit; or
(b) a monolith-bonded aldehyde-reactive chemical moiety, a monolith-bonded amino-reactive moiety or other chemical moieties capable of conjugating to one or more reactive groups of amino acid side chains or protein modifications.

2. The pipette tip of claim 1, wherein the chemical moiety is selected from the group consisting of one or more hydrazide beads and a hydrazide resin.

3. The pipette tip of claim 2, wherein the hydrazide resin has a particle size ranging from about 40 micrometers to about 60 micrometers.

4. The pipette tip of claim 1, wherein the pipette tip further comprises more than two frits.

5. The pipette tip of claim 1, wherein the first frit and the second frit have a pore size ranging from about 15 microns to about 45 microns.

6. A method for preparing a pipette tip, the method comprising:
(a) providing a pipette tip comprising an elongate body having a proximal end adapted to connect to and be in fluid communication with an outlet of a fluid dispensing device and a distal end having an opening adapted to dispense a fluid; and
(b) forming a fluid path between the proximal end and the distal end by one of:
(i) disposing a first frit proximate the distal end of the pipette tip and disposing thereon a solid phase comprising one of a chemical moiety capable of conjugating one or more glycoproteins through one or more oxidized glycans or an amino-reactive moiety or other chemical moieties capable of conjugating to one or more reactive groups of amino acid side chains or protein modifications capable of conjugating one or more amino groups of one or more proteins, and disposing a second frit proximate the proximal end of the pipette tip; or
(ii) disposing a monolith-bonded aldehyde-reactive chemical moiety or a monolith-bonded amino-reactive moiety or other chemical moieties capable of conjugating to one or more reactive groups of amino acid side chains or protein modifications between the distal end and the proximal end of the pipette tip.

7. The method of claim 6, wherein the chemical moiety comprises a aldehyde-reactive chemical moiety.

8. The method of claim 6, wherein the first frit and the second frit have a pore size ranging from about 15 to about 45 microns.

9. The method of claim 6, further comprising washing the solid phase after the solid phase is disposed on the first frit.

10. The method of claim 9, further comprising washing the solid phase with a liquid selected from the group consisting of water and a buffer.

11. A kit comprising at least one pipette tip of claim 1, wherein the kit further comprises a set of instructions for using the at least one pipette tip to isolate a biological molecule.

12. A high throughput method for identifying a protein, glycoprotein, or a glycan in a plurality of samples, the method comprising:
(a) providing a plurality of samples comprising at least one protein comprising at least one peptide amino group or at least one glycoprotein comprising at least one oxidized glycan or at least one reactive groups of amino acid side chains or protein modifications;
(b) disposing the plurality of samples in a plurality of pipette tips, wherein each pipette tip comprises an elongate body having a proximal end adapted to connect to and be in fluid communication with an outlet of a fluid dispensing device and a distal end having an opening adapted to dispense a fluid, the elongate body further comprising a fluid path between the proximal end and the distal end, wherein the fluid path comprises:
(i) a first frit proximate the distal end and a second frit proximate the proximal end, and wherein the fluid path comprises a solid phase disposed between the first frit and the second frit, the solid phase comprising a chemical moiety capable of conjugating one or more glycoproteins through one or more oxidized glycans or an amino-reactive moiety capable of conjugating one or more amino groups or other chemical moieties capable of conjugating to one or more reactive groups of amino acid side chains or protein modifications of one or more proteins disposed in the fluid path between the first frit and the second frit; or
(ii) a monolith-bonded aldehyde-reactive chemical moiety or a monolith-bonded amino-reactive moiety or other chemical moieties capable of conjugating to one or more reactive groups of amino acid side chains or protein modifications;
(c) conjugating the at least one protein or at least one glycoprotein comprising the plurality of samples to the solid phase chemical moieties capable of conjugating to one or more reactive groups of amino acid side chains or protein modifications or the monolith-bonded aldehyde-reactive chemical moiety or amino-reactive moiety or other chemical moieties capable of conjugating to one or more reactive groups of amino acid side chains or protein modifications;
(d) cleaving the at least one protein thereby releasing at least one peptide fragment or releasing the at least one former glycopeptide fragment or glycan; and
(e) analyzing the at least one peptide, glycan or the at least one former glycopeptide fragment to identify the protein, glycan from which the at least one peptide and glycan fragment was derived or to identify the glycoprotein from which the former glycopeptide fragment was derived; and
wherein at least one step of the method is automated.

13. The high throughput method of claim 12, wherein cleaving the at least one glycoprotein comprising at least one oxidized glycan occurs by enzymatic reaction if the at least one oxidized glycan is an N-glycan or by chemical reaction if the at least one oxidized glycan is an O-glycan.

14. The high throughput method of claim 12, wherein the cleaving of the at least one protein occurs by using a protease or a chemical.

15. The high throughput method of claim 12, wherein the cleaving of the at least one protein leaves at least one peptide, former glycopeptide, or glycan on the solid phase or monolith.

16. The method of claim 12, wherein the analyzing of the at least one former glycopeptide fragment, or the at least one peptide fragment, or at least one glycan is done by mass spectrometry.

17. The method of claim 12, further comprising washing the at least one conjugated protein or the at least one glycoprotein with a buffer before being cleaved.

18. The method of claim 12, wherein before releasing the at least one peptide, glycan, or former glycopeptide fragment, the solid phase or monolith is washed to remove the non-conjugated molecules.

19. The method of claim 12, wherein the at least one protein or the at least one glycoprotein is cleaved with a protease or a chemical to release at least one global peptide.

20. The method of claim 19, wherein the at least one protein or the at least one glycoprotein is cleaved with trypsin to release at least one global peptide.

21. The method of claim 12, wherein the at least one former glycopeptide fragment is released from the solid phase or monolith with a glycosidase or chemicals.

22. The method of claim 21, wherein the glycosidase is selected from the group consisting of an N-glycosidase for releasing a formerly N-glycopeptide and a β-elimination for releasing a formerly O-glycopeptide.

23. The method of claim 22, wherein the N-glycosidase is peptide-N-glycosidase F (PNGase F).

24. The method of claim 12, wherein the at least one glycan is released from the solid phase or monolith with a glycosidase or a chemical.

25. The method of claim 24, wherein the glycosidase is selected from the group consisting of an N-glycosidase for releasing N-glycan.

26. The method of claim 25, wherein the N-glycosidase is peptide-N-glycosidase F (PNGase F) for releasing N-glycan.

27. The method of claim 24, wherein the chemical is β-elimination for releasing O-glycan.

28. The method of claim 12, wherein the plurality of samples is selected from the group consisting of samples comprising a body fluid, a secreted protein, and a cell surface protein.

29. The method of claim 12, wherein the method further comprises the use of a liquid handling robot system.

30. The method of claim 12, wherein the chemical moiety comprises a hydrazide moiety.

31. The method of claim 30, wherein the hydrazide moiety comprises a hydrazide resin.
